(19) United States
(12) Patent Application Publication
(10) Pub. No.: US 2007/0265350 A1
(43) Pub. Date: Nov. 15, 2007

(54) METHOD OF IDENTIFYING COMPOUNDS USEFUL TO TREAT NEURONAL DEGENERATIVE DISEASES

(75) Inventors: John F. Engelhardt, Iowa City, IA (US); Weihong Zhou, Iowa City, IA (US); Maged M. Harraz, Iowa City, IA (US); Jennifer J. Marden, Iowa City, IA (US)

Correspondence Address: SCHWEGMAN, LUNDBERG & WOESSNER, P.A., P.O. BOX 2938, MINNEAPOLIS, MN, 55402, US

(73) Assignee: UNIVERSITY OF IOWA RESEARCH FOUNDATION, IOWA CITY, IA (US)

(21) Appl. No.: 11/617,491

(22) Filed: Dec. 28, 2006

Related U.S. Application Data
(60) Provisional application No. 60/755,337, filed on Dec. 30, 2005.

(51) Int. Cl. A61K 31/122; A61P 25/00; C07K 14/00; G01N 33/53; G01N 33/567

(52) U.S. Cl. 514/689; 435/7.2; 435/7.8; 530/300; 530/324; 530/350

(57) ABSTRACT

Methods of identifying agents that inhibit ROS by altering the binding of a GTPase such as Rac to SOD, agents identified by the method, and methods of using compounds that inhibit ROS to inhibit or treat neuronal degenerative diseases, are provided.

### CROSS REFERENCE TO RELATED APPLICATIONS

This application claims the benefit of the filing date of U.S. application Ser. No. 60/755,337, filed on Dec. 30, 2005, the disclosure of which is incorporated by reference herein.

### STATEMENT OF GOVERNMENT RIGHTS

The invention was made at least in part with grants from the Government of the United States of America (grant numbers DK067928 and DK51315 from the National Institute of Diabetes and Digestive Kidney Diseases). The Government may have certain rights in the invention.

### BACKGROUND

The regulation of reactive oxygen species (ROS) production by the GTPase Rac1 is important for many cellular processes involved in signal transduction (Sulciner et al., 1997), actin cytoskeletal rearrangements (Kheradmand et al., 1998), cell migration (Yamaoka-Tojo et al., 2004), proliferation (Irani et al., 1997, and differentiation (Puceat et al., 2003). ROS scavenging enzymes play an important role in maintaining cellular redox homeostasis by controlling levels of ROS, i.e., [symbol] O₂ and H₂O₂. Copper/zinc superoxide dismutase (SOD1) is a ubiquitously expressed cytosolic enzyme that regulates intracellular ROS through the conversion of [symbol] O₂→H₂O₂ (McCord et al., 1969). An important source of cellular ROS is NADPH-oxidases, for which seven known NADPH oxidase catalytic subunits exist (Nox1, Nox2^{gp91phox}, Nox3, Nox4, Nox5, Duox1, and Duox2) (Lambeth et al., 2004). NADPH oxidases generate superoxide ( [symbol] O₂) by transferring an electron from NADPH to molecular oxygen. The most widely characterized NADPH oxidase is phagocytic gp91phox (Nox2), which is also expressed in a variety of other nonphagocytic cell types. Rac1 is a central activator of Nox2, along with three other subunits of the Nox complex (p40phox, p47phox, and p67phox) (Lambeth et al., 2004). Despite the identification of numerous factors involved in ROS catabolism and metabolism, the mechanisms by which cells maintain redox-homeostasis remain poorly understood.

What is needed is a method to identify agents that alter levels of ROS.

### SUMMARY OF THE INVENTION

The invention provides a method to identify one or more agents that inhibit the production of ROS associated with regulation of a GTPase, e.g., Rac, by SOD. As described herein, SOD1 was found to activate Rac1 through a direct redox-regulated interaction that inhibits the intrinsic and GAP-stimulated GTP hydrolysis by Rac1, thereby actively regulating cellular [symbol] O₂ production via Nox2^{gp91phox}. Thus, SOD1-mediated activation of Rac1 which is controlled by ROS-sensitive binding, produces a self-regulating redox sensor for Nox2-dependent [symbol] O₂ production. A 35 amino acid segment of Rac1 was identified that specifically bound to SOD1, and that region is likely useful to identify agents that modulate the interaction between Rac1 and SOD1. In addition, SOD binds RhoA, another GTPase. Thus, SOD may regulate one or more NADPH oxidases, including but not limited to Nox1, Nox2, Nox3, Nox4, Nox5, Duox1, Duox2. Moreover, as SOD binds GDP (see FIG. 10) and guanine nucleotide binding motifs in SOD are conserved (see FIG. 11), agents that block (prevent or inhibit or otherwise alter) nucleotide binding, e.g., guanine nucleotide binding, to SOD may also be useful in the regulation of the production of ROS.

In one embodiment, the invention provides a method to identify one or more agents which regulate, e.g., prevent, inhibit or enhance, the binding of SOD to a GTPase, for instance, Rac. “SOD” as used herein, is a protein or polypeptide including SOD1 and SOD2 having at least 80%, 85%, 90%, 95% or more, e.g., 100%, amino acid sequence identity to SEQ ID NO:6 (human SOD1) or SEQ ID NO:7 (human SOD2), and optionally having superoxide dismutase activity. A SOD protein or polypeptide binds Rac, or another GTPase (see FIG. 12A) that regulates Nox or Duox, which interaction regulates NADPH oxidase. In one embodiment, the method includes contacting one or more agents, isolated or purified GTPase such as Rac protein which includes a SOD binding region, and SOD protein which includes a GTPase, e.g., a Rac or RhoA, binding region. In another embodiment, the method includes contacting one or more agents, GTPase such as Rac which includes a SOD binding region, and isolated or purified SOD protein which includes a GTPase binding region. In another embodiment, the method includes contacting one or more agents, isolated or purified GTPase, for instance, isolated or purified Rac, with a SOD binding region, and isolated or purified SOD protein which includes a GTPase binding region. Preferably, the binding region includes at least 10, e.g., 20, 25, 30, or 35, contiguous residues corresponding to residues in a wild-type GTPase or SOD, although smaller fragments are also envisioned. A control reaction may employ a constitutively active GTPase, e.g., a dominant negative Rac or alsin, which interacts with Rac and may activate Nox, e.g., in neurons.

In one embodiment, the invention provides an in vitro method to identify agents that specifically inhibit the interaction of Rac (or another SOD binding GTPase) and SOD. In another embodiment, the invention provides an in vitro method to identify agents that specifically enhance the interaction of Rac or another SOD binding GTPase and SOD. In one embodiment, the invention provides a method which includes contacting one or more agents, isolated Rac protein or another SOD binding GTPase, and SOD protein under conditions that allow for binding of the SOD binding GTPase to SOD. Then it is detected or determined whether the one or more agents inhibit or enhance binding of the isolated Rac protein or another SOD binding GTPase to the SOD protein. The detection or determination of binding, or the inhibition or enhancement thereof, can be accomplished by a variety of methods, some of which are described herein. For example, a GTPase such as Rac or a portion thereof which includes a SOD binding region, or SOD or a portion thereof which includes a GTPase binding region, may be labeled or may bind to a detectable label such as a labeled antibody, and/or may be fused to a heterologous peptide, e.g., fused to GST or a His tag, which facilitates isolation and optionally detection of the fusion protein. Alternatively, or in addition to, the one or more agents may be labeled or bind to a detectable label. Thus, assays such as fluorescence resonance energy transfer assays, luminescence resonance energy transfer assays, cleavage assays (protease or nuclease cleavage), crosslinking assays, scintillation proximity assays, fluorescence perturbation assays, nuclear magnetic resonance, and the like may be employed to detect or determine whether an agent inhibits or enhances binding of a GTPase, e.g., Rac or RhoA, to SOD. The methods may include whole cells, cell lysates or be cell-free, e.g., use isolated or purified GTPase and/or SOD. In particular, the method may be used to screen chemical libraries to identify agents which may be therapeutically useful or a candidate for rational design of a drug.

In another embodiment, the method includes providing a mixture comprising one or more agents and a sample comprising a GTPase that binds SOD, e.g., Rac and SOD. The mixture is subjected to conditions that allow for binding of the GTPase to SOD, and it is determined whether the one or more agents inhibit or enhance the binding of the GTPase to the SOD protein.

Also provided is one or more agents identified by the methods of the invention. Further provided is a method of using those agents, as described below.

Further provided is an isolated peptide which binds SOD, wherein the peptide has at least 90% identity to SEQ ID NO:2 but is not full-length Rac1 (SEQ ID NO:1), full-length Rac2 (SEQ ID NO:3), or full-length RhoA (SEQ ID NO:4 or SEQ ID NO:5), e.g., for Rac, an isolated Rac peptide is less than 177 amino acid residues in length, and for RhoA, an isolated RhoA peptide is less than 193 amino acid residues in length. In particular, peptides useful in the screening methods include a GTPase of at least 20, e.g., at least 30, 35, 40, 50, 60, 70, 80 or more, for instance 100, 120 or 150, amino acid residues. Also provided is an expression cassette encoding a GTPase such as Rac or a fusion thereof, or a GTPase peptide such as a Rac peptide or a fusion thereof, an expression cassette encoding SOD or a fusion thereof, or a SOD peptide or a fusion thereof, a vector or host cell which includes an expression cassette of the invention, and isolated or purified a GTPase or SOD proteins, including fusion proteins comprising a GTPase or SOD or a peptide thereof which is capable of binding SOD or a GTPase, respectively.

As also described herein, Nox2 activation is dysfunctional in certain SOD1 mutants known to cause amyotrophic lateral sclerosis (ALS). ALS SOD1 mutants demonstrated elevated levels of Nox2-derived superoxide production in isolated vesicles and in ALS transgenic mice. Hence, hyperactivation of Nox2 might contribute to the progression of motor neuron degeneration in ALS G93A-SOD1 transgenic mice. In addition, certain SOD1 mutants associated with ALS were found to direct more persistent Nox activation in vitro and in vivo due to enhanced redox-insensitive binding of SOD1 to Rac1. Moreover, a Nox2 deletion was found to delay motor neuron degeneration and prolong the life of ALS mice, e.g., the life span of SOD mutants was nearly doubled and the rate of functional decline from first symptoms was prolonged significantly on the Nox2 gene knockout background. Interesting, Nox2 heterozygous mice also had prolonged life and significantly delayed onset of paralysis, suggesting that small changes in Nox2 function may substantially delay disease. Nox1 knockout mice also had a significant enhancement in life expectancy (see FIG. 13), although less pronounced than Nox2 knockout mice. Rac1 has been shown to regulate Nox1 and so combined dysregulation of Nox2 and Nox1 by mutant SOD1 may contribute to the progression of ALS.

Apocynin inhibits recruitment of p47phox (a co-activator of the Nox complex) to the Nox complex. Given that Nox2 appeared to control disease progression in the presence of a ALS mutant SOD1, apocynin was tested for prolongation of life and delay of onset of disease in mice having those mutants. The lowest tested dose of apocynin was found to prolong life expectancy and delay disease onset (see FIGS. 14 and 17-18).

Thus, agents that modulate the molecular interaction between SOD, e.g., SOD1, and GTPases such as Rac1 (phagocytic Rac2 also has a similar interaction), or otherwise inhibit NADPH oxidases, e.g., agents that inhibit Nox such as apocynin, may be therapeutically useful in diseases that are associated with or caused by excessive ROS through Nox2, and also likely Nox1 (which is also regulated by Rac1) or other NADPH oxidases, including neuron degenerative diseases such as motor neuron degenerative diseases, and diseases associated with mutant SOD. Therefore, the invention includes these agents and methods which employ these agents in a therapeutic amount, e.g., an amount effective to delay progression of motor neuron loss and paralysis and/or promote motor neuron survival, in diseases such as ALS or other diseases that involve excess ROS production as a result of the dysregulation of Nox2 by SOD1/Rac or NADPH oxidases by SOD/GTPase, or diseases associated with mutant SOD, e.g., a mutant with altered, e.g., enhanced, binding to Rac or another GTPase or altered nucleotide binding. For instance, the agents are useful to prevent, inhibit or treat, diseases including but not limited to Alzheimer's, Parkinson's and Huntington's, inflammatory disorders such as arthritis, or other acquired or inherited diseases, e.g., brain ischemia (cerebral ischemia), stroke, dementia including prion demientias, Down's syndrome, multiple sclerosis, methylmalonic acidaemia, d-2 hydroxyglutaric aciduria, retinal degeneration, Pick's disease, Lewy bodies related disorders, Friederich's ataxia, and neuronal ceroid lipofuscinosis.

As an agent that inhibits NADPH oxidase, e.g., apocynin, prolongs life and delays onset of disease in mice, those agents are useful in breeding colonies of mice with neuronal degeneration, in particular, in chow formulated with or in water having those agents.

Moreover, as alsin also regulates Nox activation and modulation of superoxides, and may bind the same region of Rac as SOD, agents that alter, e.g., inhibit, binding of alsin to Rac may alter Nox activation.

Further provided is a method to inhibit or treat a neuronal degenerative disease in a mammal. The method includes administering to a mammal in need thereof a composition comprising an effective amount of an inhibitor of the activity of NADPH oxidase, e.g., a compound of formula (I).

Also provided is a method to enhance ROS in a mammal. The method includes administering to a mammal in need thereof, e.g., a mammal having cancer, a composition comprising an effective amount of agent that enhance the interaction GTPase and SOD, e.g., constitutively active Rac or SOD mutants as described above.

The invention thus provides agents for use in medical therapy, e.g., to inhibit or treat neuronal degenerative diseases characterized by excessive ROS and those that result from dysregulation of GTPase/SOD, e.g., Rac/SOD1 control of Nox2, in an effective amount, e.g., an amount effective to delay progression of motor neuron loss and paralysis or promote motor neuron survival in diseases that involve excess ROS production. Also provided is the use of such agents for the manufacture of a medicament to delay progression of motor neuron loss and paralysis or otherwise to inhibit or treat neuronal degenerative diseases characterized by excessive ROS or diseases associated with mutant SOD, e.g., a mutant with altered, e.g., enhanced, binding to Rac or altered nucleotide binding. Further provided are agents that enhance ROS or dysregulate Rac/SOD1 control of Nox2, in an effective amount, e.g., to inhibit or treat cancer.

### BRIEF DESCRIPTION OF THE FIGURES

FIGS. 1A-F. Rac1 binds to SOD1 in a redox dependent manner. A) Rac1 was immunoprecipitated (IP) from heart, kidney, liver, and/or brain tissue of sod1+/+ or sod1−/− mice followed by Western blotting (WB) for SOD1 and Rac1. B) In vitro IP of purified His-tagged Rac1 and Cdc42 in the presence of purified bovine SOD1 followed by WB for SOD1, Rac1, and Cdc42. The His-tagged GTPases were preloaded with the indicated nucleotide analogs prior to incubation with SOD1. C) In vitro IP of the indicated nucleotide-loaded GST-tagged Rac1 mutants (or free GST) in the presence of purified SOD1 followed by WB for SOD1 and GST. D) In vitro IP of purified His-tagged Rac1 in the presence of purified native, demetalated, or remetalated bovine SOD1 followed by WB for SOD1 and Rac1. The His-tagged Rac1 was preloaded with the indicated nucleotide analogs prior to incubation with SOD1. Additionally, untreated His-tagged Rac1 and 300 μM DTT pre-reduced His-Rac1 were used for in vitro pull down assays with each of the three forms of SOD1. E) His-Rac1 was pre-reduced (300 μM DTT), loaded with GTPγS, and then treated with indicated concentrations of hydrogen peroxide (H₂O₂) before performing pull-down assays with SOD1. F) The indicated concentrations of DTT were added to the 300 μM H₂O₂-treated His-Rac1 sample shown in (E), and pull-down assays were performed with SOD1.

FIGS. 2A-H. SOD1 regulates Rac1 activation through a redox-dependent physical interaction. A) Schematic of GST-Rac1 deletion mutants used to define the SOD1 binding domain. B) In vitro EP of various GST11 tagged Rac1 deletion mutants in the presence of purified bovine SOD1. The number at the top of each lane corresponds to the GST-Rac1 fusion construct number in Panel A. The top panel is a WB for SOD1 following IP of GST and the bottom panel is a Coomassie stained gel of the purified fusion peptides used for IP. C) The GST-tagged PAK binding domain (GST-PBD) was used in pull-down assays to quantify GTP-Rac1 in sod1+/− or sod1−/− mouse brain lysates. Western blots show GTP-Rac1 and GST-PBD following glutathione precipitation and total SOD1 and Rac1 levels in crude lysates. D) Quantification of GTP-Rac1 levels from 13 sod1+/− and 7 sod1−/− mouse brains demonstrated a significant difference (p<0.001, student's t-test). E, F) Rac1 GTPase assays were performed in the presence or absence of (E) bovine SOD1 or (F) E. coli SOD and/or GST-tagged p29-GAP. His-tagged Rac1 was preloaded with γP32-GTP, and aliquots of the reaction were analyzed at various time points by thin layer chromatography for GTP hydrolysis by assessing the % 32Pi released from Rac1. G) GTPase assay for Rac1 and Cdc42 in the presence or absence of SOD1 and/or p29-GAP. The rate of 32Pi release from γP32-GTP is plotted. H) Rac1 GTPase assay in the presence or absence of SOD1 and/or 100 μM xanthine/100 mU xanthine oxidase (X/XO). I) Pull-down assays of GTP-6 loaded His-Rac 1 in the presence of SOD1 with or without a 15 minute exposure to X/XO-derived ROS. Conditions used in this assay were identical to the GTPase assay shown in (H). Data in all panels are representative of at least three independent experiments.

FIGS. 3A-E. SOD1 activates [symbol] O₂ production by NADPH oxidase in the endosomal compartment. A) Endosomes were isolated from primary mouse dermal fibroblasts (PMDFs) using iodixanol density gradient fractionation and fractions were evaluated for NADPH-dependent superoxide production using a lucigenin-based luminescent assay (top panel) and by Western blot for Nox2^{gp91phox}, Rac1, SOD1, and EEA1 (bottom panels). B) Western blot for SOD1 in the indicated subcellular fractions from Nox2^{gp91phox} wild type and knock out (KO) PMDFs. C) Lucigenin assays were used to assess the rate of NADPH-dependent [symbol] O₂ production in Fraction #10 vesicles from Nox2 wild type and KO PMDFs in the presence or absence of SOD1 and/or DPI (a general Nox inhibitor) (n=6). D) Vesicular fractions from primary mouse embryonic fibroblasts (PMEFs) were assessed for rates of NADPH-dependent [symbol] O₂ generation in the presence or absence of Bovine SOD1 (Bov.SOD1) or E. coli SOD (Bac.SOD) (n=3). E) The ability of Bov.SOD1 and Bac.SOD to degrade X/XO-derived [symbol] O₂ in a lucigenin-based assay (n=3). F) PMA-induced [symbol] O₂ generation by PMNs isolated from sod1+/+, sod1+/−, or sod1−/− mice was assessed using a cytochrome c reduction assay. * Significant difference when compared to sod1+/+ mice (p<0.05, student's t-test).

FIGS. 4A-G. SOD1 mutants associated with ALS demonstrate enhanced, redox-insensitive, binding to Rac1 and enhanced ability to inhibit Rac1-GTP hydrolysis and activate endosomal NADPH-dependent [symbol] O₂ production. A) Coomassie stained SDS-PAGE of purified bacterially expressed SOD1 proteins. Bovine SOD1 was used as a reference control and normally migrates faster than human SOD1 (data not shown). The Cu/Zn content of each SOD1 protein is given below the gel. B) In vitro IP of purified pre-reduced His-tagged Rac1-GTP-γS in the presence of the indicated human SOD1 proteins (wt, L8Q, or G10V) at a 1:10 molar ratio (Rac1:SOD1). The Rac1/SOD1 complexes were then divided into two parts and half was treated with X/XO derived ROS for 15 minutes at room temperature prior to IP of the His-tag. X/XO conditions included 100 mUnits of xanthine oxidase enzyme with a final xanthine concentration of 100 μM. Following IP, Western blots for SOD1 and Rac1 were performed. Long and short exposures of the SOD1 blot are shown to demonstrate enhanced binding of each of the mutant forms of SOD1 to Rac1. C) Rac1 GTPase assays were performed using native bovine SOD1 or the indicated bacterially expressed and purified human SOD1 proteins (wt, L8Q, or G10V). The molar ratio of Rac1:SOD1 is indicated. His-tagged Rac1 was preloaded with γP32-GTP and the rate of 32Pi release from γP32-GTP is plotted in the presence of increasing concentrations of each type of SOD1. D) A time course of NADPH-dependent [symbol] O₂ generation by isolated PMEF endosomes was measured in the presence or absence of human WT-SOD1 or L8Q-SOD1. Results in Panels C and D are representative of three experiments. E) Rac1 was immunoprecipitated (IP) from brain tissue of G93A-SOD1 transgenic mice or control littermates followed by Western blotting (WB) for SOD1 and Rac1. F and G) NADPH-dependent [symbol] O₂ production by total endomembranes derived from (F) brain and (G) liver tissues at the indicated ages of G93A-SOD1 or control transgenic littermates (N=3 for each experimental point).

FIGS. 5A-C. A) SOD1 activity gel for native bovine SOD1 (lane 1), demetalated bovine SOD1 (lane 2), and remetalated bovine SOD1 (lane 3). Zn and Cu content of each form of bovine SOD1 is given above the gel in moles of metal per moles of protein. B) In vitro pull-down assays of His-Rac1 or His-Cdc42 pre-reduced with 300 μM DTT, pre-loaded with GTPγS, and then incubated with bovine SOD1 prior to Hisprecipitation and Western blotting for Rac1, SOD1, and Cdc42. C) Cartoon of the 3-dimentional structure of the Rac1 polypeptide backbone (Hirshberg et al., 1997). Left panel demonstrates the switch I region (blue), switch II region (red), G2 region (magenta) and the G3 region (green). Right panel demonstrates the minimal Rac1 peptide that strongly bound SOD1 (red) spanning the switch I, switch II, G2, and G3 regions.

FIGS. 6A-B. SOD1 does not affect GTP loading of Rac1 and must be enzymatically active to influence Rac1 GTPase activity. A) His-tagged Rac1 was loaded with 35S-GTPγS in the presence or absence of SOD1. The proteins were bound to nitrocellulose membrane and the excess unbound radionucleotide was removed by washing. The remaining (bound) 35S-GTPγS was quantified by liquid scintillation spectrometry. Results depict the mean +/−SEM for N=3 independent experiments. B) Rac1 GTPase assays were performed in the presence or absence of purified native, demetalated, or remetalated bovine SOD1. His-tagged Rac1 was preloaded with γP32-GTP and the rate of 32Pi release from γP32-GTP is plotted. Results are representative of two experiments.

FIG. 7. Redox-sensor model for SOD1-mediated regulation of Nox2 ROS production through Rac. Under reducing conditions SOD1 is bound to Rac-GTP and stabilizes Rac activation by inhibiting intrinsic and GAP-mediated GTP hydrolysis. Increased Rac-GTP levels lead to activation of Nox2 and the production of [symbol] O₂. [symbol] O₂ generated by the Nox2 complex is converted to H₂O₂ by SOD1 or through spontaneous dismutation. As the local concentration of H₂O₂ rises, oxidation of Rac leads to the dissociation of SOD1. With SOD1 no longer bound to Rac-GTP, hydrolysis to Rac-GDP occurs more quickly leading to inactivation of the Nox2 complex. SOD1 can then recycle to repeat the process as Rac/Nox2 is reactivated. Through this mechanism, we propose that SOD1 can sense the local concentration of ROS at sites of Rac/Nox2 complex activation and control the activity of the complex.

FIG. 8. Time to failure on rotorod for the various indicated genotypes. Death normally occurred within a week after failing the rotorod. Animals were considered clinically dead and euthanized when they could not right themselves within 20 seconds after being placed on their back.

FIG. 9. Motor neuron counts in spinal cord of aged matched siblings for the indicated genotypes. There are three animal in each group and animals were euthanized at the time of clinical death for the ALS+/Nox2+/+ group. This ranged from about 125-135 days and one mouse from each of the four genotypes was harvested on the same day.

FIG. 10. SOD1 binds GTP and GDP in vitro. A) S³⁵ radiolabeled GTPγS was incubated with SOD1 for 2 hours at room temperature with different concentrations of magnesium chloride (MgCl₂). The binding reaction was stopped by boiling in SDS-containing buffer for 5 minutes. Samples were run on SDS-PAGE, then transferred to a nitrocellulose membrane. Radiolabeled nucleotide bound to SOD1 is shown using autoradiography. B) Surface plasmon resonance (SPR) analysis of SOD1 guanine nucleotide binding (GTP and GDP shown). The SPR chip surface is coated with bovine SOD1 protein. Sample containing GTP or GDP (5 mM) flows over the surface and the nucleotide-protein binding kinetics is monitored.

FIG. 11. Conserved guanine nucleotide binding motifs in SOD1. Sequence of SOD1 from different organisms from Candida albicans to Homo sapiens is aligned. The conserved, potential sequence that binds guanine nucleotide is marked. The sequence LKxD on SOD1 deviates with only one amino acid from the consensus N/TKxD for the guanidine ring binding motif in guanine nucleotides. The sequence GDNxxGCT on SOD1 is also conserved and deviates with one amino acid from the phosphate binding loop consensus GxxxxGKT/S. Both motifs are exposed on the surface of SOD1 crystal structure and solute accessible.

FIGS. 12A-B. Comparison between Rac1, Rac2, RhoA and Cdc42 sequence and differential binding of Rac1 and RhoA to SOD1. A) Amino acid sequence alignment of the SOD1 binding region on Rac1 compared to Rac2, RhoA and Cdc42. Rac2 has more than 97% identical amino acid sequence compared to Rac1 in the SOD1 binding region. RhoA on the other hand has 77.7% identical sequence and Cdc42 has 75% identical sequence to Rac1 at that region. B) Rac1, RhoA or Cdc42 were immobilized on magnetic beads then loaded or not with guanine nucleotide (as labeled) and bound to SOD1. After washing unbound proteins, samples were separated on an SDS-PAGE and immunoblotted for SOD1. Rac1 bound SOD1 only in the GDPβS bound state while Cdc42 did not bind SOD1 regardless of the nucleotide loaded. On the other hand RhoA bound SOD1 only in the GTPγS bound state.

FIG. 12C. Amino acid sequences of human Rac1 (SEQ ID NO:1), human Rac2 (SEQ ID NO:3), human RhoA (SEQ ID NOs: 4 and 5), human SOD1 (SEQ ID NO:6), human SOD2 (SEQ ID NO:7), and human alsin (SEQ ID NO:8).

FIG. 13. Comparison of Nox1 and Nox2 gene knockout (KO) on survival of SOD1-G93A mice. A) ALS mice lacking Nox1 (N=6) survived longer (163 days) than their Nox1 containing littermates (127 days, N=8). **p<0.0039. B) Survival of ALS mice on the Nox2 KO background was even more pronounced than survival on the Nox1 KO background.

FIG. 14. Effects of apocynin (30 mg/Kg) on survival and disease progression in SOD1-G93A mice. A) Probability of survival in nontreated (125 days) compared with apocynin treated (185 days) mice (***p<0.0001). B) Gait analysis of untreated compared with apocynin treated mice. At 114 days of age, untreated mice were exhibiting an impaired gait while apocynin treated mice had a normal gait. C) Average age of disease onset as determined by first observation of hind limb weakness in untreated (117.5 days, n=20) compared with apocynin treated (156.5 days, n=6) mice. D) Survival Index is the time between disease onset and clinical death and is a marker of disease progression. Disease progression was slower in apocynin treated mice (32 days, n=6) compared with untreated mice (11 days, n=20).

FIG. 15. Expression of SOD1 mutants, but not wild type (WT) SOD1, leads to activation of cellular Nox activity. A) NADPH-dependent superoxide production in total endomembranes from brain, spinal cord, and liver of non-transgenic or transgenic mice overexpressing WT-SOD1 or G93A-SOD1 (N=3 animals in each group). B) Dihydroethidium (DHE) fluorescent detection of superoxide in lumbar spinal cord sections from 120 day old non-transgenic or transgenic mice overexpressing WT-SOD1 or G93A-SOD1. DAPI staining demarcates nuclei in lower panels. C, D) Measure of superoxide production (C) or trypan-blue exclusion as a measure of cell death (D) in SH-SY (neuronal) or MO59J (glial) cells infected with adenoviral vectors expressing LacZ, WT-SOD1, or the indicated mutant SOD1. E) Superoxide production and percentage of cell death in SH-SY or MO59J cells following infection with the indicated adenoviral vectors followed by treatment with or without apocynin (100 μM) for 72 hours. F) Rac-GTP activation as determined by association with GST-Pak1 using spinal cord cell lysates from non-transgenic (control) or G93A-SOD1 transgenic mice at 120 days of age. Controls include lysate from control mice incubated with GTPγS (+) or GDPβS (−) prior to performing Pak1 pull-down assays.

FIG. 16. Increase in NADPH-dependent superoxide production of ALS brain and spinal cord tissues of hemizygous G93A-SOD1 transgenic mice. Superoxide production was inhibited by DPI (10 μM), but not by rotenone (100 μM), suggesting Nox is responsible for the enhanced ROS production.

FIG. 17. Treatment with the NADPH oxidase inhibitor apocynin increases lifespan and slows disease progression in mice hemizygous for the G93A-SOD1 transgene. A) Survival curve for mice treated with different doses of apocynin in their water beginning at 14 days of age. Number of mice (N) for each treatment group is shown along with median survival times in days. B) Survival data of male and female mice for each given dose of apocynin. Mice treated for eye infections with antibiotics are marked as boxes. Those mice that were unsuccessfully treated and died from eye infections are denoted by an X within the box. Circles denote animals that never contracted eye infection. The number of mice in each group (N) is given above the mean survival in days for each dose of apocynin. C) Relationship between age of disease onset (as determined by a 5% weight loss during a one week period) and apocynin dosage. D) Survival Index was measured as the time from disease onset until the animal reached clinical death for each of the given doses of apocynin. E) NADPH-dependent superoxide production (Nox activity) was measured in total membranes of lumbar spinal cord from end-stage G93A-SOD1 transgenic mice (about 120 days of age) either untreated or treated with apocynin (300 mg/kg) for 5 days prior to analysis (N=5 in each group). F) Dihydroethidium fluorescence in lumbar spinal cord of the same mice shown in (E).

FIG. 18. Treatment of hemizygous G93A-SOD1 transgenic mice with apocynin (300 mg/kg) at different ages after birth. Survival times of mice given apocynin in their drinking water at 14, 60, and 80 days of age (N=6 for each group). All mice were derived from three transgene negative sibling females and one G93A-SOD1 hemizygous male. Two consecutive litters from each female were analyzed.

FIG. 19. Treatment of G93A-SOD1 transgenic mice with different concentrations of apocynin and the number of motor neurons in the lumbar spinal cord at day 120.

### DETAILED DESCRIPTION OF THE INVENTION

I. Reactive Oxygen Species and NADPH Oxidase

In general, vertebrates possess two fundamental mechanisms to respond to infection, the innate and the acquired immune system (Fearon et al., 1996). Innate, or natural immunity is the ability to respond immediately to an infectious challenge, regardless of previous exposure of the host to the invading agent. Elements of the innate system include phagocytic cells, namely polymorphonuclear leukocytes (PMNs) and mononuclear phagocytes (e.g., macrophages), and the complement cascade of circulating soluble preenzymic proteins. These elements constitute a relatively nonspecific ‘pattern recognition’ system which has functional analogues in the immune system of a wide variety of multicellular organisms, including plants (Enyedi et al., 1992) and insects (Hoffmann et al., 1999). As such, these evolutionary ancient elements represent a rapid and sensitive surveillance mechanism of host defense when the organism is challenged with an invading microorganism previously ‘unseen’ by the host's immune system. In contrast to the innate system, adaptive immunity is restricted to vertebrates and represents a precisely tuned system by which host cells define specifically the nature of the invading pathogen or tumor cell (Janeway et al., 1994). Such precision, however, requires time for antigens to be processed and specific lymphocytes and antibodies to be generated. Therefore, the adaptive system is slower to respond to new challenges than is the innate system which lacks specificity (Fearon et al., 1996).

Granulocytes arise from pluripotent stem cells located in the bone marrow, and include eosinophils, basophils, and neutrophils. PMNs are the most numerous leukocytes in the human peripheral circulation, and take their name from their typically multilobed nucleus. The daily production of mature PMNs in a healthy adult is in the order of 10¹¹ cells. During acute infection or other inflammatory stresses, PMNs are mobilized from the marrow reservoir, containing up to 10 times the normal daily neutrophil requirement (Nauseef et al., 2000). PMNs are motile, and very plastic cells which allows them to move to sites of inflammation where they serve as a first line of defense against infectious microorganisms. For this purpose, PMNs contain granules filled with proteolytic and other cytotoxic enzymes (Schettler et al., 1991; Borregaard et al., 1997). Besides releasing enzymes, PMNs are also able to phagocytose and to convert oxygen into highly reactive oxygen species (ROS). Following phagocytosis, ingested microorganisms may be killed inside the phagosome by a combined action of enzyme activity and ROS production.

Upon activation, PMNs start to consume a vast amount of oxygen which is converted into ROS, a process known as the respiratory or oxidative burst (Babior et al., 1976; Babior et al., 1978). This process is dependent on the activity of the enzyme NADPH oxidase. This oxidase can be activated by both receptor-mediated and receptor-independent processes. Typical receptor-dependent stimuli are complement components C5a, C3b and iC3b (Ogle et al., 1988), the bacterium-derived chemotactic tripeptide N-formyl-Met-Leu-Phe (FMLP) (Williams et al., 1977), the lectin concanavalin A (Weinbaum et al., 1980), and opsonized zymosan (OPZ) (Whitin et al., 1985). Receptor-independent stimuli include long-chain unsaturated fatty acids and phorbol 12-myristate 13-acetate (PMA) (Schnitzler et al., 1997). Upon activation, the oxidase accepts electrons from NADPH at the cytosolic side of the membrane and donates these to molecular oxygen at the other side of the membrane, either at the outside of the cells or in the phagosomes containing ingested microorganisms. In this way, a one-electron reduction of oxygen to superoxide anion (.O₂—) is catalyzed at the expense of NADPH as depicted in the following equation:
2O₂+NADPH→2.O₂—+NADP⁺+H⁺
Most of the oxygen consumed in this way will not be present as .O₂—, but can be accounted for as hydrogen peroxide which is formed from dismutation of the superoxide radical (Hampton, 1998; Roos et al., 1984):
.O₂+e-+H⁺→H₂O₂

However, hydrogen peroxide (H₂O₂) is bactericidal only at high concentrations (Hyslop et al., 1995) while exogenously generated superoxide does not kill bacteria directly (Babior et al., 1975; Rosen et al., 1979) because of its limited membrane permeability. Therefore, a variety of secondary oxidants have been proposed to account for the destructive capacity of PMNs.

Hydroxyl radicals (.OH), formed by the iron catalyzed Fenton reaction, are extremely reactive with most biological molecules although they have a limited range of action (Samuni et al., 1988).

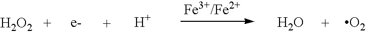

Singlet oxygen (102) is often seen as the electronically excited state of oxygen and may react with membrane lipids initiating peroxidation (Halliwell, 1978). Most of the H₂O₂ generated by PMNs is consumed by myeloperoxidase (MPO), an enzyme released by stimulated PMNs (Kettle et al., 1997; Nauseef, 1988; Zipfel et al., 1997; Klebanoff, 1999). This heme-containing peroxidase is a major constituent of azurophilic granules and is unique in using H₂O₂ to oxidize chloride ions to the strong non-radical oxidant hypochlorous acid (HOCl) (Harrison et al., 1976). Other substrates of MPO include iodide, bromide, thiocyanite, and nitrite (Van Dalen et al., 1997; Vliet et al., 1997).

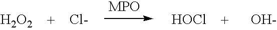

HOCl is the most bactericidal oxidant known to be produced by the PMN (Klebanoff, 1968), and many species of bacteria are killed readily by the MPO/H₂O₂/chloride system (Albrich et al., 1982).

In experimental settings, ROS production by activated phagocytes can be detected using enhancers such as luminol or lucigenin (Faulkner et al., 1993). For ROS-detection, lucigenin must first undergo reduction, while luminol must undergo one-electron oxidation to generate an unstable endoperoxide, the decomposition of which generates light by photon-emission (Halliwell et al., 1998). Luminol largely detects HOCl, which means that luminol detection is mainly dependent on the MPO/H₂O₂ system (McNally et al., 1996), while detection using lucigenin is MPO-independent and more specific for .O₂— (Anniansson et al., 1984). Luminol is able to enter the cell and thereby detects intra- as well as extracellularly produced ROS (Dahlgren et al., 1989), while lucigenin is practically incapable of passing the cell membrane and thereby only detects extracellular events (Dahlgren et al., 1985). However, results should be interpreted with care, because real specificity can never be assumed with any of these light-emission-enhancing compounds (Liochev et al., 1997).

Production of .O₂— seems to occur within all aerobic cells, to an extent dependent on O₂ concentration. In mitochondria, 1-3% of electrons are thought to form .O₂—. The fact that ROS are also quantitatively significant products of aerobic metabolism is illustrated by the following calculation: a normal adult (assuming 70 kg body weight) at rest utilizes 3.5 mL O₂/kg/min, which is identical to 352.8 l/day or 14.7 mol/day. If 1% makes .O₂— this gives 0.147 mol/day or 53.66 mol/year or about 1.7 kg of .O₂— per year. During the respiratory burst, the increase in O₂ uptake can be 10 to 20 times that of the resting O₂ consumption of neutrophils (Halliwell et al., 1998).

The NADPH oxidase, responsible for ROS production, is a multi-component enzyme system which is unassembled (and thereby inactive) in resting PMNs. However, activation of the phagocyte, e.g., by the binding of opsonized microorganisms to cell-surface receptors, leads to the assembly of an active enzyme complex on the plasma membrane (Clark, 1990; Segal et al., 1993). The critical importance of a functioning NADPH oxidase in normal host defense is most dramatically illustrated by the recurrent bacterial and fungal infections observed in individuals with chronic granulomatous disease (CGD), a disorder in which the oxidase is non-functional due to a deficiency in one of the constituting protein components (Smith et al., 1991; Dinauer et al., 1993; Segal et al., 1989; Dinauer et al., 1987; Volpp et al., 1988). PMNs from such patients, lacking a functionally competent oxidase, fail to generate .O₂— upon stimulation. Although the formation of ROS by stimulated PMNs may be a physiological response which is advantageous to the host, it can also be detrimental in many inflammatory states in which these radicals might give rise to excessive tissue damage (Weiss, 1989; Fantone et al., 1985; Jackson et al., 1988).

Essential components of the NADPH oxidase include plasma membrane and cytosolic proteins. The key plasma membrane component is a heterodimeric flavocytochrome b which is composed of a 91-kDa glycoprotein (gp91^{phox}) and a 22-kDa protein (p22^{phox}) (Rotrosen et al., 1992; Segel et al., 1992). Flavocytochrome b serves to transfer electrons from NADPH to molecular oxygen, resulting in the generation of .O₂—. In PMN membranes, a low-molecular-weight GTP-binding protein, Rap1A, is associated with flavocytochrome b and plays an important role in NADPH oxidase regulation in vivo (Quinn et al., 1989; Gabig et al., 1995). Furthermore, cytosolic proteins p47^{phox}, p67^{phox}, and a second low-molecular-weight GTP-binding protein, Rac2 are required for NADPH oxidase activity (Volpp et al., 1988; Lomax et al., 1989a; Lomax et al., 1989b) and these three proteins associate with flavocytochrome b to form the functional NADPH oxidase (Clark et al., 1990; Heyworth et al., 1991; Quinn et al., 1993; DeLeo et al., 1996). Additionally, a cytosolic protein, p40^{phox}, has been identified, but its role in oxidase function is not completely defined (Wientjes et al., 1993). According to the current model of NADPH oxidase assembly, p47^{phox} and p67^{phox} translocate en bloc to associate with flavocytochrome b during PMN activation (DeLeo et al., 1996; Park et al., 1992; Iyer et al., 1994). Rac2 translocates simultaneously, but independently of the other two cytosolic components, to associate with the membrane-bound flavocytochrome b (Heyworth et al., 1994; Dorseuil et al., 1995). Studies of oxidase assembly in PMNs of patients with various forms of CGD suggest that p47phox binds directly to flavocytochrome b (Heyworth et al., 1991) and at least six regions of flavocytochrome b have been identified as putative sites for interaction with p47^{phox}, including four sites on gp91phox and two sites on p22^{phox} (Kleinberg et al., 1990; Leusen et al., 1994; Leto et al, 1994; Leusen et al., 1994; Nakanish et al., 1992; DeLeo et al., 1995; Sumimoto et al., 1994; Finan et al., 1994).

II. Preparation of Reagents for Screening Assays and Screening Assays of the Invention

The present invention generally provides a method of screening for agents that specifically bind to an amino acid sequence in a region of a GTPase such as Rac corresponding to the region which binds SOD1. The method may employ isolated or purified peptides, polypeptides or fusion proteins which include the region, which peptides, polypeptides or fusion proteins are isolated from nonrecombinant cells (for peptides and polypeptides) or from in vitro transcription/translation systems, recombinant cells transfected with exogenous nucleic acid having an expression cassette encoding the peptide, polypeptide or fusion protein, or prepared by chemical synthesis. The method may also employ a cell which expresses the peptide, polypeptide or fusion protein from an expression cassette which is either transiently or stably introduced to the cell, yielding a recombinant cell. The expression cassette includes a promoter driving expression of the peptide, polypeptide or fusion protein. The promoter may be a constitutive promoter or a regulatable promoter, e.g., inducible.

Thus, the GTPase and SOD proteins employed in the screening methods may be recombinant or endogenous (native), and the assay may be a cell-free assay, e.g., one which employs isolated or purified Rac and isolated or purified SOD or employs a subcellular fraction to supply Rac and/or SOD, e.g., an endosomal fraction, or may be a cell-based assay, e.g., whole cells or cell lysates. In some assays that employ lysates or subcellular fractions, isolated, e.g., recombinant, GTPase or SOD may be added to a lysate or subcellular fraction which includes GTPase, SOD, or both GTPase and SOD.

In one embodiment, a test agent or a library of agents is contacted with Rac and that mixture contacted with SOD. In another embodiment, a test agent or library is contacted with SOD and that mixture contacted with GTPase. In one embodiment, a test agent or library of agent is contacted with GTPase and SOD, e.g., recombinant GTPase or SOD or a portion thereof which includes the appropriate binding region.

In one embodiment, the peptide, polypeptide or fusion protein having an amino acid sequence corresponding to the region of Rac1 that binds to SOD or corresponding to the region of SOD that binds Rac1, is coupled to a column, bead or other solid support, e.g., wells of a multi-well plate. In one embodiment, the peptide or polypeptide is one which is fused to other sequences, e.g., a glutathione S-transferase (GST) sequence, a His tag, calmodulin binding peptide, tobacco etch virus protease, protein A IgG binding domain, and the like, or a combination of sequences, useful to isolate, purify or detect the linked Rac or SOD polypeptide. In one embodiment, GST-Rac1 is immobilized on a support, e.g., a multi-well plate, and one or more agents and green fluorescent protein (GFP)-SOD are added simultaneously or sequentially to the immobilized Rac fusion protein. The amount or presence of GFP per well is detected or determined, and optionally compared to the amount or presence of GFP in a corresponding sample without agent addition.

Agents that modulate the binding of GTPase and SOD may modulate ROS production. Methods to detect the production of ROS and animal models of diseases associated with excessive ROS are known to the art. In particular, inhibitors of the binding of Rac and SOD are candidates for treating diseases characterized by excessive ROS, e.g., motor neuron disorders.

A. Definitions

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

The term “isolated” when used in relation to a nucleic acid, peptide, or polypeptide refers to a nucleic acid sequence, peptide or polypeptide that is identified and separated from at least one contaminant nucleic acid, polypeptide or other biological component with which it is ordinarily associated in its natural source. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “polypeptide” and protein” are used interchangeably herein unless otherwise distinguished, and “peptide” generally refers to a portion of a full-length polypeptide or protein or an amino acid sequence useful to isolate, purify or detect a linked sequence.

“Transfected,” “transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, 1972. The two sequences or parts thereof are more preferably homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80 percent sequence identity, preferably at least about 90 percent sequence identity, more preferably at least about 95 percent sequence identity, and most preferably at least about 99 percent sequence identity.

As used herein, “substantially pure” or “purified” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

B. Preparation of Expression Cassettes

To prepare expression cassettes encoding GTPase, for instance, Rac, SOD, a peptide thereof, or a fusion thereof, for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3N to 5N rather than 5N to 3N). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild-type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target of the lymphotrophic virus. Such promoters include the CMV promoter, as well as the SV40 late promoter and retroviral LTRs (long terminal repeat elements), although many other promoter elements well known to the art may be employed, e.g., the MMTV, RSV, MLV or HIV LTR in the practice of the invention.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, puro, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein.

The recombinant DNA can be readily introduced into the host cells, e.g., mammalian, bacterial, yeast or insect cells, or prokaryotic cells, by transfection with an expression vector comprising the recombinant DNA by any procedure useful for the introduction into a particular cell, e.g., physical or biological methods, to yield a transformed (transgenic) cell having the recombinant DNA so that the DNA sequence of interest is expressed by the host cell. In one embodiment, the recombinant DNA is stably integrated into the genome of the cell.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA viral vectors. Viral vectors, e.g., retroviral or lentiviral vectors, have become a widely used method for inserting genes into eukaryotic cells, such as mammalian, e.g., human cells. Other viral vectors can be derived from poxviruses, e.g., vaccinia viruses, herpes viruses, adenoviruses, adeno-associated viruses, baculoviruses, and the like.

To confirm the presence of the recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e.g., by immunological means (ELISAs and Western blots) or by other molecular assays.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

C. Peptides, Polypeptides and Fusion Proteins

The peptide, polypeptide or fusion proteins of the invention can be synthesized in vitro, e.g., by the solid phase peptide synthetic method or by recombinant DNA approaches (see above). The solid phase peptide synthetic method is an established and widely used method. These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, chemically modified derivatives of a given peptide, polypeptide, or fusion thereof, can be readily prepared. For example, amides of the peptide, polypeptide, or fusion thereof of the present invention may also be prepared by techniques well known in the art for converting a carboxylic acid group or precursor, to an amide. A preferred method for amide formation at the C-terminal carboxyl group is to cleave the peptide, polypeptide, or fusion thereof from a solid support with an appropriate amine, or to cleave in the presence of an alcohol, yielding an ester, followed by aminolysis with the desired amine.

Salts of carboxyl groups of a peptide, polypeptide, or fusion thereof may be prepared in the usual manner by contacting the peptide, polypeptide, or fusion thereof with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the peptide, polypeptide, or fusion thereof may be prepared by utilizing an N-acyl protected amino acid for the final condensation, or by acylating a protected or unprotected peptide, polypeptide, or fusion thereof. O-acyl derivatives may be prepared, for example, by acylation of a free hydroxy polypeptide or polypeptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the polypeptide. Other amino-terminal modifications include aminooxypentane modifications.

In one embodiment, a Rac or Rho peptide, polypeptide or fusion therewith has substantial identity, e.g., at least 80% or more, e.g., 85%, 90% or 95% and up to 100%, amino acid sequence identity to a wild-type Rac or Rho protein sequence corresponding to SEQ ID NO:1, SEQ ID NO:3, SEQ ID NO:4 or SEQ ID NO:5, for instance, substantial identity to residues from about residue 35 to about residue 70 of SEQ ID NO:1, and optionally binds SOD with an efficiency of at least 1%, 20%, 50% or more, e.g., 100%, 110% or more, relative to the efficiency of wild-type Rae or Rho binding to SOD. Thus, a peptide of Rac or Rho or a substituted Rac or Rho may bind wild-type SOD (or a mutant SOD) with a reduced, substantially the same, or an enhanced efficiency relative to a wild-type (full-length) Rac or Rho. “About” as used herein with respect to a particular residue means within 5 residues of the specified residue, e.g., within 1, 2, 3, 4 or 5 residues of residue “X” corresponding to residue “X” in a particular sequence. In one embodiment, a Rac peptide of the invention has SEQ ID NO:2 or an amino acid sequence with 80%, 85%, 90%, 95% or 99% identity to SEQ ID NO:2, e.g., a peptide having TVFD/ENYS/VAN/DV/IM/EVDG/SKP/QVN/ELG/ALWDTAGQEDYDRLRPL or an amino acid sequence with 80%, 85%, 90%, 95%, or 99% identity thereto, which binds SOD.

Substitutions of amino acids in Rac may include substitutions which utilize the D rather than L form, as well as other well known amino acid analogs, e.g., unnatural amino acids such as α,α-disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoyl-phenylalanine, phenylglycine, propargylglycine, sarcosine, ε-N,N,N-trimethyllysine, ε-N-acetyllysine, N-acetylserine, N-formylmethionine, 3-methylhistidine, 5-hydroxylysine, ω-N-methylarginine, and other similar amino acids and imino acids and tert-butylglycine.

Conservative amino acid substitutions are preferred—that is, for example, aspartic-glutamic as acidic amino acids; lysine/arginine/histidine as polar basic amino acids; leucine/isoleucine/methionine/valine/alanine/proline/glycine non-polar or hydrophobic amino acids; serine/threonine as polar or hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide, polypeptide or fusion polypeptide can readily be determined by assaying the specific activity of the peptide, polypeptide or fusion polypeptide.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic; trp, tyr, phe.

The invention also envisions a peptide, polypeptide or fusion polypeptide with non-conservative substitutions. Non-conservative substitutions entail exchanging a member of one of the classes described above for another.

Acid addition salts of the peptide, polypeptide or fusion polypeptide or of amino residues of the peptide, polypeptide or fusion polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

The peptides or polypeptides of the invention may be labeled, e.g., with a fluorophore or other detectable moiety, and/or fused to a peptide or polypeptide such as GFP, RFP, BFP and YFP, which may facilitate detection of Rac and SOD binding. Labels and peptides which may facilitate detection (or isolation and purification) include but are not limited to a nucleic acid molecule, i.e., DNA or RNA, e.g., an oligonucleotide, a protein, e.g., a luminescent protein, a peptide, for instance, an epitope recognized by a ligand, for instance, maltose and maltose binding protein, biotin and avidin or streptavidin and a His tag and a metal, such as cobalt, zinc, nickel or copper, a hapten, e.g., molecules useful to enhance immunogenicity such as keyhole limpet hemacyanin (KLH), cleavable labels, for instance, photocleavable biotin, a fluorophore, a chromophore, and the like.

III. Exemplary Compounds Useful in the Therapeutic Methods of the Invention

A. Definitions

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound disclosed herein may display superior activity compared with the other. When required, separation of the racemic material can be achieved by HPLC using a chiral column or by a resolution using a resolving agent such as camphonic chloride as in Tucker et al. (1994). A chiral compound of Formula I may also be directly synthesized using a chiral catalyst or a chiral ligand, e.g., Huffman et al. (1995).

“Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou and Talalay (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the methods of the present invention. Such organisms include, but are not limited to, mammals such as humans. In the context of the invention, the term “subject” generally refers to an individual who will receive or who has received treatment for treatment of the disease or disorder.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., non-peroxide oxy (—O—), thio (—S—), carbonyl (—C(═O)—), carboxy (—C(═O)—), imine (C═NH), sulfonyl (SO) or sulfoxide (SO₂).

Specific and preferred values listed below for radicals, substituents, and ranges, are for illustration only; they do not exclude other defined values or other values within defined ranges for the radicals and substituents

“Alkyl” refers to a C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (n-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The alkyl can optionally be substituted with one or more alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. The alkyl can optionally be interrupted with one or more non-peroxide oxy (—O—), thio (—S—), carbonyl (—C(═O)—), carboxy (—C(═O)O—), sulfonyl (SO) or sulfoxide (SO₂). Additionally, the alkyl can optionally be at least partially unsaturated, thereby providing an alkenyl.

“Alkenyl” refers to a C₂-C₁₈ hydrocarbon containing normal, secondary, tertiary or cyclic carbon atoms with at least one site of unsaturation, i.e., a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂ CH₂CH₂CH₂CH═CH₂).

The alkenyl can optionally be substituted with one or more alkyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The alkylene can optionally be substituted with one or more alkyl, alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylene can optionally be interrupted with one or more non-peroxide oxy (—O—), thio (—S—), carbonyl (—C(═O)—), carboxy (—C(═O)O—), sulfonyl (SO) or sulfoxide (SO₂). Moreover, the alkylene can optionally be at least partially unsaturated, thereby providing an alkenylene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

The alkenylene can optionally be substituted with one or more alkyl, alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, The alkenylene can optionally be interrupted with one or more non-peroxide oxy (—O—), thio (—S—), carbonyl (—C(═O)—), carboxy (—C(═O)O—), sulfonyl (SO) or sulfoxide (SO₂).

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkenyl, alkoxy, halo, haloalkyl, hydroxy, hydroxyalkyl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkoxy, halo, haloalkyl, hydroxy, hydroxyalkyl, aryl, heteroaryl, heterocycle, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkoxy, halo, haloalkyl, hydroxy, hydroxyalkyl, aryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkoxy, halo, haloalkyl, hydroxy, hydroxyalkyl, aryl, heteroaryl, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino[3,2,1-jk]carbazol-3-ium iodide.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “imino” refers to —C═NH.

The term “nitro” refers to —NO₂.

The term “trifluoromethyl” refers to —CF₃.

The term “trifluoromethoxy” refers to —OCF₃.

The term “cyano” refers to —CN.

The term “hydroxy” or “hydroxyl” refers to —OH.

The term “oxy” refers to —O—.

The term “thio” refers to —S—.

The term “thioxo” refers to (═S).

The term “keto” refers to (═O).

The term “carbohydrate” refers to an essential structural component of living cells and source of energy for animals; includes simple sugars with small molecules as well as macromolecular substances; are classified according to the number of monosaccharide groups they contain. The term refers to one of a group of compounds including the sugars, starches, and gums, which contain six (or some multiple of six) carbon atoms, united with a variable number of hydrogen and oxygen atoms, but with the two latter always in proportion as to form water; as dextrose, {C₆H₁₂O₆}. The term refers to a compound or molecule that is composed of carbon, oxygen and hydrogen in the ratio of 2H:1C:1O. Carbohydrates can be simple sugars such as sucrose and fructose or complex polysaccharide polymers such as chitin and starch.

The carbohydrate can optionally be substituted with one or more alkyl, alkenyl alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy.

The sugar can be a monosaccharide, disaccharide, oligosaccharide, or polysaccharide. The sugar can have a beta (β) or alpha (α) stereochemistry, can have an (R) or (S) relative configuration, can exist as the (+) or (−) isomer, and can exist in the D or L configuration. For example, the sugar can be β-D-glucose.

The term “saccharide” refers to any sugar or other carbohydrate, especially a simple sugar or carbohydrate. Saccharides are an essential structural component of living cells and source of energy for animals. The term includes simple sugars with small molecules as well as macromolecular substances. Saccharides are classified according to the number of monosaccharide groups they contain.

The term “polysaccharide” refers to a type of carbohydrate that contains sugar molecules that are linked together chemically, i.e., through a glycosidic linkage. The term refers to any of a class of carbohydrates whose are carbohydrates that are made up of chains of simple sugars. Polysaccharides are polymers composed of multiple units of monosaccharide (simple sugar).

The term “oligosaccharide” refers to compounds containing two to ten monosaccharide units.

Suitable exemplary sugars include, e.g., ribose, glucose, fructose, mannose, idose, gulose, galactose, altrose, allose, xylose, arabinose, threose, glyceraldehydes, and erythrose.

As used herein, “starch” refers to the complex polysaccharides present in plants, consisting of α-(1,4)-D-glucose repeating subunits and α-(1,6)-glucosidic linkages.

As used herein, “dextrin” refers to a polymer of glucose with intermediate chain length produced by partial degradation of starch by heat, acid, enzyme, or a combination thereof.

As used herein, “maltodextrin” or “glucose polymer” refers to non-sweet, nutritive saccharide polymer that consists of D-glucose units linked primarily by α,-1,4 bonds and that has a DE (dextrose equivalent) of less than 20. See, e.g., The United States Food and Drug Administration (21 C.F.R. paragraph 184.1444). Maltodextrins are partially hydrolyzed starch products. Starch hydrolysis products are commonly characterized by their degree of hydrolysis, expressed as dextrose equivalent (DE), which is the percentage of reducing sugar calculated as dextrose on dry-weight basis.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in an claim of the invention, the total number will be determined as set forth above.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic. Obviously, numerous modifications and variations of the present invention are possible in light of the above teachings. It is therefore to be understood that within the scope of the appended claims, the invention may be practiced otherwise than as specifically described herein.

B. Exemplary Nox Inhibitors

The present invention provides a method to inhibit ROS by employing one or more agents that directly inhibit Nox, e.g., by inhibiting a subunit thereof, or indirectly inhibit Nox by inhibiting the binding of Rac or another GTPase to SOD.

Compounds of formula (I) are suitable potent and selective inhibitors of NADPH oxidase: 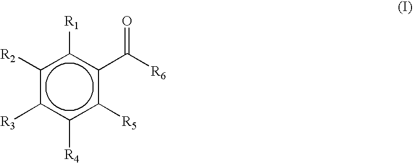
wherein,

R¹ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxy;

R² is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxy;

R³ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, O—R^{z}, NR^{x}R^{y} or COOR^{x}, wherein each R^{x} and R^{y} is independently H, alkyl, alkenyl, aryl, heteroaryl, heterocycle, cycloalkyl or hydroxy; and wherein R^{z} is a monovalent radical of a carbohydrate.

R⁴ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxy;

R⁵ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxy; and

R⁶ is H, alkyl, alkoxy, haloalkyl, hydroxy, hydroxyalkyl, aryl, heteroaryl, heterocycle, cycloalkyl, amino, alkylamino, acylamino, or NR^{x}R^{y}, wherein R^{x} and R^{y} are each independently H, alkyl, alkenyl, aryl, heteroaryl, heterocycle, cycloalkyl or hydroxy;

or a pharmaceutically acceptable salt thereof.

Compounds of formula (Ia) are suitable potent and selective inhibitor of NADPH oxidase: 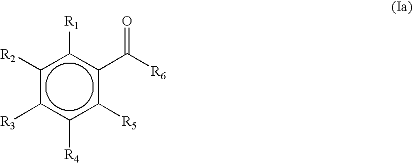
wherein,

R¹ is H;

R² is alkoxy;

R³ is hydroxyl, alkoxy or O—R^{z}, wherein R^{z} is a monovalent radical of a carbohydrate;

R⁴ is H, alkoxy or alkyl;

R⁵ is H or hydroxyl; and

R⁶ is alkyl, haloalkyl, hydroxyalkyl, aryl, heteroaryl, heterocycle, cycloalkyl, amino, alkylamino, or NR^{x}R^{y}, wherein R^{x} and R^{y} are each independently H, alkyl, alkenyl, aryl, heteroaryl, heterocycle, cycloalkyl or hydroxy;

or a pharmaceutically acceptable salt thereof.

Compounds of formula (Ib) are suitable potent and selective inhibitor of NADPH oxidase: 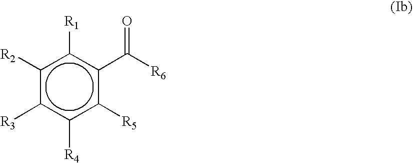
wherein,

R¹ is H;

R² is alkoxy;

R³ is hydroxyl, akloxy O—R^{z}, wherein R^{z} is a monovalent radical of a carbohydrate;

R⁴ is H, alkyl or alkoxy;

R⁵ is H or hydroxyl; and

R⁶ is alkyl;

or a pharmaceutically acceptable salt thereof.

Specific Ranges and Values:

Regarding the compound of formula (I): a specific value for R¹ is H; a specific value for R² is alkoxy; another specific value for R² is methoxy; a specific value for R³ is hydroxyl; another specific value for R³ is alkoxy substituted with hydroxyl; another specific value for R³ is 2-hydroxyl-ethoxy; another specific value for R³ is hydroxyl, a specific value for R⁴ is H; another specific value for R⁴ is alkoxy; another specific value for R⁴ is methoxy; another specific value for R⁴ is alkyl; another specific value for R⁴ is methyl; a specific value for R⁵ is H; another specific value for R⁵ is hydroxyl; a specific value for R⁶ is alkyl; and another specific value for R⁶ is methyl.

Regarding the compound of formula (Ia), a specific value for R² is alkoxy. Another specific value for R² is methoxy. A specific value for R⁶ is alkyl. Another specific value for R⁶ is methyl.

Regarding the compound of formula (Ib), a specific value for R² is alkoxy. Another specific value for R² is methoxy. A specific value for R⁶ is methyl.

A specific compound of formulas (I), (Ia) and (Ib) is apocynin. Apocynin (4-Hydroxy-3-methoxyacetophenone; acetovanillone; a compound of formula II), a cell-permeable phenol, is a potent and selective inhibitor of NADPH oxidase.

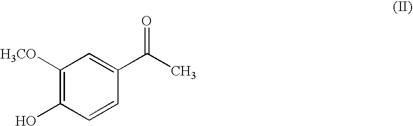

Apocynin is found in dry rhizomes and roots of Picrorhiza species, for example P. kurrooa and P. scrophulariiflora; the latter is also known as Neopicrorhiza scrophulariiflora. Apocynin may also be obtained from other sources, e.g., from the rhizome of Canadian hemp (Apocymum cannabinum) or other Apocynum species (e.g., A. androsaemifolium) or from the rhizomes of Iris species, provided that the extracts do not contain substantial amounts of cardiac glycosides. Picrorhiza kurroa Royle ex Benth is a perennial woody herb, and a crude extract there includes apocynin.

A Picrorhiza extract can be obtained by extracting the rhizomes of Picrorhiza species and subjecting the extract to column chromatography. Alternatively, extracts with high amounts of phenolic compounds can be obtained by pretreating the plant material with mineral acid to convert glycosides to their respective aglycones. If desired, the material may then be defatted to remove wax and other highly lipophilic matter. The material is extracted, for example with ethyl acetate and/or ethanol. The organic solvent is removed and an aqueous solution is obtained. The pH of the extract is increased to 10, e.g., with sodium hydroxide, to deprotonate phenolic compounds and to retain them in the aqueous phase. The aqueous solution is then washed, e.g., with diethyl ether to remove cucurbitacins. The aqueous phase is then reacidified to neutralise phenolic compounds and again extracted with, e.g., diethyl ether. The organic phase is collected and the solvent removed.

Additional suitable compounds of formula (I) include, e.g., compounds of the formula:

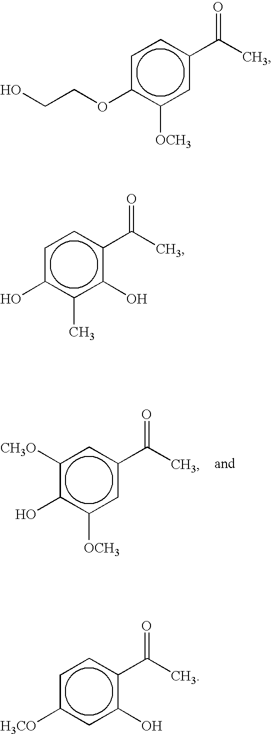

Other compounds useful in therapeutic or prophylactic methods to inhibit or prevent ROS include, but are not limited, to antioxidants in general, azelnidipine or other calcium antagonists, olmesartan or other AT1 receptor blockers, corticosteroids or glucocorticoids, e.g., dexamethazone or hydrocortisone, beta-adrenergic agonists, e.g., isoproterenol, lipocortin, pyridine, polyphenols, e.g., vanillin, 4-nitroguaiacol, folic acid and metabolic antagonists thereof, and imidazoles, as well as RNAi (see Example 2, or combinations thereof), and 4-(2-aminoethyl)benzenesulfonylfluoride.

In one embodiment, the agent is a statin, an ACE inhibitor, eicosanoid, phosphodiesterase inhibitor, phagocytophilium, antimicrobial peptide, e.g., PR-39, or one of those disclosed in U.S. Pat. Nos. 6,713,605, 6,184,203, 6,090,851, 5,990,137, 5,939,460, 5,902,831, 5,763,496, 5,726,551, and 5,244,916, U.S. published applications 20060154856, 20060135600, 20040043934, and 20040001818, and Cifuentes et al. (Curr. Op. Nephrol. & Hyperten., 15:179 (2006)), the disclosures of which are incorporated by reference herein.

C. Formulations and Dosages

The agents of the invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

The agents may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral administration, the active agent may be combined with one or more excipients and used in the form of ingestible tablets, buccal tablets, troches, capsules, elixirs, suspensions, syrups, wafers, and the like. Such compositions and preparations should contain at least 0.1% of active agent. The percentage of the compositions and preparations may, of course, be varied and may conveniently be between about 2 to about 60% of the weight of a given unit dosage form. The amount of active agent in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active agent may also be administered intravenously or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the agents may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the agents can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of the agent in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the agent, or an active salt or derivative thereof, required for use alone or with other agents will vary not only with the particular salt selected but also with the route of administration, the nature of the condition being treated and the age and condition of the patient and will be ultimately at the discretion of the attendant physician or clinician.

The agent may be conveniently administered in unit dosage form; for example, containing 5 to 1000 mg, conveniently 10 to 750 mg, most conveniently, 50 to 500 mg of active ingredient per unit dosage form.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. An apocynin containing composition may contain at least 50 μg, preferably at least 100 μg, up to 1000 mg of apocynin on the basis of daily intake. An example daily intake is between 1 and 100 mg apocynin; preferably a dosage of at least 15 mg/day. For instance, apocynin may be orally administered as a root powder in a dose of 375 mg three times in a day, by intramuscular injection of an alcoholic extract of the root of the plant daily (40 mg/kg) or by aerosol delivery administered in 8 doses for a total of 2 mg. An exemplary formulation and dosage include 300 to 500 mg root powder b.i.d./t.i.d. Moreover, analogs of apocynin may be used instead of or in addition to apocynin. Such analogs are in particular those in which the 4-hydroxyl group is etherified, especially with a hydroxylated alkyl group, such as 2-hydroxyethyl, 2,3-dihydroxypropyl or a sugar moiety. The latter analog in which the sugar moiety is β-D-glucose, is commonly known as androsin. This is the usual form in which apocynin is present in fresh plants.

The active ingredient may be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will be further described by the following non-limiting examples.

### EXAMPLE 1

### SOD1 is a Redox Sensor for Rac1-Mediated NADPH Oxidase Activation

Materials and Methods

Materials. Cytochrome C, phorbol myristate acetate (PMA), GTP, GDP, xanthine, xanthine oxidase, imidazole cellulose PEI matrix TLC plates, Lucigenin, β-NADPH and E. coli superoxide dismutase were purchased from Sigma-Aldrich corporation (St. Louis, Mo.). Dulbecco's modified Eagle's medium (DMEM), penicillin/streptomycin (P/S), 0.25% trypsin-EDTA, fetal bovine serum (FBS), Amphotericin B and collagenase were purchased from Invitrogen Corporation (Carlsbad, Calif.). Radioactive nucleotides, liquid scintillation fluid, Dextran 500 and nitrocellulose protein transfer membrane were purchased from Amersham Biosciences (Piscataway, N.J.). Protease inhibitor cocktail (PIC), EDTA-free PIC, GTPγS and GDPβS were purchased from Roche Applied Science (Indianapolis, Ind.). Histidine-tagged Rac1 (His-Rac1), His-Cdc42, Glutathione transferase-tagged (GST) p50-Rho-GAP catalytic domain (p29-GAP), GST-tagged wild type Rac1, V12Rac1 and N17Rac1 mutant fusion proteins were purchased from Cytoskeleton Inc. (Denver, Colo.). Bovine copper/zinc superoxide dismutase (SOD1) was purchased from Oxis Research (Portland, Oreg.). Dynabeads talon, dynabeads protein-A and protein-G were purchased from Dynal biotech (Lake Success, N.Y.). lodixanol, and Nycoprep 1.077 were purchased from Accurate Chemical & Scientific Corp. (Westbury, N.Y.).

Immunoprecipitation (IP) and Western blotting. SOD1 null mice (Sod1tm1Leb) were purchased from Jackson Laboratories (Matzuk et al., 1998). All animal experimentation was performed in accordance with the principles and procedures outlined in the NIH guidelines for the care and use of experimental animals. Tissue lysates from wild type and SOD1 knockout littermates were generated by homogenization in ice-cold PBS followed by the addition of an equal volume of 2× lysis buffer containing 40 mM Tris-HCl pH 7.4, 300 mM NaCl, 2% Triton X-100, 100 mM NaF, 80 mM β-glycerophosphate, 10 mM EDTA, and protease inhibitor cocktail tablet. Protein concentrations were measured by the Bradford assay. IP of Rac1 proteins was performed by incubating 600 μg of total protein with 4 μg of primary anti-Rac1 antibody (Upstate Cell Signaling Solutions Lake Placid, N.Y.) in 500 μl of lysis buffer. The IP reactions were rotated for 2 hours at 4° C. Protein A dynabeads (washed twice with lysis buffer) were added and rotated overnight at 4° C., followed by magnetic removal of the immunoprecipitated complexes. Beads were washed four times with lysis buffer. Pellets were then resuspended in SDS-PAGE reducing loading buffer and incubated at 98° C. for 5 minutes before separation by SDS-PAGE. Electrophoresis was performed using a Mini Protean II Bio-Rad unit with 0.75 mm gel slabs containing 10% (w/v) acrylamide in the separation gel and 4% acrylamide in the stacking gel, in 0.1% (w/v) SDS, 25 mM Tris-HCl-glycine buffer (pH 8.3). The nitrocellulose membranes bearing the transferred proteins were blocked overnight at 4° C. in blocking buffer containing 4% w/v non-fat dried milk and 0.3% Tween 20 in PBS, then incubated with primary antibodies to SOD1 (The Binding Site Limited Birmingham, UK) and Rac1 (Santa Cruz Biotechnology Inc. Santa Cruz, Calif.) and then with infrared dye-conjugated secondary antibodies. Protein bands were detected by the Odyssey infrared imaging system (LI-COR Biotechnology Lincoln, Nebr.).

Pull-down assays with GST- and His-tagged proteins. Dynabeads talon for histidine tagged proteins were washed with potassium phosphate buffer (PPHB) containing 100 mM KH₂PO₄, 10 mM NaCl, 0.25 mM MgCl₂ and 100 nM CaCl₂. 25 pmoles His-Rac1 or His-Cdc42 were incubated with the beads in PPHB at room temperature for 30 minutes with intermittent gentle agitation. The GTPases were either used directly or preloaded with either GTPγS or GDPβS and then washed. 250 pmoles of SOD1 was then added to each tube in PPHB. Samples were incubated at room temperature (RT) for 30 minutes with intermittent gentle agitation. Beads were then washed 3 times with PPHB to remove unbound SOD1. The fourth wash was carried out in 50 mM Tris pH 6.8. Proteins were eluted using 20 μl 125 mM imidazole and samples were then mixed with SDS-PAGE reducing loading buffer and separated by SDS-PAGE for Western blotting. For GST-Rac1, GST-V12Rac1, and GST-N17Rac1 pull down assays, a similar procedure was used with dynabeads protein G conjugated with anti-GST antibody (Santa Cruz Biotechnology Inc., Santa Cruz, Calif.).

Rac1-activation assays. Rac1 activation assays were performed using a previously described protocol with modifications described in Sanlioglu et al. (2001). Briefly, this assay utilizes a GST-PBD binding domain (Cytoskeleton) of PAK to specifically bind GTPRac1 (PBD encodes the p21 binding domain of Pak1). Brain tissue lysates were generated from wild type and SOD null littermate mice and normalized for protein concentration using the Bradford assay. GTP-bound Rac1 was precipitated from 2 mg of brain tissue lysate with GST-PBD using protein G dynabeads conjugated with anti-GST antibody. The immunoprecipitated pellet was evaluated by Western blotting for Rac1 and GST. The intensity of Rac1 immunoreactivity correlates with the level of GTP-bound Rac1 in the sample. Quantification of Western blots for GTP-Rac1 was performed on 13 heterozygous and 7 SOD null animals using infrared dye-conjugated secondary antibodies and an Odyssey infrared imaging system (LI-COR Biotechnology, Lincoln, Nebr.).

Guanine Nucleotide Exchange (GEF) Assay. GEF activity was assayed as previously described in Mansar et al. (1998) by measuring the incorporation of 35S-GTPγS into purified His-tagged Rac1. Briefly, 1 μCi of 35S-GTPγS was incubated with 250 pmol of His-tagged Rac1 in the presence or absence of 750 pmol of purified bovine SOD1 at 30° C. for 30 minutes with gentle agitation in GEF buffer containing 25 mM Tris-HCl, pH 8.0, 1 mM dithiothreitol, 5 mM EDTA, and 10 mM MgCl₂. The samples were filtered through nitrocellulose membrane and washed four times with washing buffer containing 25 mM Tris-HCl, pH 8.0, 100 mM NaCl, and 30 mM MgCl₂. Incorporated ³⁵S-GTPγS on Rac1 was measured using liquid scintillation spectrometry.

Rac1 GTPase assay. Rac1 GTPase assays were performed as previously described with modifications (Kwon et al., 2000). 25 μmol of His-Rac1 or His-Cdc42 were incubated with 25 pmol GTP and 2.5 pmol P32-labeled γ-GTP in GTP binding buffer containing 50 mM HEPES pH 7.6, 150 mM NaCl, and 0.1 mM EDTA for 10 minutes at room temperature and then placed in ice water. A 1 μl aliquot was then taken for thin layer chromatography (TLC) as time 0 of this GTPase reaction. Three proteins were added in various combinations to each reaction including bovine SOD1, E. coli SOD1, and/or p29-GAP. The ratio of Rac1 or Cdc42 to p29-GAP was 1:1. The ratio of Rac1 or Cdc42 to SOD1 was 1:10. To start the GTPase reaction, an equal volume of 2×GTPase buffer containing 50 mM HEPES pH 7.6, 150 mM NaCl, 10 mM EDTA, and 10 mM MgCl₂ was added to each condition at 15° C. Where indicated, 100 mU of xanthine oxidase were incubated in the reaction mixture with a final xanthine concentration of 100 μM. 1 μl aliquots were spotted on TLC plate from each sample at different time points. The TLC was run for 90 minutes at room temperature in 1 M acetic acid with 0.8 M LiCl running buffer. To quantify GTP hydrolysis, the free phosphate (Pi) bands were cut out along with the corresponding GTP bands. Each was put in liquid scintillation fluid and counted by liquid scintillation spectrometry. Percentage of GTP hydrolyzed was calculated by the equation, Pi/(Pi+GTP)×100.

Construction of GST-Rac1 and GST-SOD1 fusion proteins. Bacterial expression constructs for wild type, deletion mutants, and/or point mutants of GST-Rac1 and/or GST-SOD1 were generated by PCR-mediated cloning into the pGex-2T vector (Amersham Biosciences). All bacterial fusion constructs were confirmed by complete sequencing. ALS mutations L8Q (Bereznai et al., 1997) and G10V (Kim et al., 2003) were introduced into the GST-SOD1 using the Gene Editor in vitro site-directed mutagenesis system (Promega Madison, Wis.). The primer sequence used to generate the L8Q mutant was 5′-AAGGCCGTGTGCGTG CAGAAGGGCGACGGCCCA-3′ (SEQ ID NO:1). The primer sequence used to generate the G10V mutant was 5′-AAGGCCGTGTGCGTGCTGAAGGTTGACGGCCCA-3′ (SEQ ID NO:2).

Expression and purification of bacterial GST-tagged proteins. The GST-tagged expression constructs were transformed into E. coli using ampicillin selection. Bacterial colonies harboring the wild type and mutant constructs were grown in LB medium containing 100 μg/mL ampicillin in one liter flasks at 37° C. to a cell density of A₆₀₀=0.6. Isopropyl-D-thiogalactopyranoside (IPTG) was then added to 1 mM to induce the expression of GST-tagged proteins and cultures were grown for 6 hours at 37° C. The bacteria were collected by a 4000×g spin for 15 minutes at 4° C. and resuspended in PBS on ice. The bacteria were then lysed on ice by five 30-seconds sonicator pulses using a virsonic cell disruptor (VirTis Gardiner, N.Y.). The bacterial lysate was then centrifuged at 30,000×g for 30 minutes to pellet debris. The fusion proteins were purified from cellular extracts using glutathione-sepharose beads (Amersham Biosciences), according to the manufacturer's instructions, and the GST-fusion proteins were eluted with 10 mM glutathione, 50 mM Tris-HCl, pH 7.5, and 120 mM NaCl. The purity of fusion proteins was assessed by Coomassie stained SDS-PAGE and protein concentrations were normalized using the Bradford method. It should be noted that GST-Rac1 fusion proteins containing 88 or 116 amino acids of the N-terminus of Rac1 consistently migrated faster than their predicted molecular weights in SDS-PAGE and is likely due to altered folding properties of domains contained within these deletion mutants. The GST-tagged SOD1 proteins were cleaved from GST using a thrombin cleavage capture kit (EMD Bioscences San Diego, Calif.). Following cleavage, SOD1 proteins were separated from the cleaved GST-tag using an FPLC glutathione-sepharose column.

Demetalation of SOD1. Demetalation of purified bovine SOD1 was performed as previously described with modification (McCord et al., 1969). Copper and zinc were removed by exposing purified bovine SOD1 to pH 3.0 PBS, 2 mM EDTA, and stirring for 60 minutes at 4° C. The protein was then dialyzed overnight against 50 mM potassium phosphate pH 7.4. A fraction of demetalated bovine SOD1 was then remetalated by dialysis against 100 mM sodium acetate pH 5.5, in the presence of a 40-fold molar excess of Zn, followed by a 4-fold molar excess of Cu. To remove unbound metals, the SOD protein was then dialyzed several times against PBS pH 7.4. The Cu/Zn content of native, demetalated, and remetalated bovine SOD1 was determined as described in Ghezzo-Schoneich et al. (2001). Briefly, 10 μg SOD1 was mixed with 1 ml assay buffer containing 100 mM sodium borate, pH 7.8, 2% SDS, and 100 μM PAR. The reaction mixture was heated for 20 minutes at 100° C. Zn and Cu levels was calculated as the decrease in 500 nm reading measured on a Shimadzu UV-160 spectrophotometer after the addition of 0.8 mM NTA and EDTA, respectively. The Zn or Cu content in SOD1 is reported at the molar ratios of Zn or Cu to SOD1. SOD1 enzyme activity gels were performed as described in Zwacka et al. (1998). Briefly, 10 μg native, demetalated, or remetalated SOD1 was run on a native 12% polyacrylamide gel. SOD1 activity was determined using nitroblue tetrazolium reduction. Enzymatic activity is defined as the clearance zones in a background of black precipitate.

Subcellular Fractionation. Buoyant density centrifugation was used for subcellular fractionation and isolation of endosomes containing Nox2 activity. Cells were washed twice with ice-cold PBS and scraped into a 1.5 ml microfuge tube using the same buffer. The cells were pelleted and resuspended in homogenization buffer (HMB) containing 0.25 M sucrose, 20 mM HEPES pH 7.4, 1 mM EDTA, and an EDTA-free protease inhibitor cocktail. The cells were homogenized using nitrogen cavitation in a cell disruption high-pressure chamber (Parr instruments, Moline, Ill.). The pressure was raised to 650-psi for 5 minutes and released suddenly. The homogenate was centrifuged at 3000×g for 15 minutes to pellet unbroken cells, nuclei, and heavy mitochondria. The heavy mitochondrial supernatant (HMS) was bottom loaded into an iodixanol discontinuous gradient in a 12.5 ml SW41Ti ultracentrifuge tube using a previously described method with modifications (Xia et al., 1998; Graham et al., 1994). The discontinuous gradient was composed of 1.25 ml HMB without EDTA followed by bottom loading of the following % iodixanol steps sequentially with 1.0 ml 2.5%, 1.0 ml 5%, 1.5 ml 9%, 1.5 ml 14%, 2.5 ml 19%, 1.5 ml 26%, and finally the HMS in 2 ml 32%. Iodixanol concentrations were prepared fresh using a 50% iodixanol working solution (WS) diluted with HMB without EDTA. The WS was prepared by adding 1 part buffer containing 0.25 M sucrose, and 120 mM HEPES pH 7.4 to 5 parts iodixanol 60% stock solution. The gradients were centrifuged at 100,000×g using an SW41Ti swinging rotor overnight at 4° C. The fractions were collected from the top of the tube using a fraction collector (Labconco, Kansas City, Mo.) in 500 μl fractions on ice. The density gradient was designed to optimally separate the following compartments based on previous studies (Graham et al., 1994; Billington et al., 1998; Graham et al., 1996; Graham et al., 2002, Plorine et al., 1999): Fraction# 1-5 plasma membrane (density 1.03-1.05 g/ml); Fraction# 7-13 endosomal compartment (density 1.055-1.11 g/ml); Fraction# 8-10 Golgi apparatus (density 1.06-1.09 g/ml); Fraction# 10-13 light endoplasmic reticulum (density 1.09-1.11 g/ml); Fraction# 13-18 lysozomes (density 1.11-1.13 g/ml): Fraction# 18-21 light mitochondria (density 1.13-1.15 g/ml); Fraction# 19-20 heavy endoplasmic reticulum (density 1.145 g/ml); Fraction# 21-24 peroxisomes (density 1.18-1.2 g/ml); and Fraction# 22-24 cytosolic proteins (density 1.26 g/ml).

Lucigenin chemiluminescence (LCL) assay for NADPH-dependent superoxide (.O₂) production. NADPH oxidase activities were analyzed by measuring the rate of [symbol] O₂ generation using a chemiluminescent, lucigenin-based system (Li et al., 1998). 5 μM lucigenin in 50 μl of each subcellular fractions was incubated in the dark at room temperature for 15 minutes. LCL was measured using a single-tube Luminometer TD20-20 (Turner Designs Sunnyvale, Calif.). The reaction was initiated by the addition $-NADPH to a final concentration of 100 μM with or without DPI and/or SOD as indicated. LCL was measured over the course of 5 minutes. The initial slope of the luminescence curve (RLU/minute) was used to calculate the rate of luminescence product formation and compared between samples as an index of NADPH oxidase activity. In the absence of NADPH, the luminescence was negligible and did not change over time.

Primary mouse dermal fibroblast (PMDF) isolation. PMDFs were isolated from gp91phox(Nox2) KO heterozygous breedings pairs (Pollock et al., 1995). 1-day-old pups were euthanized, cleaned with sterile PBS, and their skins were removed immediately. Skin from each pup was separately placed with the dermal side down into a sterile 35 mm Petri dish and floated on 0.25% trypsin-EDTA overnight in 4° C. The following day, the epidermis was peeled off the dermis. The dermis was then incubated in 0.2% collagenase in DMEM for 1 hour at 37° C. The dermis was shaken to release the fibroblasts, this mixed cell population was pelleted and plated in DMEM with 10% FBS, 1% P/S, 2.5 units/ml amphotericin B, and 2 mM L-glutamine. Calcium was raised to 6 mM to induce calcium-dependent differentiation and detachment of contaminating keratinocytes. Following expansion of PMDFs, genomic DNA was generated from a subset of cells from each isolate for Nox2 genotyping.

Primary mouse embryonic fibroblast (PMEF) isolation. PMEFs were isolated from SOD1 KO heterozygous breedings pairs (Matzuk et al., 1998). Embryos were harvested from 14-day post coitus pregnant female mice. Following removal of the head and internal organs, embryos were rinsed in PBS, minced and incubated in 0.25% trypsin-EDTA overnight in 4° C. Trypsin was inactivated by adding DMEM with 10% FBS, 1% P/S, 2 mM L-glutamine, and 55 μM β-mercaptoethanol. The cells were washed and plated in the same media. Following expansion of PMEFs, genomic DNA was generated from a subset of cells from each isolate for SOD1 genotyping.

Isolation of polymorphonuclear leukocytes (PMNs) from mouse blood. PMNs were isolated as described in Freeman et al. (1991). Briefly, 1 ml of blood was collected from mice by cardiac puncture in a syringe preloaded with 1 ml of blood dilution buffer containing 0.85% (w/v) NaCl, 1 mM EDTA, 10 mM Hepes-NaOH pH 7.4. Erythrocytes were sedimented using dextran aggregation by incubating the diluted blood with 0.75 volume of 20% (w/v) polysucrose (dextran 500), in 0.85% (w/v) NaCl, 10 mM Hepes-NaOH, pH 7.4 for 30 minutes at room temperature. The leukocyte rich supernatant was then removed and layered upon 0.5 volume of Nycoprep 1.077 and centrifuged at 600×g for 20 minutes. The supernatant was discarded and PMNs resuspended in blood dilution buffer and used immediately.

Respiratory burst assay for .O₂ generation by mouse polymorphonuclear leukocytes (PMNs). [symbol] O₂ generation by intact PMNs was measured as described previously with modifications (Clark et al., 1987). Briefly, PMNs were adjusted to 10⁶ cells/ml. The cells were treated with 500 nM phorbol myristate acetate (PMA) or with vehicle (0.005% DMSO final concentration in blood dilution buffer) for 1 hour at 37° C. in the presence of 125 μM ferricytochrome c. [symbol] O₂ generation was measured in real time over a 1 hour period as SOD-inhibitable reduction of ferricytochrome c. The assays were conducted in 96 well plates with two wells for each experimental sample (one well with 30 μg bacterial SOD and one well without SOD). Reference wells were used to calculate the rate of SOD-inhibitable reduction of ferricytochrome c. Reduction of ferricytochrome c was detected by an absorbance change at 550 nm. The linear portion of the curve was used to calculate the reaction rate by linear regression analysis with R-square values over 0.90 for all samples.

Results

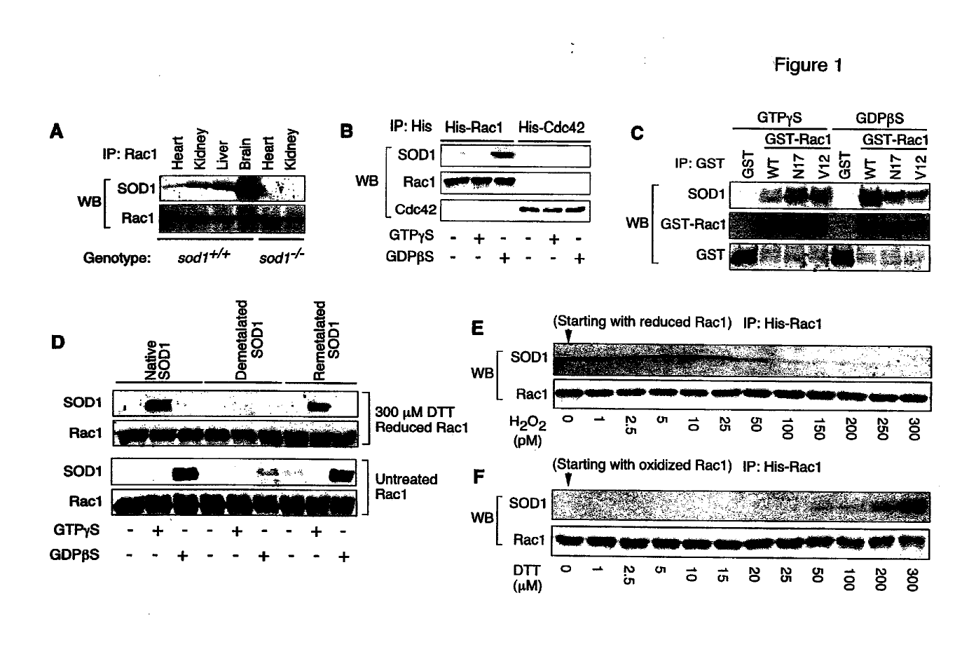

In an attempt to identify Rac1 binding partners important for regulating cellular ROS by NADPH oxidases, ectopically expressed HA-tagged Rac1 from mouse liver was immunoprecipitated and MALDI-TOF analysis performed on distinct bands seen in a SDS-PAGE. Surprisingly, SOD1 was identified as a potential binding partner to Rac1. To confirm that Rac1/SOD1 interactions occurred in vivo, co-immunoprecipitation experiments from several mouse organs including brain, liver, kidney, and heart were conducted. Indeed, immunoprecipitation of Rac1 pulled down SOD1 from each of these organs of sod1+/+, but not from sod1−/−, mice (FIG. 1A). The amount of SOD1 associated with Rac1 was noticeably highest in the brain and lowest in the heart. To test whether this interaction was direct, in vitro pull-down assays with purified proteins were utilized. Immobilized His-tagged Rac1 clearly associated with SOD1 when Rac1 was preloaded with GDPβS, but not when Rac1 was preloaded with GTPγS or in the absence of nucleotide (FIG. 1B). In contrast, the related Rho GTPase, Cdc42, did not associate with SOD1 (FIG. 1B). These results suggested that the GDP-bound form of Rac1 associates with SOD1.

To further investigate how potential nucleotide bound conformational states of Rac1 influenced association with SOD1, two Rac1 mutants which lock Rac1 in GTP (Rac1G12V) or GDP (Rac1 T17N) bound conformations were evaluated. However, GST-Rac1G12V or GST-Rac1T17N only weakly associated with SOD1, and the binding of SOD1 to either mutant was unaffected by the type of nucleotide loaded into Rac1 (FIG. 1C). In contrast, as previously shown with His-tagged wt-Rac1, GSTwt-Rac1 strongly associated with SOD1 when Rac1 was loaded with GDPβS, but not GTPγS.

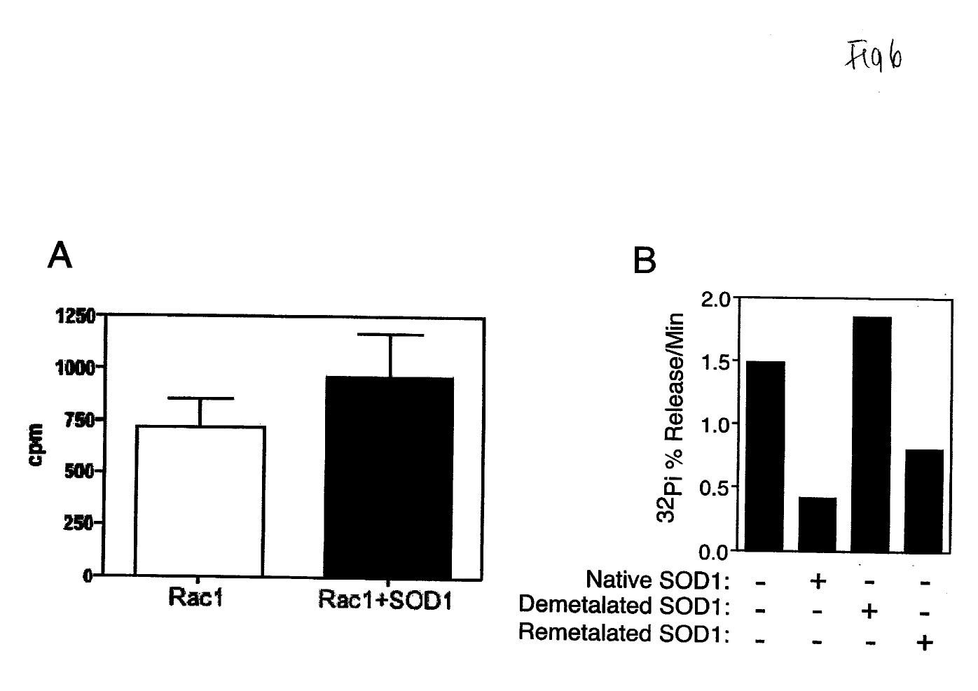

Given that Rac1 regulates [symbol] O₂ production through NADPH oxidases (Irani et al., 1997; Abo et al., 1991) and SOD1 dismutates [symbol] O₂→H₂O₂, it was hypothesized that SOD1 enzymatic activity might be fundamentally important for interactions with Rac1. Copper (Cu) binding at the active site of SOD1 is necessary for its enzymatic activity, and a specific Cu chaperone (CCS) is required for the loading of SOD1 with copper in vivo (Rac et al., 1999). Using in vitro pull-down assays, the redox regulation of the interaction between Rac1 and SOD1, and the effect the metal content of SOD1 on this interaction, was investigated. Interestingly, reduction of Rac1 switched the nucleotide preference required for binding to SOD1 (FIG. 1D). Non-reduced bacterially expressed Rac1 most efficiently bound to SOD1 in the presence of GDPβS. In contrast, reduced Rac1 bound to SOD1 when loaded with GTPγS but not GDPβS. Furthermore, only native (metalated) and remetalated forms of SOD1 bound to Rac1, while demetalated (enzymatically inactive) SOD1 failed to bind Rac1 (FIG. 1D and FIG. 5A). In contrast, neither reduced Cdc42-GTPγS or Cdc42-GDPβS bound SOD1 (FIG. 5B). These findings demonstrated that SOD1 can indeed bind Rac1-GTP under reducing conditions, and suggested that the redox-state of Rac1 influences its affinity for SOD1 in GTP vs GDP bound states.

Intrigued by these results, it was determined whether sequential reduction and oxidation of GTP-bound Rac1 could cycle Rac1 into SOD1 bound and unbound states, respectively. To this end, Rac1 (reduced with DTT and preloaded with GTPγS) was exposed to different concentrations of H₂O₂ and evaluated its ability to associate with SOD1 after removing excess H₂O₂. Results from these studies demonstrated that H₂O₂ concentrations as low as 50 μM caused a significant decrease in the binding affinity of Rac1 for SOD1 (FIG. 1E). To exclude the possibility of H₂O₂-mediated irreversible damage to Rac1 protein, the same experiment was repeated adding back different concentrations of DTT to oxidized Rac1 exposed to 300 pM H₂O₂. Indeed, H₂O₂-mediated inhibition of Rac1/SOD1 binding was reversed by treatment of Rac1 with 50-300 μM DTT (FIG. 1F). These in vitro association data demonstrated that Rac 1/SOD 1 binding is redox-regulated and can cycle between bound and unbound states depending on the redox state of Rac1.

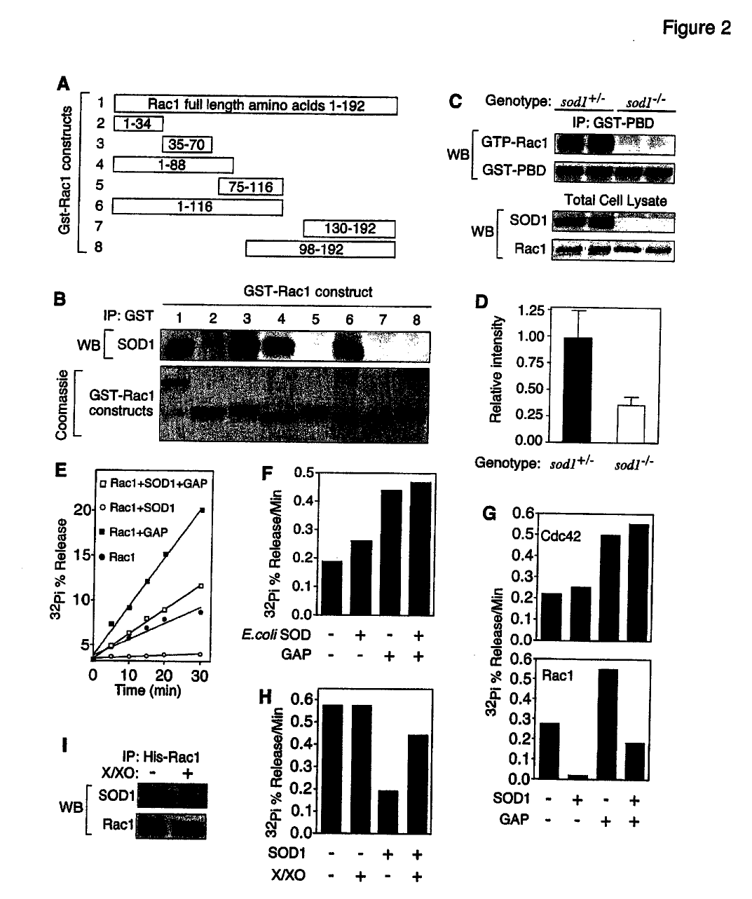

To determine the domain of Rac1 that associated with SOD1, GST-tagged deletion mutants of Rac1 (FIG. 2A) were constructed and in vitro pull-down assays conducted. SOD1 most efficiently bound a region of Rac1 contained within amino acids 35 to 70 (FIG. 2B). This region of Rac1 spans several domains important for nucleotide binding (i.e., switch I, G2, switch II, and G3 domains) (Hirshberg et al., 1997; Ito et al., 1997; Sprang et al., 1997) (FIG. 5C). Binding of SOD1 to this region on Rac1 is also consistent with the observed differences in binding between SOD1 and GTPγS- versus GDPβS-bound Rac1 and the reduced ability of Rac1T17N and Rac1G12V mutants (which both have mutations in the nucleotide-binding domain of Rac1) to associate with SOD1 (FIG. 1C).

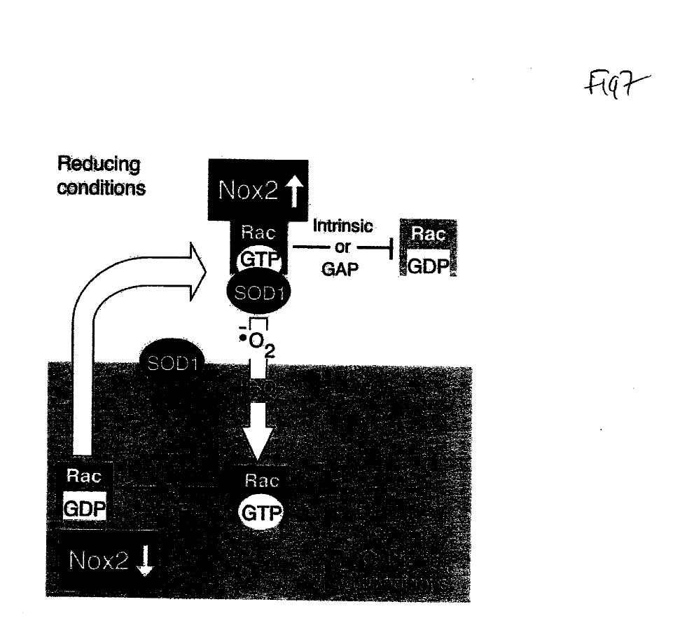

Interestingly, Rac1 guanine-nucleotide exchange factor (GEF) Tiam1 binds to a region of Rac1 that spans the interacting domain with SOD1 (Worthylake et al., 2000). In addition, the switch regions on two related Rho GTPases (RhoA and Cdc42) are involved in binding to RhoGAP (Rittinger et al., 1997a; Rittinger et al., 1997b). Therefore, it was hypothesized that SOD1 might influence Rac1 activity by acting as a GEF or GAP. To test this hypothesis, it was first determined whether cellular GTP-Rac1 levels were altered in the absence of SOD1. To this end, GST-PDB (the PAK domain which binds to GTP-Rac1) pull down assays were performed to assess the extent of GTP-Rac1 in sod1+/− and sod1−/− mice. Since brain tissue showed the most binding between SOD1 and Rac1 in vivo (FIG. 1A), these Rac1 activation assays were conducted in brain tissue lysates. Results from these experiments demonstrated that the level of GTP-bound (active) Rac1 was significantly higher in sod1+/−, as compared to sod1−/−, mouse brain tissue (FIGS. 2C and D). However, the total level of Rac1 in the brain was unaffected by the presence or absence of SOD1 (FIG. 2C). These findings demonstrated that SOD1 expression influences Rac1 activation in vivo by enhancing levels of GTP-bound Rac1. Unexpectedly, SOD1 did not significantly affect GTP loading on Rac1 in vitro (FIG. 6A). Therefore, SOD1 did not appear to function as a traditional GEF to increase levels of GTP-bound Rac1.

Since Rac1 has an exceptionally high intrinsic GTPase activity (Menard et al., 1992), it was determined whether SOD1 inhibited GTP hydrolysis by Rac1. As shown in FIG. 2E, this was indeed the case. SOD1 significantly inhibited the intrinsic GTPase activity of Rac1 and also prevented p29Rho-GAP from activating GTP hydrolysis by Rac1. However, inhibition of Rac1 GTPase activity was not seen with bacterial SOD (FIG. 2F), which did not associate with Rac1 in vitro (data not shown). The ability of SOD1 to inhibit GTP hydrolysis was also specific for Rac1 and was not observed with the closely related small GTPase Cdc42 (FIG. 2G). Furthermore, demetalated (enzymatically inactive) SOD1, which does not associate with Rac1 (FIG. 1D), also did not inhibit Rac1 GTPase activity (FIG. 6B). These findings demonstrated that SOD1 acts to specifically stabilize Rac1-GTP by inhibiting its GTPase activity.

Given that the binding of Rac1 to SOD1 was controlled by the redox-state of Rac1, SOD1 regulation of Rac1 GTPase activity might also be redox-regulated. To directly evaluate whether ROS alter the ability of SOD1 to inhibit GTP hydrolysis by Rac1, GTPase assays were performed in the presence of a xanthine/xanthine oxidase (X/XO) [symbol] O₂ generating system. Given that Rac1 regulates [symbol] O₂ production by certain NADPH oxidases, such a question was potentially relevant to processes that regulate ROS production in vivo. Interestingly, SOD1 lost its ability to inhibit GTP hydrolysis by Rac1 in the presence of this ROS generating system (FIG. 2H). However, the levels of ROS generated under the experimental conditions did not affect the intrinsic Rac1 GTPase activity in the absence of SOD1 (FIG. 2H). Immunoprecipitation of Rac1-GTPγS/SOD1 complexes using the GTPase assay conditions demonstrated that exposure to X/XO derived ROS dissociates SOD1 from Rac1 (FIG. 2I). These findings demonstrated that ROS alter the ability of SOD1 to regulate Rac1 GTPase activity by controlling physical interactions between these two proteins. These findings are consistent with the ability of H₂O₂ to disrupt SOD1/Rac1 interactions (FIG. 1E).

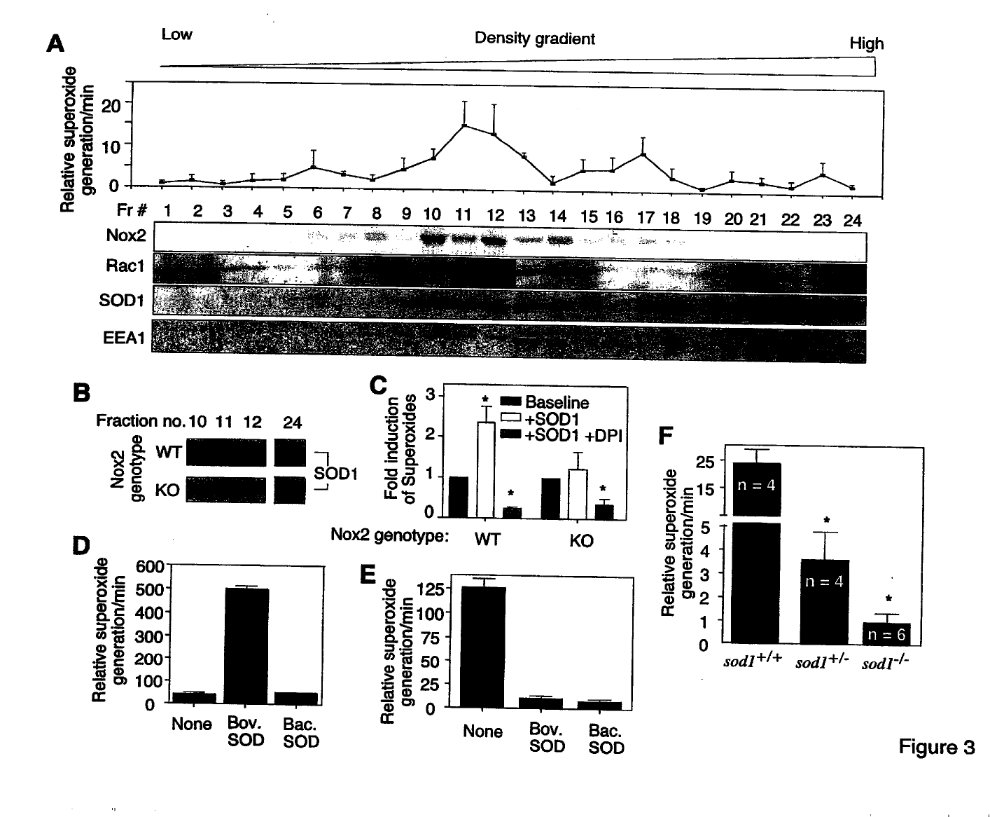

Rac1 is well recognized for its ability to regulate cellular [symbol] O₂ through its interactions with the NADPH oxidase Nox2^{gp91phox} (Lambeth et al., 2004). This interaction has placed Rac1 central to a number of ROS-regulated cellular processes controlled by [symbol] O₂ and/or H₂O₂ (the dismutated product of [symbol] O₂) (Sulciner et al., 1996; Kheradmand et al., 1998; Yamaoka-Tojo et al., 2004; Irani et al., 1997; Puceat et al., 2003). Interestingly, SOD1 is recruited to the surface of endosomes that produce Nox2-dependent [symbol] O₂ following IL-1β activation (Example 2). This led to the hypothesis that SOD1 might activate Rac1/Nox2 complexes in the endosomal compartment to produce [symbol] O₂ by inhibiting the GTPase activity of Rac1. To this end, unstimulated Nox2 containing endosomes were isolated from primary mouse dermal fibroblasts (PMDFs), and it was determined whether SOD1 supplementation would activate NADPH-dependent [symbol] O₂ generation by this compartment. To confirm that endosomal [symbol] O₂ was indeed derived from Nox2, PMDFs isolated from Nox2^{gp91phox} KO (−/−) mice or wild type control littermates were used. Iodixanol density gradient separation of vesicular fractions from wild type heavy mitochondrial supernatant demonstrated two predominant peak fractions containing Nox2^{gp91phox}, Rac1, and SOD1 proteins (Fractions # 10 and 12) that overlapped with a small peak in NADPH-dependent [symbol] O₂ production and the early endosomal marker EEA1 (FIG. 3A). Interestingly, Nox2^{gp91phox} KO cells failed to recruit SOD1 to these fractions (FIG. 3B), suggesting that Nox2 must be present in the endosome to facilitate recruitment of SOD1. The addition of purified bovine SOD1 to these isolated endosomes led to a significant enhancement in their ability to produce NADPH-dependent [symbol] O₂ (FIG. 3C). This enhancement in endosomal [symbol] O₂ was sensitive to DPI (an NADPH oxidase inhibitor) and was not observed in Nox2^{gp91phox} KO PMDFs (FIG. 3C), suggesting that the [symbol] O₂ was indeed derived from Nox2. Using a second wild type cell type, primary mouse embryonic fibroblasts (PMEFs), it was confirmed that the addition of exogenous bovine SOD1 to isolated endosomes also enhanced their capacity to produce NADPH-dependent [symbol] O₂ (FIG. 3D). This induction of [symbol] O₂ by PMEFs endosomes was observed with bovine SOD1, but not E. coli SOD (FIG. 3D), despite the equal capacity of both enzymes to dismutate [symbol] O₂ (FIG. 3E).

Intrigued by the ability of SOD1 to enhance production of its substrate ( [symbol] O₂) by Nox2 in endomembranes, it was determined if similar functional effects would be seen in living cells. As a model for Nox2-dependent [symbol] O₂ production, the well-characterized respiratory burst seen following phorbol myristate acetate (PMA) stimulation of polymorphonuclear leukeucytes (PMNs) was used. It was hypothesized that SOD1 deficiency would lead to reduced Nox2 activation and [symbol] O₂ respiratory burst following PMA stimulation. To this end, peripheral blood PMNs were isolated from sod1+/+, sod1+/− or sod1−/− mice and the magnitude of their respiratory burst assessed. Indeed SOD1 deficiency significantly inhibited PMA-induced [symbol] O₂ generation by PMNs (FIG. 3F). Furthermore, PMNs derived from sod1+/− mice exhibited an intermediate reduction in PMA-induced superoxide generation in comparison to sod1+/+ and sod1−/− PMNs (FIG. 3F). Collectively, these results demonstrate that SOD1 can indeed regulate Nox2 activation in vivo and provides a functional context for the ability of SOD1 to regulate Rae. It should be noted that Rac2 is the predominant isoform of Rac in PMNs and immunoprecipitated Rac2 also bound effectively to SOD1 (data not shown).

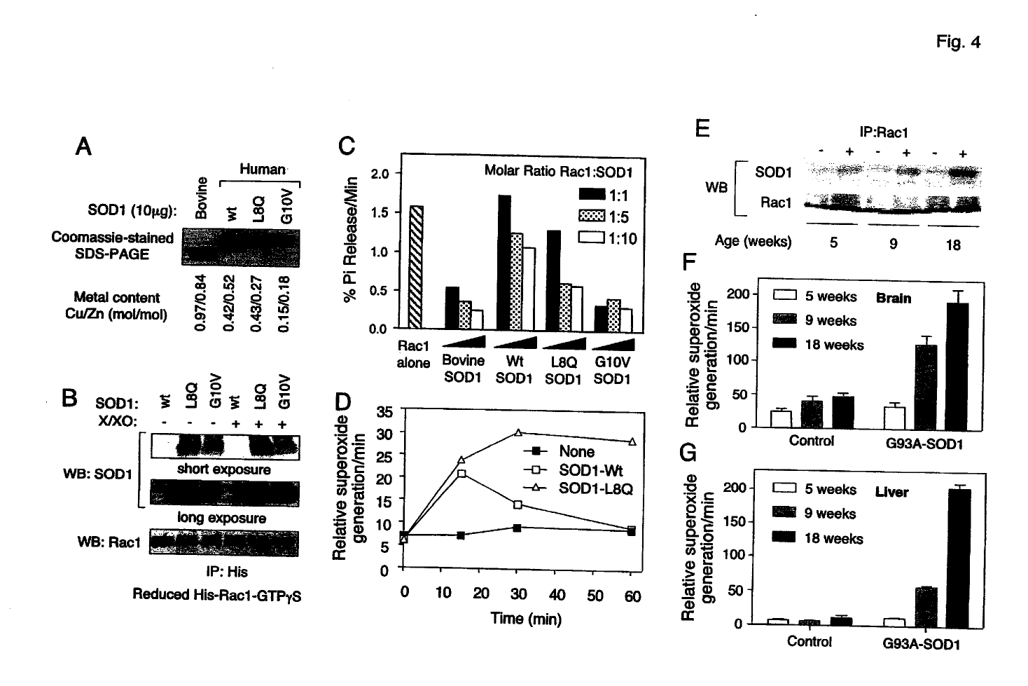

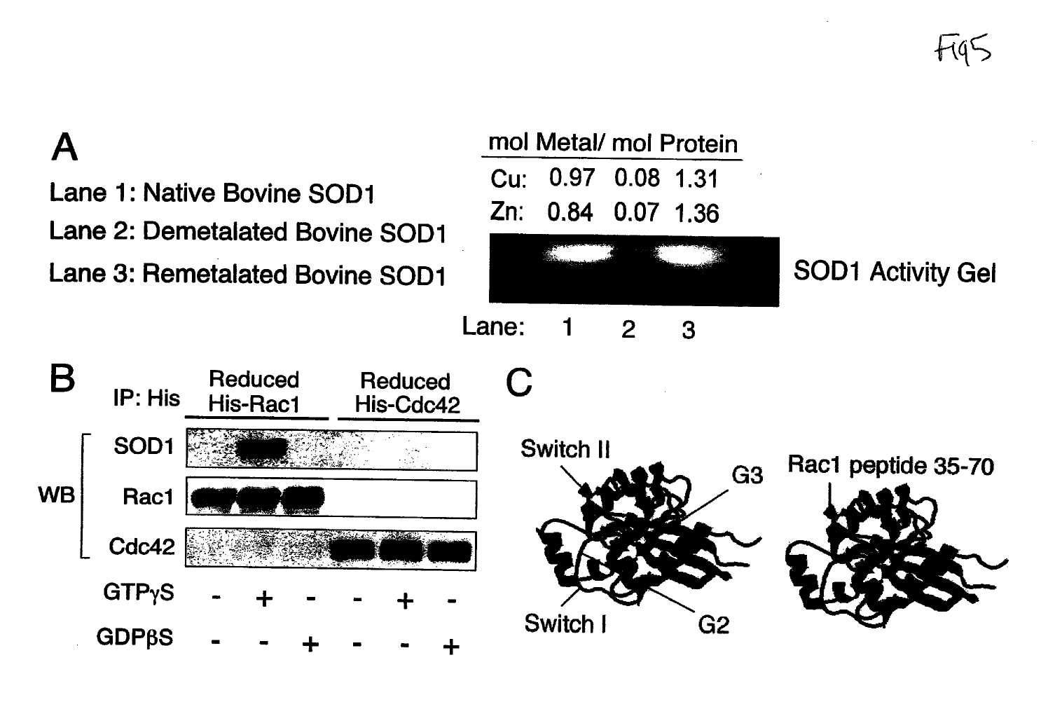

Mutations in SOD1 can lead to a dominant form of inherited amyotrophic lateral sclerosis (ALS), a fatal neurodegenerative disorder associated with progressive loss of motor neurons and subsequent muscle weakness and paralysis (Cleveland et al., 1999). Certain familial forms of dominant ALS caused by SOD1 mutations are thought to promote disease through a toxic gain of function that remains poorly understood. To that end, Rac1 regulation by SOD1 mutants was evaluated. Wild type human SOD1 and two human ALS mutant SOD1 proteins (L8Q and G10V) were expressed in and purified from bacteria (FIG. 4A). Interestingly, both human SOD1 mutant proteins had enhanced ability to bind Rac1 when compared to human wt-SOD1 (FIG. 4B). Unlike human wt-SOD1, binding of these SOD1 mutants to Rac1 was not disrupted by X/XO derived ROS (FIG. 4B), suggesting that the redox-regulation of SOD1/Rac1 interactions is altered by L8Q and G10V mutations in SOD1. Importantly, L8Q and G10V human SOD1 mutant proteins also demonstrated enhanced ability to inhibit Rac1-GTP hydrolysis when compared to human wt-SOD1 (FIG. 4C). Reduced metalation of bacterial-derived human wt-SOD1 (FIG. 4A) led to a decreased effectiveness for inhibiting Rac1-GTPase activity as compared to purified bovine SOD1 (FIG. 4C), which was likely due to reduced binding affinity to Rac1 as shown in FIG. 1D. However, the extent of metalation appeared to have less of an effect on the ability of bacterial-derived human mutant SOD1 proteins to bind Rac1 and inhibit GTPase activity.

Based on the above results, it was hypothesized that certain ALS mutations in SOD1 might dysregulate Nox2 activation in the endosomal compartment by virtue of their more persistent and redoxinsensitive activation of Rac1. To this end, the time course of NADPH-dependent [symbol] O₂ production was evaluated in isolated endosomal fractions following the addition of human wt-SOD1 or L8Q-SOD1 proteins. As previously observed (FIG. 3D), wt-SOD1 activated the production of NADPH-dependent [symbol] O₂ by isolated PMEF endosomes (FIG. 4D). This activation in [symbol] O₂ production peaked by 15 minutes and returned to baseline by 1 hour. Such transient activation is consistent with Nox-derived ROS inhibiting SOD1/Rac1 interactions and activating GTP hydrolysis by Rac1, leading to a self-regulated reduction in Nox activation. In contrast, adding L8Q-SOD1 to PMEF endosomes gave rise to persistent NADPH-dependent [symbol] O₂ production out to 1 hour (FIG. 4D). Collectively, these results suggest that certain ALS mutants of SOD1 are dysregulated in their ability to activate Nox2 by virtue of altered redox sensitive interactions with Rac1.

To confirm that mutations in SOD1 typically associated with ALS also result in elevated NADPH oxidase activity in vivo, a well-characterized G93A-SOD1 transgenic mouse model that produces hind limb paralysis and death by about 18-19 weeks of age was used. As predicted from in vitro association data with L8Q- and G10V-SOD1 mutants (FIG. 4B), SOD1 from G93A-SOD1 transgenic mice more strongly associated with immunoprecipitated Rac1 from brain lysates as compared to transgene negative littermates (FIG. 4E). Interestingly, the association between Rac1 and SOD1 in G93A-SOD1 transgenic mice increased with age and was maximal at the onset of paralysis (about 18 weeks). This increase in Rac1/SOD1 interactions seen in G93A-SOD1 transgenic mice was also paralleled by a significant age-dependent increase in NADPH-dependent superoxide production in total endomembranes isolated from the brain and liver (FIGS. 4F, G). These in vivo data demonstrating enhanced Nox activation in G93A-SOD1 transgenic mice substantiate in vitro findings of enhanced Rac1/Nox activation in the presence of other ALS-associated SOD1 mutants (FIG. 4B-D).

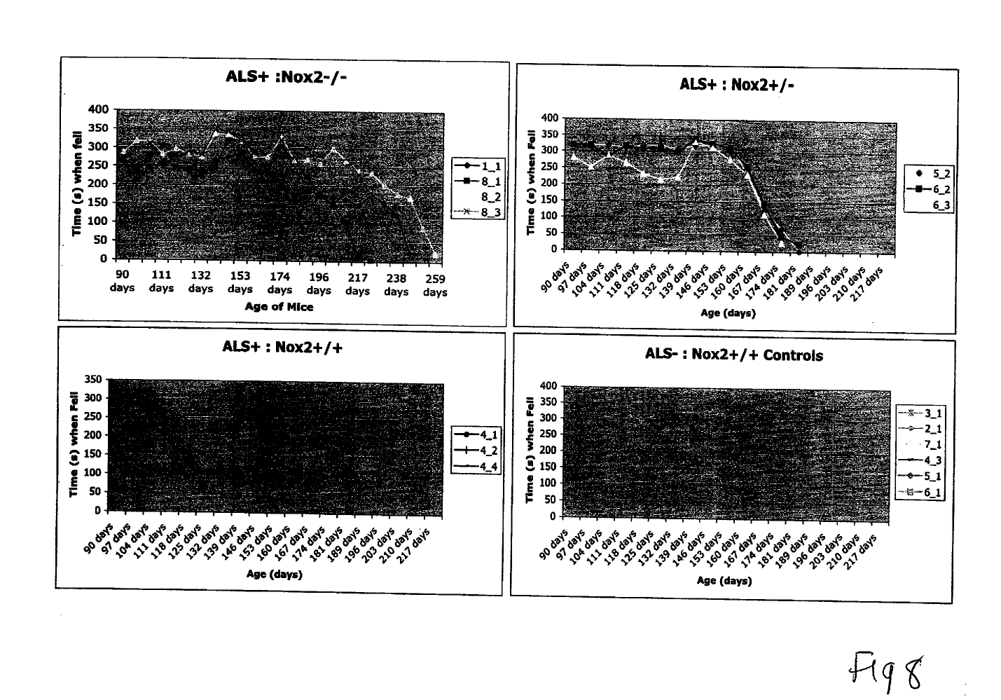

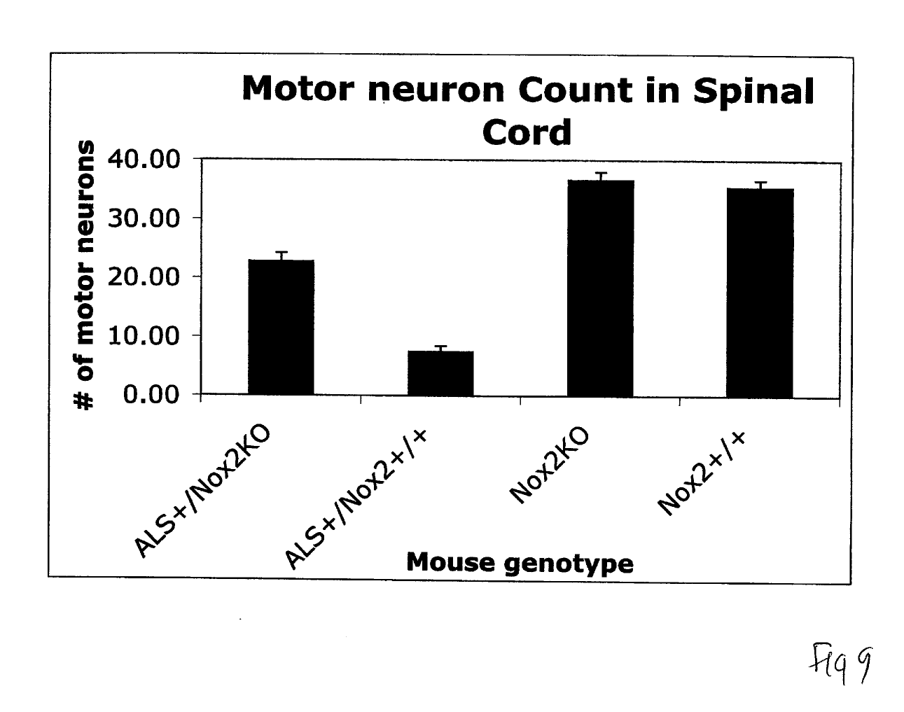

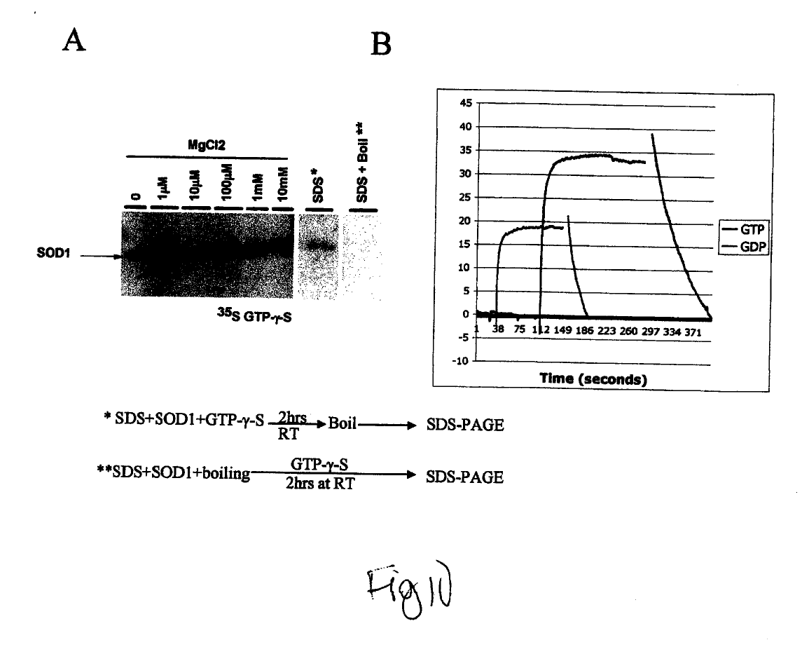

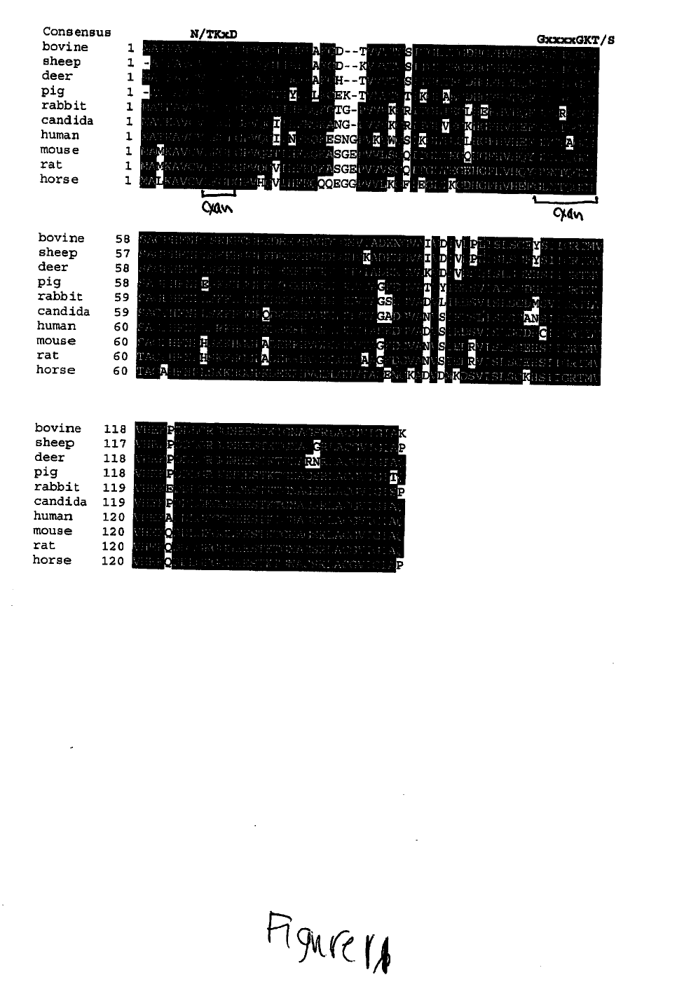

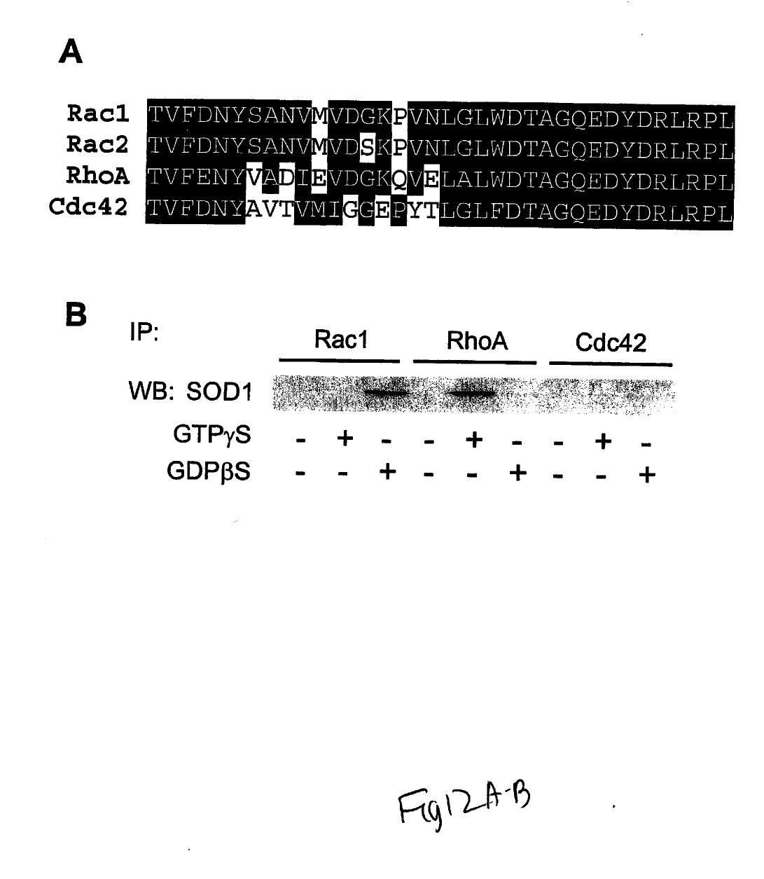

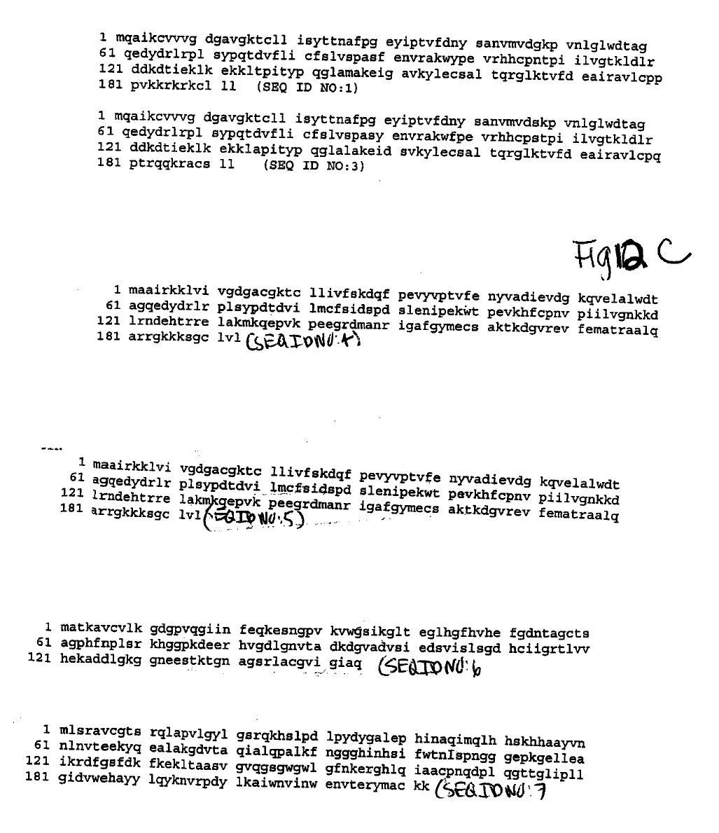

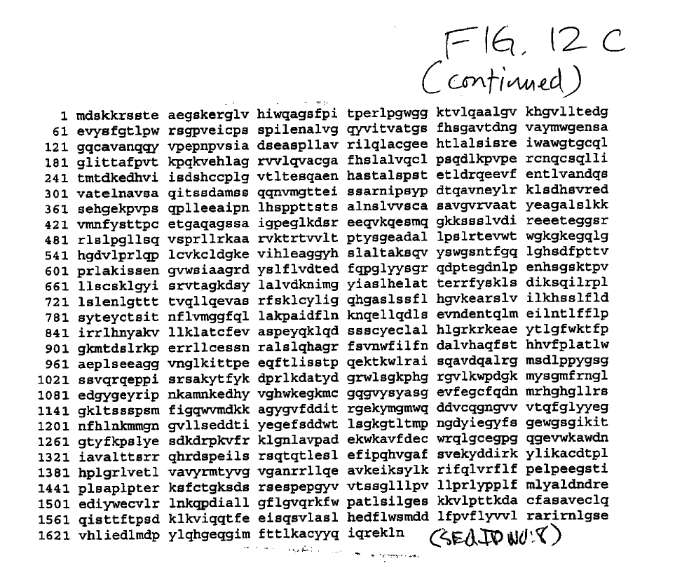

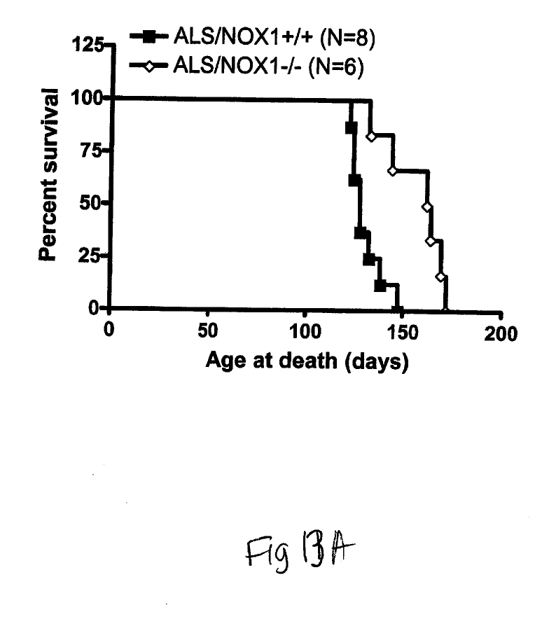

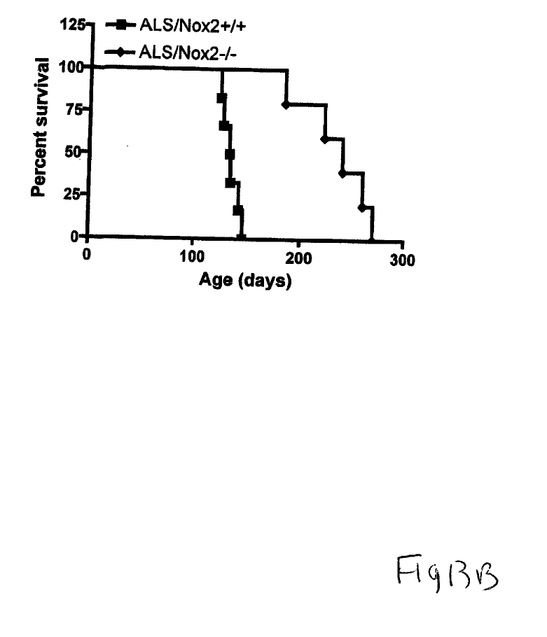

The findings herein demonstrate that SOD1, an enzyme that ubiquitously directs [symbol] O₂→H₂O₂ conversion in cells, has the ability to control Rac1/Nox2 activation through physical interactions with Rac1 in a redox-dependent manner. Additionally, this SOD1-dependent mechanism appears to be conserved for Rac2/Nox2 activation in PMNs. Based on the findings herein, SOD1 may regulate Nox2-dependent [symbol] O₂ production through its ROS-sensitive control of Rac-GTP hydrolysis (FIG. 7). Upon stimulation, activated Rac-GTP is recruited to the assembling membrane associated Nox2 complex along with SOD1. Under the reducing conditions of the cytoplasm, SOD1 efficiently binds to Rac-GTP and inhibits its intrinsic, as well as GAP-facilitated, GTPase activity. This effect results in maintaining Rac in the active state and consequently increases the production of Nox2-derived [symbol] O₂. Local accumulation of H₂O₂ (either by spontaneous or SOD1-facilitated dismutation of [symbol] O₂) leads to the dissociation of SOD1 from Rac-GTP and inactivation of Rac through GTP hydrolysis. Since Rac-GDP cannot support Nox2 activation, this event leads to the inactivation of the Nox2 complex and reduction in ROS production. It is this redox-sensitive uncoupling of SOD1 from Rac that appears to be dysfunctional in certain ALS mutants of SOD1 leading to hyperactivation of Nox-derived [symbol] O₂ by endomembranes. The ability of pM quantities of H₂O₂ to liberate SOD1 from Rac-GTP and allow for GTP hydrolysis to occur, suggests that the mechanism of in vivo regulation may be exquisitely sensitive to small changes in cellular ROS. This mechanism may allow Rac1 to sense spatially related changes in cellular [symbol] O₂ through SOD1 enzymatic conversion to H₂O₂.

These findings may also be of particular importance in neuronal degenerative diseases such as ALS as dysregulation of Rac1/Nox2 activation may contribute to the onset of ALS disease. Interestingly, mutations in the Alsin gene, a recently identified GEF for Rac1 (Topp et al., 2004), have also been shown to lead to recessive forms of ALS (Yang et al., 2001). Hence, there may be a functional link between SOD1 and Alsin mutations responsible for the observed phenotypes that manifest as familial forms of ALS.

### EXAMPLE 2

Materials and Methods

Recombinant expression vectors and siRNA. MCF-7 cells were infected with recombinant adenoviruses (500 particles/cell) as previously described and cells were utilized for experiments at 48 hours post-infection. Lipofectamine™ 2000 (Invitrogen) was used for all plasmid transfections and cells were utilized for experiments at 48 hours post-transfection. The following E1-deleted recombinant adenoviral vectors were used: 1) Ad.GPx-1, which encodes glutathione peroxidase-1 and degrades cytoplasmic H₂O₂ (Duan et al., 1999); 2) Ad.Dyn(DN), which encodes a dominant-negative mutant (K44A) of dynamin and inhibits endocytosis (Li et al., 2001); 3) Ad.NFκBLuc, which encodes an NFκB-responsive promoter driving luciferase expression and was used to assess NFκB transcriptional activation in vivo (Sanglioglu et al., 2001); and 4) Ad.BglII, an empty vector with no insert, was used as a control for viral infection (Li et al., 2001). For NFκB transcriptional assays utilizing infection with two recombinant adenoviruses, a slightly modified sequential infection method was used (Sanglioglu et al., 2001). In this case, cells were infected with experimental vectors (i.e., Ad.Dyn(DN) or Ad.GPx1) 24 hours prior to infection with Ad.NFκBLuc and cells were utilized for experiments at 48 hours post-initial infection. Transduction efficiencies with recombinant adenoviruses were typically 80-90%, as assessed by Ad.CMV-GFP reporter gene expression.

The following plasmids were used for transient transfection experiments: 1) a recombinant plasmid encoding an N-terminal HA-fusion of Rab5 was generated by PCR amplification for immuno-affinity isolation of early endosomes, and 2) an expression plasmid encoding the Nox2 cDNA, a kind gift from Dr. J. D. Lambeth (Emory University).

siRNA against MyD88, Rac1 and Nox2 were obtained from Santa Cruz Biotech and the transfections were preformed using methods and reagents described by the manufacturer. The sequences used for siRNAs were proprietary and not provided by the company.

Cytokine treatments and vesicular isolation. MCF-7 cells were treated with recombinant IL-1β at the indicated concentration for 20 minutes prior to all vesicular isolations. For endosomal loading experiments, purified bovine Cu/ZnSOD (Oxis Research) and/or catalase (Sigma-Aldrich) proteins were added to fresh media (0.1 to 1 mg protein/ml) and applied to cells 10 minutes prior to cytokine treatment in the continued presence of SOD and/or catalase. Cells were washed and scraped into ice-cold PBS. Cell pellets were then resuspended in 0.5 ml of homogenization buffer (0.25 M sucrose, 10 mM triethanolamine, 1 mM EDTA, 1 mM PMSF, and 100 ug/ml aprotinin), homogenized in a Duall tissue grinder (Duall), and centrifuged at 2000×g at 4° C. for 10 minutes. The supernatant was designated the post-nuclear supernatant (PNS). The PNS was subsequently combined with 60% Iodixanol (OptiPrep™, Axis-Shield) solution to obtain a final concentration of 32% and loaded into an sw55Ti centrifuge tube. The PNS was then bottom loaded under two-step gradients of 24% and 20% lodixanol in homogenization buffer. Samples were centrifuged at 30,500 rpm for 2 hours at 4° C. Fractions were collected from the top to the bottom of the centrifuge tube at 4° C. (about 300 μl per fraction) and utilized immediately for NADPH oxidase activity and immuno-isolation, or frozen for Western blot analysis.

NFκB and NADPH oxidase activity assays. NFκB transcriptional activity was assessed using the previously described NFκB-inducible luciferase reporter vector (Ad.NFκBLuc) (Sanglioglu et al., 2001). Luciferase activity was assessed at 6 hr post-cytokine treatment using 5 μg of cell lysate. NADPH oxidase activities were analyzed by measuring the rate of [symbol] O₂ generation using a chemiluminescent, lucigenin-based system (Li et al., 2001). Prior to the initiation of the assay, 5 μg of vesicular proteins were combined with 5 μM lucigenin (Sigma-Aldrich) in PBS and incubated in darkness at room temperature for 10 minutes. The reaction was initiated by the addition of 100 μM of NADPH (Sigma-Aldrich) and changes in luminescence were measured over the course of 3 minutes (5 readings/second). The slope of the luminescence curve (relative light units [RLU] per minute) (r>0.95) was used to calculate the rate of [symbol] O₂ formation as an index of NADPH oxidase activity (RLU/min μg protein). In the absence of NADPH, background levels of lucigenin-dependent luminescence were always >1000-fold less than maximally induced values in the presence of NADPH. Additionally, background levels of luminescence in the absence of NADPH did not significantly vary between samples and had no rate of change.

Electron spin resonance spectroscopy (ESR) was used to confirm the production of NADPH-dependent [symbol] O₂ by isolated endosomes. ESR assays were conducted at room temperature using a Bruker model EMX ESR spectrometer (Bruker). Vesicular fractions from each sample were mixed with the spin-trap, 50 mM DMPO (5,5-dimethyl-1-pyrroline N-oxide), in a total volume of 500 μl of PBS, pH 7.4. This solution contained iminodiacetic acid-chelating resin (10 ml/l) (Sigma-Aldrich). The reaction was initiated by adding NADPH to 100 μM and was immediately placed into the ESR spectrometer. DMPO-hydroxyl radical adduct formation was assayed for 10 minutes. Instrument settings were as follows: receiver gain: 1×10⁶, modulation frequency: 100 kHz, microwave power: 40.14 mW, modulation amplitude: 1.0 G, and sweep rate: 1 G/s.

Vesicular immuno-isolation. Rab5-containing endosomes were isolated based on a previous method (Trischler et al., 1999). Cells were transfected with HA-Rab5a or GFP expression plasmid 48 hours prior to IL-1β treatment. Following iodixanol isolation of intracellular vesicles, one half of the combined peak vesicular fraction was used directly for biochemical analyses, and the other half was used for immuno-affinity isolation using Dynabeads M-500 (Dynal Bioscience) coated with the anti-HA antibody. Prior to use, beads were coated with antibodies as follows: The secondary antibody (anti-rat) was conjugated to Dynabeads (4×10⁸ beads/ml) in 0.1 M of borate buffer (pH 9.5) for 24 hours at 25° C. with slow rocking. The beads were then placed into the magnet for 3 minutes and washed in 0.1% (w/v) BSA/PBS for 5 minutes at 4° C. A final wash in 0.2 M Tris (pH8.5)/BSA was performed for 24 hours. Finally, the beads were resuspended in BSA/PBS and conjugated to 4 μg of primary anti-HA antibody per 10⁷ beads overnight at 4° C. and then washed in BSA/PBS. Vesicular fractions were mixed with 700 μl of coated beads in PBS containing 2 mM EDTA, 5% BSA, and protease inhibitors. The mixture was incubated for 6 hours at 4° C. with slow rocking, followed by magnetic capture and washing in the same tube three times (15 minutes each). Beads with HA-enriched endosomes were then resuspended in PBS, and wash supernatants were saved for analysis.

Western blotting, immunoprecipitations, and in vitro kinase assays. Western blotting was performed using standard protocols (Goligan, 1991), and protein concentrations were determined using the BioRad protein quantification kit. Immunoreactive proteins were detected using enhanced chemiluminescence ECL (Amersham) and were exposed to X-ray film. Antibodies used for Western blotting were as follows: anti-EEA1, anti-HA, anti-Rab5, and anti-Rab11 antibodies (Transduction Laboratories); anti-p47phox, anti-TRAF6, anti-IKKα, anti-Na⁺/K⁺ ATPase(α3), anti-MyD88, and anti-GST antibodies (Santa Cruz Biotech); anti-IL-1R1 (QED Bioscience, Inc.); anti-Cu/ZnSOD and anti-catalase antibodies (Binding Site, Inc.); and anti-mtHSP70 (Affinity Bioreagents). The Nox2 antibody was a kind gift from Dr. A. Jesaitis (Montana State University) (Burritt et al., 1995).

For immunoprecipitations, cells were washed with ice-cold PBS and lysed in RIPA buffer at 4° C. for 30 minutes. 500 μg cellular protein and 5 μl primary antibody were mixed with 1 ml RIPA buffer at 4° C. for 1 hour. 50 μl Protein A-Agarose Beads (Santa Cruz Biotech) were then added to the mixture and rotated for 4 hours. The beads were washed with ice-cold PBS prior to experimental analyses. In vitro kinase assays were performed with immunoprecipitated IKKα and/or isolated vesicles using GST-IκBα as a substrate. Kinase reactions were performed with 1 μg GST-IκBα, 0.3 mM cold ATP, and 10 μCi [γ-³²P]ATP in 10 μl kinase buffer (40 mM Hepes, 1 mM β-glycerophosphate, 1 mM Nitrophenolphosphate, 1 mM Na₃VO₄, 10 mM MgCl₂, and 2 mM DTT). The reactions were then incubated at 30° C. for 30 minutes. Reactions terminated by the addition of SDS-PAGE protein-loading buffer and boiled at 98° C. for 5 minutes. Following SDS-PAGE, gels were transferred to nitrocellulose membranes and exposed to X-ray film prior to probing with an anti-GST antibody.

In vivo localization of redox-active endosomes and ROS production. In vivo localization of superoxides within endosomes was performed using OxyBURST Green H₂HFF-BSA (Molecular Probes). Stock solutions (1 mg/ml) were generated immediately prior to use by dissolving H₂HFF-BSA in PBS under nitrogen and protected from light. Cells were incubated in the presence of 50 μg/ml OxyBurst Green H₂HFF-BSA for 2 minutes at 37° C. and then stimulated by the addition of IL-1β (1 ng/ml). Cells were fixed in 4% paraformaldehyde at various times (1-10 minutes) post-stimulation and evaluated by fluorescent microscopy. Various compounds (1 mg/ml SOD or 10 μM DPI) were added at the time of IL-1β stimulation. Co-localization of H₂HFF-BSA and EEA1 was performed by immunofluorescent localization in post-fixed samples using an anti-EEA1 monoclonal antibody (Transduction Laboratories) and a Texas Red-Conjugated Goat Anti-Mouse Antibody (Jackson ImmunoResearch Laboratories). In vivo localization of total cellular ROS (predominantly H₂O₂) was performed using H₂DCFDA (Molecular Probes). Stock solutions of H₂DCFDA were generated in DMSO at a concentration of 50 μg/ml immediately prior to use. Cells were washed 3 times with PBS prior to the simultaneous treatment with H₂DCFDA (10 μM) and IL-1β (1 ng/ml) for 20 minutes in PBS at 37° C. in the dark. For samples infected with adenoviral vectors, this was done 48 hrs prior to stimulating with IL-1β. When SOD/Catalase proteins were added to media, this was done at a concentration of 1 mg/ml at the time of IL-1β stimulation. Following washing and fixation for 10 minutes in 4% paraformaldehyde, cells were mounted in DAPI containing antifadent and examined by fluorescent microscopy for DCF signal.

Results

Endocytosis and endosomal ROS play key roles in IL-1β-mediated NFκB activation. IL-1β induction of NFκB was evaluated in an epithelial cell line (MCF-7) as a model for studying redox-sensitive signal transduction. This model demonstrated that IL-1β induction of a transcriptional NFκB luciferase reporter was significantly inhibited (about 50%) by recombinant adenoviral-mediated over expression of GPx1 (which degrades H₂O₂→H₂O in the cytoplasm). In these studies, approximately 85% of cells were transduced with recombinant adenovirus as determined using a GFP reporter. Similarly, partial inhibition of endocytosis by over expression of dominant negative dynamin K44A (Ad.Dyn(DN)) (Conner et al., 2003) also inhibited NFκB to a similar extent. These findings suggested that ROS production and endocytosis were equally required for a significant fraction of NFκB activation by IL-1β.

Endocytosis of ligand-bound receptors is often intricately linked to the processing and propagation of intracellular signals (Sorkin et al., 2002). However, the potential links between receptor processing and redox-dependent activation in the endosome have not been previously investigated. Based on the results, it was hypothesized that endosomal-derived ROS production following IL-1β stimulation might be responsible for amplifying receptor/effector activation through a redox-dependent process. To this end, it was investigated whether ROS clearance from the endosomal compartment might also influence NFκB activation. Purified Cu/ZnSOD and catalase proteins were efficiently taken up by MCF-7 cells when added to the media at 1 mg/ml concentration. Indeed, cellular uptake of Cu/ZnSOD and catalase by MCF-7 cells significantly reduced both IKK and NFκB activation by IL-1β in a dose dependent fashion. The synergistic ability of Cu/ZnSOD and catalase to inhibit IKK and NFκB activation together, more effectively than either alone, suggested that both endosomal [symbol] O₂ and H₂O₂ were likely involved in IL-1R1 complex activation. Furthermore, overexpression of cytoplasmic GPx-1 also inhibited NFκB activation and suggested that H₂O₂ was a likely redox-second messenger of the NFκB pathway. To confirm that GPx-1 expression and cellular loading with Cu/ZnSOD/catalase both reduced cellular ROS following IL-1β treatment, a ROS-sensitive dye (H₂DCFDA) was used to assess the level of cellular ROS under the various treatment conditions. IL-1β treatment stimulated cellular ROS, and expression of GPx-1 or cellular loading with Cu/ZnSOD/catalase both inhibited DCF fluorescence. These findings led to the investigation of the mechanism of ROS generation within the endosomal compartment, and how such ROS might influence the IL-1R1 complex to become competent for IKK complex activation.

IL-1β stimulates endosomal NADPH-dependent .O₂ production required for TRAF6 recruitment. It was hypothesized that Nox complexes within ligand-activated endosomes might serve as sources of the ROS required for IL-1β-mediated NFκB activation. To this end, it was determined whether IL-1β could stimulate NADPH-dependent [symbol] O₂ production in vesicular fractions of MCF-7 cells. Peak vesicular fractions isolated by lodixanol density gradient centrifugation expressed Rab5 and Rab11, two vesicular markers of early and recycling endosomes, respectively (Zerial et al., 2001). They also contained internalized biotin-transferrin, as would be expected for this compartment. However, vesicular fractions were devoid of mitochondrial mtHSP70, plasma membrane Na⁺/K⁺-ATPase, or peroxisomal catalase, demonstrating little, if any, contamination from these compartments. In contrast, peak Rab5/11 vesicular fractions demonstrated significant overlap with ER, golgi, and lysosomal enzymes, as would be expected from this isolation strategy.

Using a lucigenin-based chemiluminescence assay to detect [symbol] O₂ production in the various Iodixanol fractions, the rate of NADPH-dependent [symbol] O₂ production was assessed as an index of Nox activity. As hypothesized, IL-1β stimulation significantly increased NADPH-dependent [symbol] O₂ production in peak vesicular fractions #3-4. Having established that IL-1β induces the formation of NADPH oxidase-active endosomes, it was next sought to establish whether [symbol] O₂ was generated in the lumen of isolated IL-1β-activated endosomes, as predicted by the ability of endocytosed ROS scavenging enzymes to inhibit IKK/NFκB activation. To address this question, the ability of Cu/ZnSOD protein in the media to be taken up into endosomes and degrade [symbol] O₂ from the interior of isolated endosomes was evaluated. Since lucigenin, but not Cu/ZnSOD protein, is membrane permeable, the extent to which [symbol] O₂ was produced in the interior of endosomes could be determined using the lucigenin-based chemiluminescence assay. Biochemical studies confirmed that when the Cu/ZnSOD protein was added to the media [SOD(m)], it was indeed internalized into isolated endosomes and remained resistant to pronase digestion. In contrast, Cu/ZnSOD added to the exterior of unloaded isolated endosomes [SOD(v)] was effectively degraded by pronase. As expected, disruption of endosomal membranes with Triton-X-100 sensitized intra-lumenal Cu/ZnSOD [SOD(m)] to pronase degradation. Hence, Cu/ZnSOD protein in the media is indeed taken up into the endosomal compartment. An unexpected finding from these bovine Cu/ZnSOD (bSOD) endosomal-loading experiments was the IL-1β-dependent recruitment of endogenous cellular human Cu/ZnSOD (hSOD) protein to vesicular membranes. This cellular human Cu/ZnSOD was sensitive to pronase digestion in the absence of Triton-X-100, demonstrating that it resided on the endosomal surface. These findings suggest the intriguing possibility that Cu/ZnSOD may play an active role in ROS metabolism at the endosomal level following IL-1β stimulation.

The ability of intra-lumenal Cu/ZnSOD to inhibit IL-1β-induced [symbol] O₂ production by isolated vesicles suggested that the majority of [symbol] O₂ were generated from within the interior of isolated endosomes. The addition of KCN (Cu/ZnSOD inhibitor) to the lucigenin reaction completely reversed this inhibition and demonstrated that the inhibitory effect was specifically due to enzymatic Cu/ZnSOD activity. Enhanced NADPH-dependent production of [symbol] O₂ by IL-1β-activated endosomes was also confirmed using electron spin resonance spectroscopy (ESR). In this context, DMPO adduct formation was completely inhibited by endosomal loading with Cu/ZnSOD, but not catalase, prior to vesicular isolation and ESR analysis. This finding demonstrated that [symbol] O₂, not H₂O₂, was the predominant ROS leading to ESR signal. NADPH-dependent [symbol] O₂ production in peak vesicular fractions was also sensitive to diphenylene iodonium (DPI) (a NADPH oxidase inhibitor), but not to rotenone or antimycin A (specific inhibitors of mitochondrial electron transport chain complex I or III, respectively). These findings ruled out significant mitochondrial contamination as the source of ROS generation in the vesicular fractions. IL-1β stimulation of endosomal [symbol] O₂ was also dependent on endocytosis, as demonstrated by a 75% reduction in the presence of Ad.Dyn(DN) infection, but not following infection with an empty vector control adenovirus. Such a reduction closely mirrored the extent of inhibition of transferrin uptake following Ad.Dyn(DN) infection. Cumulatively, these studies and the fact that endosomal loading with Cu/ZnSOD/Catalase significantly reduced IL-1β stimulated DCF fluorescence, suggest that IL-1β induces [symbol] O₂ and H₂O₂ production in MCF-7 cells predominantly within an endosomal compartment following receptor endocytosis.

Given the ability of ROS clearance from inside the endosomal compartment to inhibit IKK and NFκB activation by IL-1β, it was hypothesized that redox-active endosomes might provide the subcellular framework for spatially controlled redox-dependent activation of the IL-1R complex. MyD88 is well recognized as one of the first effectors recruited to IL-1R1 following ligand binding. This process stimulates an ordered recruitment of effectors and adaptors (IL-1R1→MyD88→IRAK→TRAF6), which ultimately leads to the formation of an active IKK kinase complex capable of activating NFκB (Ghosh et al., 2002). Using endosomal loading with SOD and catalase, the redox dependence of MyD88 and TRAF6 recruitment to the endosomal compartment following IL-1β stimulation was evaluated in purified vesicular fractions. Results from these experiments demonstrated that TRAF6 recruitment to the endosomal compartment following IL-1β stimulation was reduced about 50% by endosomal loading of SOD/catalase, a finding which closely mirrored the reduction in total cellular IKK kinase activity under similar conditions. In contrast, endosomal loading of ROS clearance enzymes did not alter MyD88 recruitment to the endosomal fraction following IL-1β stimulation. These findings suggested the recruitment of TRAF6 to IL-1R1 might occur in a redox-dependent fashion at the level of the endosome.

IL-1β induces Nox2 complex activation in the endosomal compartment. Having established that IL-1β induces [symbol] O₂ production by the endosomal compartment in a NADPH-dependent fashion, a candidate Nox enzyme(s) that might be responsible for endosomal [symbol] O₂ production was identified. Since Nox activation in the endosomal compartment was largely dependent on endocytosis, it was hypothesized that specific subunits of the NADPH-oxidase complex would likely be recruited into endosomes following ligand stimulation. RT-PCR analysis for Nox1, 2, 3, 4, 5 mRNA in MCF-7 cells demonstrated that only Nox2 and Nox5 mRNA expression could be detected in this cell line. Subsequent analysis of purified endosomes demonstrated that IL-1β stimulation promoted the recruitment of three known Nox2 activators (Rac1, p67phox, and p47phox) to endomembranes. Furthermore, inhibiting endocytosis through the expression of dynamin(K44A) [i.e., following Ad.Dyn(DN) infection], significantly attenuated IL-1β-mediated recruitment of Rac1, p67phox, and p47phox to the vesicular fraction. These findings suggested that membrane internalization following IL-1β stimulation was required for the formation of an active endosomal Nox complex. They also substantiated earlier findings that endocytosis was required for IL-1β induction of [symbol] O₂ by the endosomal compartment.

Next it was evaluated how endosomal [symbol] O₂ and/or H₂O₂ might influence the recruitment of various IL-1R1 (MyD88, TRAF6) or Nox (Rac1, p67phox, p47phox) effectors to the endosomal compartment following ligand stimulation. To this end, endosomes were loaded at the time of IL-1β stimulation by the addition of SOD or SOD/Catalase to the media and evaluated the recruitment of these various effectors to isolated endosomes. Results from these experiments demonstrated that only the SOD/Catalase combination inhibited recruitment of TRAF6 to endosomes following IL-1β stimulation. The lack of a functional effect with SOD loading alone, suggested that H₂O₂ is the primary ROS effector required for the recruitment of TRAF6 to the endosome. In contrast, the recruitment of MyD88, Rac1, p67phox, or p47phox to IL-1β-activated endosomes remained unaffected by SOD or SOD/Catalase loading. Kinetic analysis of IL-1R1, Nox2, MyD88 and TRAF6 recruitment into endosomes demonstrated that maximal endocytosis of IL-1R1 occurred by 15-30 minutes following IL-1 treatment, concordant with MyD88 recruitment. TRAF6 recruitment to endosomes lagged maximal levels of Nox2 in the endosomal compartment, as expected if Nox2-derived ROS was required to facilitate TRAF6 binding to the IL-1R1 endosomal complex. Interestingly, Nox2 was cleared more rapidly from the endosomal compartment than IL-1R1 suggesting that endosomal processing removes Nox2 after maximal recruitment of TRAF6 has occurred. Loading of SOD/Catalase reduced TRAF6 recruitment to endosomes at all time points, but did not affect IL-1R1, Nox2, or MyD88 levels in the endosome. Cumulatively, these studies suggest that activation of endosomal Nox complexes following IL-1β stimulation is dependent on endocytosis from the plasma membrane, and that this process influences the redox-dependent recruitment of TRAF6 to its endosomal ligand-activated receptor complex.

Rac1, p67phox, and p47phox have all been associated as co-activators of Nox1 and 2, but not Nox3, 4, or 5 (Lambeth et al., 2004; Park et al., 2004). Given the fact that only Nox5 and Nox2 mRNA expression was detected in MCF-7 cells, Nox2 might be responsible for ROS production by the endosomal compartment following IL-1β stimulation. Two approaches were used to address this hypothesis. The first approach involved attempting to modulate endosomal ROS production by ectopically overexpressing Nox2 using transient transfection. Ectopic expression of Nox2 significantly enhanced NADPH-dependent [symbol] O₂ production by isolated IL-1β-stimulated endosomes in comparison to transfection with an irrelevant pcDNA plasmid. Furthermore, overexpression of Nox2 significantly enhanced Nox2 incorporation into endosomes only following IL-1β-stimulation. Although the levels of endogenous Nox2 were extremely low in MCF-7 cells, these studies also demonstrated enhanced recruitment of endogenous Nox2 to endosomes only following IL-1β stimulation. Using a second approach, it was demonstrated that Nox2 siRNA, but not an irrelevant scrambled siRNA, significantly inhibited Nox2 protein expression in MCF-7 cells and NADPH-dependent [symbol] O₂ production by isolated IL-1β-stimulated endosomes. Furthermore, Nox2 siRNA significantly reduced recruitment of both ectopically expressed and endogenous Nox2 to the endosomal compartment following IL-1β-stimulation. Nox2 siRNA, but not scrambled siRNA, also attenuated IL-1β-induced NFκB transcriptional activation and endosomal NADPH-dependent superoxide production to similar extents. Cumulatively, these studies provide strong molecular and functional confirmation that Nox2 complexes are activated in IL-1β-stimulated endosomes.

IL-1β induces Nox2 complex activation in early endosomes. Based on the finding that ligand-stimulated endocytosis was required for Nox2 activation in the endosomal compartment, it was next hypothesized that the formation of these redox-active endosomes likely initiated at the level of the early endosome. To investigate this hypothesis, ROS production in the early endosomal compartment was probed using a membrane-impermeable BSA-conjugated fluorescent dye dihydro-2′,4,5,6,7,7′-hexafluorofluorescein (H₂HFF-BSA). By incubating cells in the presence of H₂HFF-BSA, the endosomal compartment was loaded with this dye and [symbol] O₂ detected by a green fluorescence signal. This study demonstrated a dramatic increase in the H₂HFF-BSA endosomal fluorescence following IL-1β treatment for 10 minutes. IL-1β-induced H₂HFF-BSA fluorescence was significantly inhibited by treating cells with DPI or loading purified Cu/ZnSOD protein into the endosomal compartment. These findings confirmed that Nox-derived [symbol] O₂ were the major ROS detected by H₂HFF-BSA in the endosomal compartment. Co-localization studies with H₂HFF-BSA and Early Endosomal Antigen-1 (EEA1) demonstrated that IL-1β significantly increased the abundance of EEA1 and H₂HFF-BSA co-positive endosomes as compared to unstimulated cells. Additionally, IL-1β stimulation led to an increase in H₂HFF-BSA-positive endosomes that did not contain EEA1; however, this population was less abundant at early time points post-stimulation and increased with time. These findings are consistent with the notion that the ligand-stimulated [symbol] O₂-producing redox-active endosomes are originated in the EEA1 compartment, while retaining some ability to produce [symbol] O₂ after being processed into downstream endosomal compartments.

To provide additional biochemical confirmation for redox-active endosome formation in the early endosomal compartment following IL-1β stimulation, early Rab5-positive endosomes were purified using an immuno-affinity isolation procedure. Rab5, an early endosome-specific GTPase, plays a critical role in trafficking and membrane fusion of the early endosome. Purification of this compartment was facilitated by the overexpression of a recombinant HA-tagged Rab5 and immuno-affinity isolation from lodixanol-isolated endosomes using anti-HA antibodies linked to Dynabeads. Results from these immuno-affinity isolation experiments demonstrated that a significant portion of Nox activity (i.e., NADPH-dependent [symbol] O₂ production) was associated with the HA-Rab5 compartment (Dynabead pellet) following IL-1β-stimulation. This activity represented approximately ⅓ of the total NADPH oxidase activity in the starting fraction. The specificity of this isolation procedure was confirmed by several criteria. First, no significant contamination of Rab11 recycling endosomes was seen in the purified Rab5 endosomal fractions. Second, Dynabeads coated with the secondary antibody alone, or isolated with both 1° and 2° antibodies from control GFP-transfected cells, demonstrated only low background levels of Nox activity associated with the beads. The integrity of Rab5-isolated endosomes was also confirmed by the retention of intravesicular biotin-transferrin loaded at the time of IL-1β treatment. Considering the efficiency of the HA affinity-isolation (about 75%), these results suggested that at least half of the redox-active endosomes were Rab5-associated early endosomes at the time point evaluated (20 minutes). Given the fact that the Rab5 compartment is the earliest endosomal compartment to form following receptor endocytosis, these studies also support the hypothesis that Nox2 is recruited from the plasma membrane into the redox-active endosomes.

Rac1 and MyD88 both control the formation of redox-active endosomes, TRAF6 recruitment to IL-1R1, and NFκB activation following IL-1β stimulation. The data thus far has demonstrated that IL-1β stimulation leads to the formation of redox-active endosomes containing Nox2 complex subunits (Nox2, Rac1, p47phox, and p67phox). ROS generation by these Nox2-active endosomes was critical for the recruitment of TRAF6, but not MyD88, to vesicular membranes. Given that Nox2 activation in the endosomal compartment required active endocytosis, it was reasoned that internalization of IL-1β bound IL-1R1 coordinates the recruitment of the Nox2 catalytic subunit into the endosome. However, currently there are no reports describing the molecular determinants for IL-1R1 internalization following ligand binding. For example, although MyD88 is known to be one of the first effectors to recruit to IL-1R1 following ligand binding and is essential for NFκB activation by IL-1R1 (Akira et al., 2003), it is unclear if MyD88 is essential for receptor internalization following ligand binding. Furthermore, previous studies have suggested that Rac1 associates with the IL-1R1 complex through an interaction with MyD88 (Jeffries et al., 2001). Since Rac1 is known to be part of the active Nox2 complex, it was reasoned that Rac1 might recruit the Nox2 into IL-1R1 containing endosomes through its interaction with the receptor complex at the plasma membrane. The present findings, demonstrating that IL-1β stimulation promotes [symbol] O₂ production in EEA1/Rab5 positive early endosomes, also support the hypothesis that Nox2 (an integral membrane protein) enters the endosomal compartment very early from the plasma membrane.

To investigate the contribution of MyD88 and Rac1 in the internalization of IL-1R1 and the formation of redox-active endosomes, RNA inhibition (RNAi) strategies to inhibit both MyD88 and Rac1 expression were pursued. Transfection of siRNA targeting either MyD88 or Rac1 effectively inhibited their expression at the protein level. Such inhibition was not observed with a scrambled siRNA control. As predicted from previous studies in MyD88 deficient cells (Akira et al., 2003), NFκB activation was significantly inhibited by MyD88 siRNA. Interestingly, Rac1 siRNA also inhibited NFκB activation to a similar extent as seen with MyD88 siRNA. However, simultaneous transfection of both MyD88 and Rac1 siRNA did not provide additive inhibition of NFκB, as compared to either siRNA alone, suggesting that the two factors act on the same pathway to activate NFκB by IL-1β. Furthermore, MyD88 or Rac1 siRNA inhibited [symbol] O₂ production by the endosomal compartment following IL-1β challenge; however, Rac1 siRNA provided a slightly greater level of inhibition. These findings suggested that both MyD88 and Rac1 were critical for NFκB activation and Nox2 activation in the endosomal compartment following IL-1β stimulation.

Next it was investigated whether Rac1 indeed associated with IL-1R1, and if so, whether this interaction was dependent on MyD88. Indeed, it was observed that Rac1 does associate with immunoprecipitated IL-1R1 following ligand simulation. However, in contrast to previous reports suggesting that Rac1 association with the IL-1R1 complex was dependent on MyD88 (Jeffries et al., 2001), very little reduction in Rac1 association with IL-1R1 was observed when MyD88 levels were significantly reduced by RNAi. Similarly, Rac1 siRNA reduced Rac1, but not MyD88, association with IL-1R1. These findings suggest that MyD88 and Rac1 associate independently with IL-1R1 following ligand stimulation. However, RNAi inhibition of either MyD88 or Rac1 abrogated TRAF6 recruitment to the receptor complex. This finding is consistent with the fact that RNAi against MyD88 or Rac1 inhibited the formation of redox-active endosomes and NFκB activation. Given the fact that endosomal ROS was important for TRAF6 recruitment to endosomes following IL-1β stimulation, these studies suggest that MyD88 and Rac1 are two critical factors involved in the formation of redox-activate endosomes, an event required for the redox-dependent recruitment of TRAF6 to IL-1R1 and NFκB activation.

To determine the roles MyD88 and Rac1 play in the formation of redox-active endosomes, the contributions of these two factors on internalization of the receptor and Nox2 into redox-active endosomes was dissected. It was reasoned that MyD88 played a major role in initiating endocytosis of the receptor following ligand binding, while Rac1 was responsible for recruiting Nox2 into endosomes harboring the ligand-bound receptor. MCF-7 cells were transfected with MyD88 or Rac1 siRNA, and the recruitment of IL-1R1, MyD88, TRAF6, Rac1, and Nox2 into the endomembrane fraction was evaluated by Western blotting. Findings from these studies demonstrated that MyD88 inhibition by RNAi significantly attenuated internalization of IL-1R1 and the recruitment of MyD88, TRAF6, Rac1, and Nox2 to endomembranes. These findings suggest that the inhibition of MyD88 abrogates the formation of redox-active endosomes following IL-1β stimulation in a similar fashion to dynaminK44A, by preventing receptor-mediated endocytosis of Rac1/Nox2 complexes into the endosomal compartment. In contrast to MyD88 siRNA, Rac1 siRNA did not inhibit IL-1R1/MyD88 internalization following ligand stimulation, but rather significantly inhibited the recruitment of Rac1, Nox2, and TRAF6 to the endosomal compartment. These findings, together with the redox-dependency of TRAF6 recruitment to the endosomal compartment, suggest that Rac1 plays a critical role in recruiting TRAF6 to endosomal ligand-activated IL-1R1 by facilitating the recruitment/activation of Nox2 in the endosomal compartment. Cumulatively, these studies indicate that both MyD88 and Rac1 play critical roles in establishing the formation of redox-active endosomes by coordinating endocytosis of the receptor and recruitment of Nox2, respectively. Both processes are important for effective recruitment of TRAF6 to the ligand-activated IL-1R1 in the endosomal compartment and IKK/NFκB activation following IL-1β stimulation.

MyD88 binds to IL-1R1 at the plasma membrane while TRAF6 is recruited to endosomal IL-1R1 in an H₂O₂-dependent fashion. These findings demonstrate for the first time that MyD88 is essential for IL-1R1 internalization into the endosomal compartment and suggest that MyD88 is recruited to the plasma membrane following ligand binding and prior to receptor internalization. Furthermore, recruitment of MyD88 to IL-1β activated endosomes was not dependent on the endosomal redox state. In contrast, our studies demonstrate that TRAF6 recruitment to IL-1β activated endosomes was dependent on ROS production by the endosomal compartment. These findings suggested that IL-1R1 recruitment of TRAF6 might occur in a redox-dependent fashion at the level of the endosome. Furthermore, since both catalase and SOD endosomal loading were required to efficiently block IL-1β-mediated TRAF6 endosomal recruitment and IKK activation, it was hypothesized that Nox2-derived H₂O₂ was necessary for the recruitment of TRAF6 to the endosome. To investigate this hypothesis, the extent to which MyD88 and TRAF6 were recruited to IL-1R1 in the plasma membrane and endosomal compartments following ligand binding, and the extent to which these processes were dependent on H₂O₂, were evaluated.

To evaluate the recruitment of MyD88 and TRAF6 to IL-1R1 in the plasma membrane, experiments were performed under conditions in which endocytosis was blocked (at 4° C.) or significantly inhibited by dynamin(K44A) expression. Results from these experiments confirmed that inhibiting endocytosis significantly impaired TRAF6, but not MyD88, recruitment to immunoprecipitated ligand-activated IL-1R1. For example, in the absence of endocytosis at 4° C., TRAF6 was unable to bind to IL-1R1 following IL-1 stimulation, while MyD88 binding was similar to that seen at 37° C. Interestingly, the redox-dependent recruitment of TRAF6-IL-1R1 could be reconstituted at the plasma membrane in the absence of endocytosis by the addition of exogenous H₂O₂; 500 μM H₂O₂ effectively promoted recruitment of TRAF6 to only ligand-activated IL-1R1 at the plasma membrane at 4° C. Such findings provide new insights into several aspects of IL-1R1 activation. First, they demonstrate that TRAF6 effector recruitment to ligand-activated IL-1R1 predominantly occurs at the level of the endosome. Second, they demonstrate that H₂O₂ is likely the ROS that facilitates TRAF6 recruitment to ligand-activated IL-1R1. Third, they provide a physiologic framework for Nox2 activation in endosomes as the source of H₂O₂ for this recruitment process.

Endosomal ROS enhances IL-1β-dependent activation of IKK by the endosomal compartment. Ligand activation of IL-1R1 facilitates IKK activation through the recruitment of at least two potential IKK kinases (TAK1 and/or NIK) to its receptor-associated effector complex (Ghosh et al., 2002). Once the IKK complex is phosphorylated by the activated receptor complex, IKK is activated to phosphorylate IκBα/β, and NFκB is mobilized to the nucleus. To better understand how redox-active endosomes functionally regulate NFκB activation, we next investigated whether isolated IL-1β-stimulated endosomes could directly activate the IKK complex. This in vitro reconstitution assay utilized isolated vesicular fractions and immunoprecipitated IKK complex as kinase activation sources, and phosphorylation of GST-IκBα as the molecular marker of IKK activation. First, it was confirmed that endosomes isolated from the IL-1β-treated cells could activate immunoprecipitated IKK complex from naive cells. Immunoprecipitated IKK complex from non-IL-1β-treated cells was activated to phosphorylate GST-IκBα in the presence of IL-1β-activated endosomes. No activation was seen in the presence of unstimulated endosomes. Moreover, loading of both SOD and catalase into IL-1β-activated endosomes significantly inhibited their ability to activate IKK, while SOD loading alone had little effect. These findings provide direct evidence for the importance of endosomal derived ROS in the activation of IKK, and are consistent with H₂O₂ being the primary ROS required for TRAF6 recruitment to the receptor complex. Similarly, expression of dynamin(K44A) also inhibited vesicular IKK activation, as would be expected since dynamin(K44A) inhibited the formation of redox-active endosomes and recruitment of TRAF6 to IL-1R1. Interestingly, a low level of GST-IκBα phosphorylation was observed with IL-1-activated endosomes in the absence of immunoprecipitated naive IKK complex. This finding suggests that the IKK complex may only transiently associate with the activated receptor complex on redox-active endosomes. Such a finding is similar to IκBα/IKK complex interactions, which demonstrate that IκBα dissociates from the IKK complex once it is phosphorylated on S42/S46 (Regnier et al., 1997).

Discussion

Endocytosis has long been regarded as a classical mechanism for down-regulating receptor-mediated signaling at the plasma membrane. However, increasing evidence has indicated that endocytosis also plays an important role in the activation, amplification, and sorting of membrane-initiated receptor signals (Sorkin et al., 2002). Here, a new redox-dependent mechanism of receptor activation linked to Nox2 activation and ROS production by the early endosomal compartment is described. The identification of Nox2-active endosomes following IL-1β stimulation provided a framework for understanding how ROS can influence IL-1 receptor activation of NFκB. Although the concept of ROS involvement in the activation of NFκB remains controversial (Hayakawa et al., 2003), several reports have implicated H₂O₂ as a key mediator in IL-1β and TNFα activation of NFκB by demonstrating inhibition with over-expressed glutathione peroxidase (Kretz-Remy et al., 1996; Li et al., 2001). Findings from the present study have elucidated the series of events that control IL-1R1 endocytosis following ligand binding and the subsequent H₂O₂-dependent recruitment of TRAF6 to the MyD88/IL-1R1 complex in the endosomal compartment. This redox-dependent process was necessary for efficient activation of the IKK complex and NFκB.

The studies have focused on determining the molecular events that control Nox2 activation in the endosomal compartment following IL-1β stimulation. In this regard, endocytosis of ligand activated IL-1R1 was necessary for efficient Nox2 complex activation and production of ROS by the endosomal compartment. This process was a major controlling event responsible for the redox-dependent recruitment of TRAF6 to ligand-activated endosomal IL-1R1 effector complexes and subsequent IKK activation. Rac1 binding to IL-1R1 appeared to play a central role in mediating Nox2 recruitment into the endosomal compartment following IL-1β stimulation. Rac1 has predominantly been thought to play an essential role in Nox2 activation by recruiting p67phox to the Nox complex (Diekmann et al., 1994). These studies demonstrate for the first time that Rac1 can also serve to localize Nox2 to the proper cellular compartment with a ligand-activated receptor. In contrast to MyD88, Rac1 did not appear to be required for endocytosis of IL-1R1 following ligand binding. However, both effectors contributed to Nox activation in the endosomal compartment, and hence the redox-dependent recruitment of TRAF6 to IL-1R1. In summary, inhibition of MyD88 reduced Nox2 activation and TRAF6 recruitment in the endosomal compartment by inhibiting endocytosis of ligand-activated IL-1R1 (in a similar fashion to dynaminK44A). In contrast, Rac1 inhibition likely reduced Nox2 activation in the endosomal compartment by preventing Nox2 tethering to ligand-activated IL-1R1. However, it is presently unclear if Rac1 binds directly to the receptor or through a secondary unknown effector (other than MyD88).

Oxidation of thiol groups is recognized as a mechanism to induce redox-dependent changes in protein function (Georgiou, 2002; Kamata et al., 2005). Given the ability of H₂O₂ to directly promote TRAF6 recruitment to ligand-activated IL-1R1 at the plasma membrane (at 4° C.) and essentially bypass the need for endocytic formation of redox-active endosomes, oxidation of thiol groups in TRAF6, or an upstream effector such as IRAK, may lead to a redox-dependent change in protein structure that allows for effector recruitment to the IL-1R1/MyD88 complex. Other scenarios are also possible, such as redox dependent changes in MyD88 and/or IL-1R1 that facilitate efficient docking of IRAK/TRAF6 complexes. Alternatively, IRAK/TRAF6 association with IL-1R1 could also be controlled indirectly through ROS regulation of kinases or phosphatases with a catalytic cysteine(s). In support of this later hypothesis, IRAK phosphorylation by PKC has been shown to be critical for IRAK autophosphorylation and NFκB activation by IL-1β (Mamidipudi et al., 2004).

Nox proteins are known to be a major source of ROS within cells following various environmental stimuli (Lambeth, 2004), however, their function in regulating cellular signaling has only recently been recognized. For example, Nox4 appears to be important in ROS-mediated insulin signaling (Mahader et al., 2004), and Nox1 mediates angiotensin II redox-sensitive signaling pathways (Hanna et al., 2002; Lasseque et al., 2001). Here, for the first time, it was shown Nox2 can regulate IL-1β signaling and the mechanism responsible for this redox-dependent regulation in the context of NFκB activation is described. The present findings also provide new insights into the subcellular context in which Nox activation occurs and selectively influences H₂O₂-dependent receptor activation in the endosomal compartment. It is plausible that the presently studied mechanism defining the influences of endosomal Nox-derived ROS on IL-1R1 activation may also have overlapping characteristics with other redox-dependent receptor signaling pathways. For example, PDGF signaling is controlled by H₂O₂ and receptor associated peroxiredoxin II, which acts to eliminate H₂O₂ as the site of receptors and influence PDGFR phosphatases (Choi et al., 2005). ROS production following PDGF stimulation is also controlled by Rac1 and has been suggested to involve NADPH oxidases (Bal et al., 2000). Hence, although the present studies in mammary epithelial cells have implicated endosomal Nox2 in IL-1β signaling, it is possible that other cell types also utilize this mechanism for other redox-regulated signal transduction pathways in conjunction with Rac1-dependent Nox isoforms.

### EXAMPLE 3

Recent studies using controlled expression of mutant SOD1 in motor neurons and microglia have demonstrated that these two cell types contribute to different phases of ALS disease progression, motor neurons in early phases of disease onset and microglia in later phase disease progression (Boillee et al., 2006). These findings implicate primary defects in microglial function as a consequence of mutant SOD1 expression. Hence, although increased numbers of spinal cord microglia in ALS likely enhance the potential for redox-mediated inflammatory damage, the mechanism by which mutant SOD1 alters microglial function and contributes to this inflammatory process remains unknown.

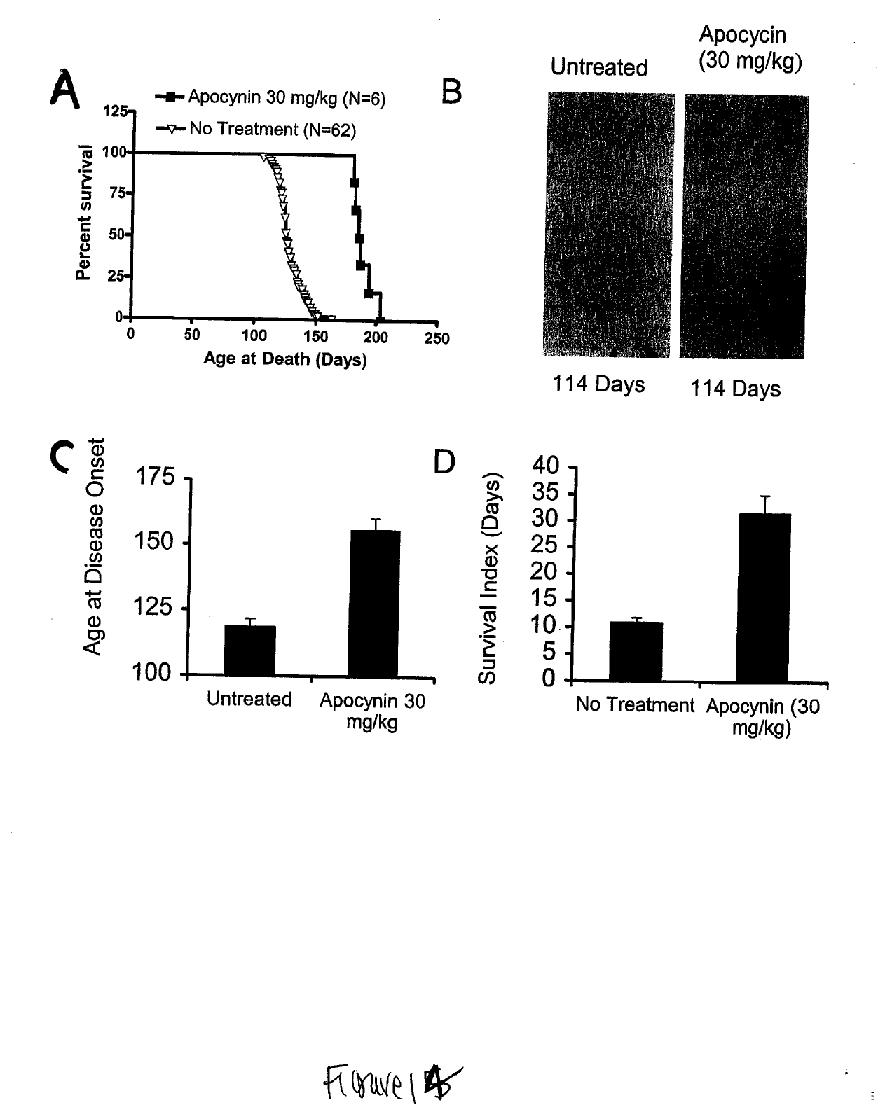

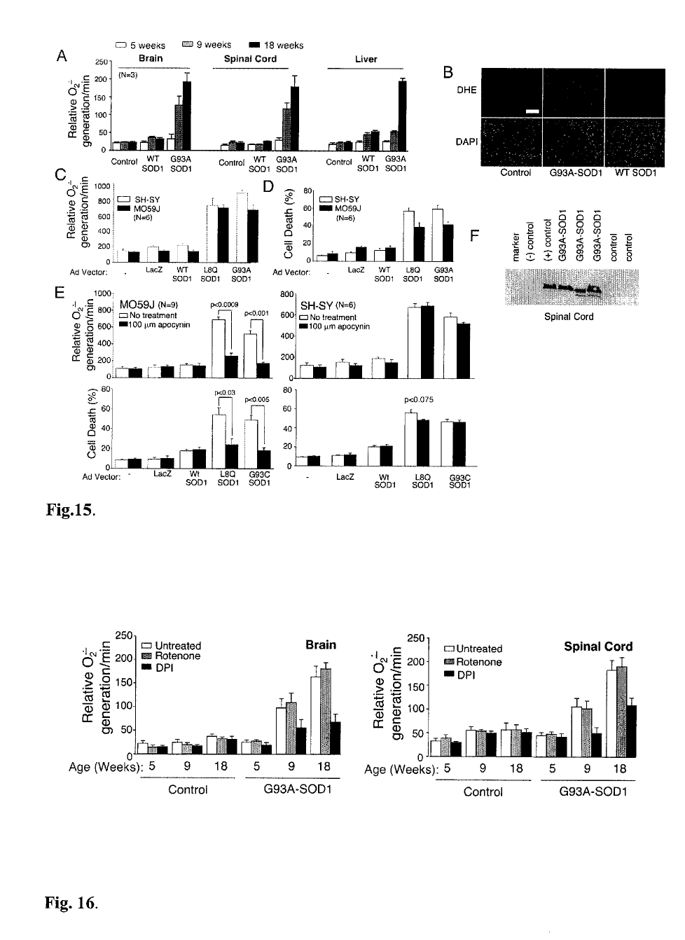

It was hypothesized that mutant SOD1 directly influences the ability of microglia to produce ROS. Given the fact that Nox2^{gp91phox} has been shown to contribute to spinal cord redox-stress in mouse models of ALS (Wu et al., 2006), ALS SOD1 mutants may directly lead to dysregulation of Nox-derived superoxides. Indeed, analysis of transgenic mice overexpressing WT-SOD1 or G93A-SOD1 demonstrated that only mutant forms of SOD1 enhanced NADPH-dependent superoxide production in brain and spinal cord endomembranes (FIGS. 15A-B), which was inhibited by the flavoprotein inhibitor diphenyleneiodionium chloride (DPI), but not mitochondrial complex I inhibitor rotenone (FIG. 16). Interestingly, the liver (FIG. 15A), an organ that does not demonstrate notable pathology in ALS, also demonstrated similar SOD1 mutant-associated increases in Nox activity. In contrast, overexpressing WT-SOD1 in spinal cord and brain did not alter NADPH-dependent superoxide production in endomembranes. Interestingly, WT-SOD1 expression in the liver did significantly increase Nox activity in the liver at 9 and 18 weeks of age, but to a much lesser extent than G93A-SOD1.

To evaluate whether mutant SOD1 proteins could enhance Nox activity directly in the absence of disease-associated inflammatory processes seen in vivo in ALS mice, WT, L8Q, and G93C forms of SOD1 were expressed in both MO59J glial cells and SH-SY neuronal cells using recombinant adenovirus. Overexpression of only the mutant SOD1 proteins enhanced NADPH-dependent [symbol] O₂ production in endomembranes from both glial and neuronal cells type (FIG. 15C) and significantly increased cell death (FIG. 15D). These findings implicate a gain of function in SOD1 mutants that leads to enhanced Nox activation and cellular injury. Apocynin, a known inhibitor of p47phox-regulated NADPH oxidases (Zhang et al., 2006; Furukawa et al., 2004), abrogated SOD1 mutant facilitated NADPH-dependent [symbol] O₂ production only in glial cells with a corresponding increase in cell viability (FIG. 15E). In contrast, apocynin could not inhibit Nox activity in SH-SY neuronal cells and nor did it protect for mutant SOD1-mediated cellular injury.

Apocynin inhibits NADPH oxidases by interfering with recruitment of p47phox to the Nox complex (Stolk et al., 1994). Three known Nox catalytic subunits are regulated by p47phox (Nox1, Nox2, and Nox3)(Ueyama et al., 2005; Lambeth, 2004) and these Nox isoforms are also regulated by the small GTPase Rae (Li et al., 2005). Indeed, both spinal cords of ALS transgenic mice overexpressing the SOD1-G93A mutant demonstrated enhanced Rac1-GTP levels (i.e., activated Rac1) as judged by Pak1 pull-down assays (FIG. 15F). These findings of enhanced Rac1 activation by SOD1 mutant expression led us to the hypothesis that SOD1 might directly interact with Rac1 and/or other Nox complex components to stabilize the activated form of this complex. In support of this hypothesis are findings that SOD1 and Rac1 both recruit to Nox2-active early endosomes following cytokine stimulation (Example 2).

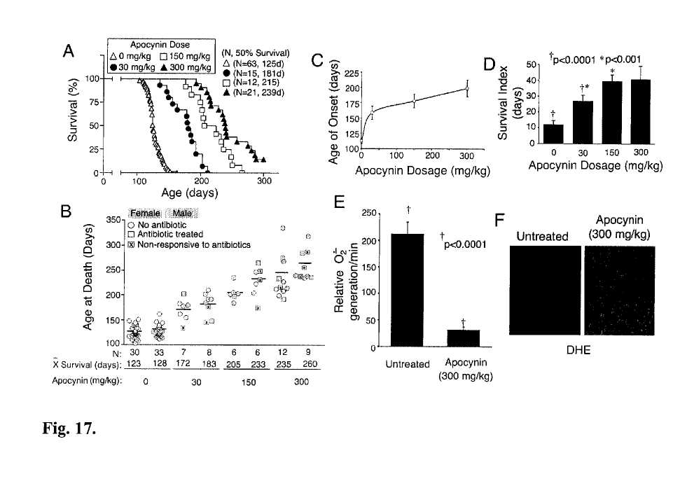

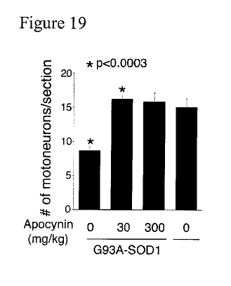

Potential gain of functions in certain ALS associated SOD1 mutations that lead to primary defects in Nox activation sheds new insights into potential pathologic mechanisms in this disease. Recent studies have suggested that deletion of Nox2 prolongs survival in ALS mice (Wu et al., 2006). However, it is currently unclear if other Rac-regulated Nox complexes (such as Nox1 and Nox3) might also contribute to altered ROS production in ALS. To test the therapeutic potential of direct Nox inhibition on the pathoprogression of ALS disease, apocynin in vivo inhibition studies were performed in G93A-ALS mice. Indeed, apocynin administration in the drinking water from 2 weeks of age prolonged survival of G93A-SOD1 mice in a dose-dependent fashion. At the highest dose (300 mg/kg), 50% survival time were increased from 125 days to 239 days (FIG. 17A). This dose also significantly increased the number of motor neurons in the lumbar spinal cord at 120 days (FIG. 19).

There was a clear dose response in the age of onset of disease and survival index (time to death since first signs of symptoms) as judged by 5% weight loss (FIGS. 17C,-D) and gait (data not shown). To confirm that apocynin treatment significantly inhibited NADPH oxidase activity in vivo, terminal stage ALS mice were treated for five days with apocynin in the water and evaluate Nox activity in the spinal cord by lucigenin and DHE assays. These studies demonstrated that apocynin treatment effectively inhibited Nox-derived superoxide production in vivo (FIGS. 17E-F) at later stages of disease associated with microgliosis and increased Nox2 expression (Wu et al., 2006).

One interesting finding was that ALS mice with prolonged survival developed eye infections that if left untreated, led to rapid death without the normal course of motor abnormalities. Treatment of eye infection with systemic antibiotics led to resolution in approximately 50% of cases (FIG. 17B). Importantly, treatment of ALS mice with antibiotics did not increase survival in the absence of apocynin and non-ALS mice treated with apocynin did not develop eye disease. Hence, the eye disease in the G93A-SOD1 mouse model appears to be a previously unobserved feature associated with this model that develops only later in life. The pathologic features of this eye disease include increased exudate containing Staphylococcus aureus. However, no evidence for inflammation in histologic section was observed making the etiology of death difficult to determine (data not shown).

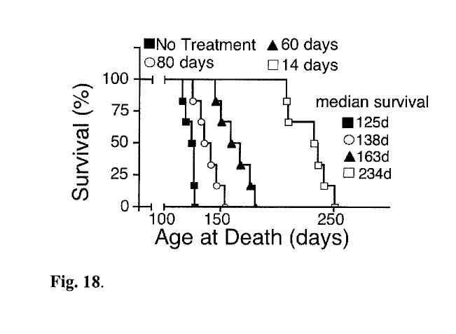

The finding that SOD1 functions to regulate Rac1-dependent superoxide production by NADPH oxidases in a redox-dependent fashion has important implications for ALS and the development of targeted anti-oxidant therapies such as apocynin. The ability of pM quantities of H₂O₂ to liberate SOD1 from Rac-GTP and allow for GTP hydrolysis to occur, suggests that the mechanism of in vivo regulation of Nox may be exquisitely sensitive to small changes in cellular ROS. This mechanism may allow Rac1 to sense and regulate changes in cellular [symbol] O₂ through SOD1 enzymatic conversion to H₂O₂. Such spatial regulation may be a key aspect of SOD1 function as a redox-sensor and the therapeutic effects of apocynin to directly inhibit dysregulated Nox complexes. Furthermore, studies initiating apocynin treatment of ALS mice at 5, 8, and 12 weeks of age demonstrate that inhibition of Nox during early phases of disease is important to the therapeutic effect of apocynin (FIG. 18). Such early phases of disease appear to be most significantly influenced by motor neuron expression of mutant SOD1 (Boilee et al., 2006). Since inhibition of Nox2 expression (Wu et al., 2006) and mutant SOD1 expression (Boilee et al., 2006) in glial cells appears to influence later states of disease associated with inflammatory microgliosis, other Rac1-regulated Nox proteins may be key to development of early states of redox stress in motor neurons that initiate the later phases of inflammatory microgliosis leading to further redox-stress.

### REFERENCES

Abid et al., FEBS Lett., 486:252 (2000).
Abo et al., Nature, 353:668 (1991).
Akira et al., J. Infect. Dis., 187:S356 (2003).
Albrich et al., FEBS Lett., 144:157 (1982).
Aniansson et al., Acta Pathol. Microbiol. Immunol. Scand. Sect. C, 92:357 (1984).
Antunes et al., FEBS Lett., 475:121 (2000).
Babior et al., J. Clin. Invest., 58:989 (1976).
Babior et al., J. Lab. Clin. Med., 85:235 (1975).
Babior, N. Engl. J. Med., 298:659 (1978).
Bae et al., J. Biol. Chem., 272:217 (1997).
Bae et al., J. Biol. Chem., 275:10527 (2000).
Bereznai et al., Neuromuscul. Disord., 7:113 (1997).
Billington et al., Anal. Biochem., 258:251 (1998).
Boillee et al., Science, 312:1389 (2006).
Borregaard et al., Blood, 89:3503 (1997).
Burns et al., Nat. Cell Biol., 2:346 (2000).
Burritt et al., J. Biol. Chem., 270:16974 (1995).
Choi et al., Nature, 435:347 (2005).
Chou and Talalay, Adv. Enzyme Regul., 22:27 (1984).
Clark et al., J. Biol. Chem., 262:4065 (1987).
Clark et al., J. Clin. Invest., 85:714 (1990).
Clark, J. Infect. Dis., 161:1140 (1990).
Cleveland, Neuron, 24:515 (1999).
Coligan, Current protocols in immunology. Greene Pub. Associates and Wiley-Interscience, New York (1991).
Conner et al., Nature, 422:37 (2003).
Dahlgren et al., Biolumin. Chemilumin., 4:263 (1989).
Dahlgren et al., Infect. Immun., 47:326 (1985).
DeLeo et al., J. Leukoc. Biol., 60:677 (1996).
DeLeo et al., Proc. Natl. Acad. Sci. USA, 92:7110 (1995).
Deshpande et al., Faseb. J., 14:1705 (2000).
Diekmann et al., Science, 265:531 (1994).
Dinauer et al., Nature, 327:717 (1987).
Dinauer, et al., Crit. Rev. Clin. Lab. Sci., 30:329 (1993).
Dorseuil et al., J. Leukocyte Biol., 58:108 (1995).
Duan et al., J. Virol., 73:10371 (1999).
Engelhardt, Antioxid. Redox. Signal, 1:5 (1999).
Enyedi et al., Cell, 70:879 (1992).
Fantone, et al., Hum. Pathol., 16:973 (1985).
Faulkner et al., Free Radic. Biol. Med., 15:447 (1993).
Fearon et al., Science, 272:50 (1996).
Finan et al., J. Biol. Chem., 269:13752 (1994).
Freeman et al., J. Immunol. Methods, 139:241 (1991).
Frey et al., Circ. Res., 90:1012 (2002).
Furukawa et al., J. Clin. Invest., 114:1752 (2004).
Gabig et al., Blood, 85:804 (1995).
Georgiou, Cell, 111:607 (2002).
Ghezzo-Schoneich et al., Free Radic. Biol. Med., 30:858 (2001).
Ghosh et al., Cell, 109:S81 (2002).
Graham et al., Anal. Biochem., 220:367 (1994).
Graham et al., Z. Gastroenterol., 34:76 (1996).
Graham, Scientific World Journal, 2:1400 (2002).
Gu et al., J. Biol. Chem., 278:17210 (2003).
Gurney et al., Science, 264:1772 (1994).
Halliwell et al., Free radicals in biology and medicine, Third Edition; Oxford Science Publications, Oxford, UK., p. 388 (1998).
Halliwell, B. and Gutteridge, J. M. C. Free radicals in biology and medicine, Third Edition; Oxford Science Publications, pp 33-34 (1998).
Halliwell, Cell. Biol. Int. Rep., 2:113 (1978).
Hampton, Blood, 92:3007 (1998).
Hanna et al., Antioxid. Redox. Signal, 4:899 (2002).
Harrison et al., J. Biol. Chem., 251:1371 (1976).
Hayakawa et al., Embo J., 22:3356 (2003).
Heyworth et al., J. Biol. Chem., 269:30749 (1994).
Heyworth et al., J. Clin. Invest., 87:352 (1991).
Hirshberg et al., Nat. Struct. Biol., 4:147 (1997).
Hoffmann et al., Science, 284:1313 (1999).
Hordijk, Circ. Res., 98:453 (2006).
Huang et al., Proc. Natl. Acad. Sci. USA, 94:12829 (1997).
Huffman et al., J. Org. Chem., 60:1590 (1995).
Hyslop et al., Free Radic. Biol. Med., 19:31 (1995).
Irani et al., Science, 275:1649 (1997).
Ito et al., Biochemistry, 36:9109 (1997).
Iyer et al., J. Biol. Chem., 269:22405 (1994).
Jackson et al., Hematol. Oncol. Clin. North. Am., 2:317 (1988).
Janeway et al., Cell, 76:275 (1994).
Jefferies et al., Mol. Cell. Biol., 21:4544 (2001).
Kamata et al., Cell, 120:649 (2005).
Kanai et al., Nat. Cell Biol., 3:675 (2001).
Kang et al., J. Biol. Chem., 279:2535 (2004).
Kettle et al., Redox Rep., 3:3 (1997).
Kheradmand et al., Science, 280:898 (1998).
Kim et al., J. Neurol. Sci., 206:65 (2003).
Klebanoff, J. Bacteriol., 95:2131 (1968).
Klebanoff, Proc. Assoc. Am. Physicians, 111:383 (1999).
Kleinberg et al., J. Biol. Chem., 265:15577 (1990).
Kretz-Remy et al., J. Cell Biol., 133:1083 (1996).
Kwon et al., J. Biol. Chem., 275:423 (2000).
Lambeth, Nat. Rev. Immunol., 4:181 (2004).
Laperre et al., FEBS Lett., 443:235 (1999).
Lassegue et al., Circ. Res., 88:888 (2001).
Leto et al., Proc. Natl. Acad. Sci. U.S.A., 91:10650 (1994).
Leusen et al., J. Clin. Invest., 93:2120 (1994).
Leusen et al., J. Exp. Med., 180:2329 (1994).
Li et al., Antioxid. Redox. Signal, 3:415 (2001).
Li et al., Circ. Res., 90:143 (2002).
Li et al., J. Biol. Chem., 273:2015 (1998).
Li et al., Proc. Natl. Acad. Sci. USA, 99:5567 (2002).
Liochev et al., Arch. Biochem. Biophys., 337:115 (1997).
Lomax et al., Science, 245:409 (1989)
Lomax et al., Science, 246:987 (1989).
Mahadev et al., Mol. Cell. Biol., 24:1844 (2004).
Mamidipudi et al., J. Biol. Chem., 279:4161 (2004).
Manser et al., Mol. Cell, 1:183 (1998).
Matzuk et al., Endocrinology, 139:4008 (1998).
McCord et al., J. Biol. Chem., 244:6049 (1969).
McNally et al., J. Biolumin. Chemilumin., 11:99 (1996).
Medicinal Plants of Nepal, p. 37, H. M. G. Press, Kathmandu (1970).
Menard et al., Eur. J. Biochem., 206:537 (1992).
Muijsers et al., Br. J. Pharmacol., 130:932 (2000).
Muzio et al., Science, 278:1612 (1997).
Nakanishi et al., J. Biol. Chem., 267:19072 (1992).
Nauseef et al., Mandell, G. L., Bennett, J. E. and Dolin, R. (Eds.); Fifth Edition, Churchill Livingstone, Philadelphia, USA., Chapter 8: Granulocytic Phagocytes (2000).
Nauseef, Hematol. Oncol. Clin. North Am., 2:135 (1988).
Needleman and Wunsch, J. Mol. Biol., 48:443 (1970).
Ogle et al., J. Immunol. Methods, 115:17 (1988).
O'Neill et al., J. Leukoc. Biol., 63:650 (1998).
Pagano et al., Proc. Natl. Acad. Sci. USA, 94:14483 (1997).
Pandey et al., Abst. 15th Annual Conference, Indian Pharmacological Society.
Pandey et al., J. Res. Ind. Med., 5:11 (1970).
Park et al., J. Biol. Chem., 267:17327 (1992).
Park et al., Mol. Cell. Biol., 24:4384 (2004).
Pearson and Lipman, Proc. Natl. Acad. Sci. USA, 85:2444 (1988).
Plonne et al., Anal. Biochem., 276:88 (1999).
Pollock et al., Nat. Genet., 9:202 (1995).
Puceat et al., Mol. Biol. Cell, 14:2781 (2003).
Qian et al., J. Biol. Chem., 276:41661 (2001).
Quinn et al., J. Biol. Chem., 268:20983 (1993).
Quinn et al., Nature, 342:198 (1989).
Rac et al., Science, 284:805 (1999).
Regnier et al., Cell, 90:373 (1997).
Remington's Pharmaceutical Sciences, 17th ed., Mack Publishing Company, Easton, Pa., p. 1418 (1985),
Rhee et al., J. Am. Soc. Nephrol., 14:S211 (2003).
Rhee et al., Sci. STKE, 53:1 (2000).
Rittinger et al., Nature, 388:693 (1997).
Rittinger et al., Nature, 389:758 (1997).
Roos et al., J. Biol. Chem., 259:1770 (1984).
Rosen et al., J. Exp. Med., 149:27 (1979).
Rothwarf et al., Sci. STKE, 1999:RE1 (1999).
Rotrosen et al., Science, 256:1459 (1992).
Samuni et al., J. Biol. Chem. 263:13797 (1988).
Sanlioglu et al., J. Biol. Chem., 276:30188 (2001).
Schettler et al., Eur. J. Biochem., 197:197 (1991).
Schnitzler et al., Adv. Exp. Med. Biol., 418:897 (1997).
Segal et al., Biochem. J., 284:781 (1992).
Segal et al., Trends Biochem. Sci., 18:43 (1993).
Segal, Nature, 326:88 (1987).
Smith et al., Blood, 77:673 (1991).
Smith and Waterman, Adv. Appl. Math., 2:482 (1981).
Sorkin et al., Nat. Rev. Mol. Cell. Biol., 3:600 (2002).
Sprang, Annu. Rev. Biochem., 66:639 (1997).
Stolk et al., Am. J. Respir. Cell. Mol. Biol., 11:95 (1994).
Sulciner et al., Mol. Cell. Biol., 16:7115 (1996).
Sumimoto et al., Proc. Natl. Acad. Sci. USA, 91:5345 (1994).
Sundaresan et al., Science, 270:296 (1995).
Supinski et al., J. Appl. Physiol., 87:776 (1999).
Topp et al., J. Biol. Chem., 279:24612 (2004).
Trischler et al., J. Cell Sci., 112:4773 (1999).
Tucker et al., J. Med. Chem., 37:2437 (1994).
Ueyama et al., Mol. Cell. Biol., 26:2160 (2006).
Vaidya et al., Ass. Phys. Ind. Conf. Abstracts (1981).
Van Buul et al., Antioxid. Redox. Signal, 7:308 (2005).
Van Dalen et al., Biochem. J., 327:487 (1997).
Vliet et al., J. Biol. Chem., 272:7617 (1997).
Volpp et al., Science, 242:1295 (1988).
Wang et al., Nature, 412:346 (2001).
Weinbaum et al., Nature, 286:725 (1980).
Weiss, N. Engl. J. Med. 320:365 (1989).
Wesche et al., Immunity, 7:837 (1997).
Whitin et al., Blood, 66:1182 (1985).
Wientjes et al., Biochem. J., 296:557 (1993).
Williams et al., Proc. Natl. Acad. Sci. USA, 74:1204 (1977).
Worthylake et al., Nature, 408:682 (2000).
Wu et al., Proc. Natl. Acad. Sci. USA, 103:12132 (2006).
Xia et al., Biochemistry, 37:16465 (1998).
Xiao et al., Am. J. Physiol. Cell Physiol., 282:C926 (2002).
Yamaoka-Tojo et al., Circ. Res., 95:276 (2004).
Yang et al., Nat. Genet., 29:160 (2001).
Zerial et al., Nat. Rev. Mol. Cell. Biol., 2:107 (2001).
Zipfel et al., Biochem. Biophys. Res. Commun., 232:209 (1997).
Zwacka et al., Nat. Med., 4:698 (1998).
Zhang et al., J. Clin. Invest., 116:3050 (2006).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed:

1. A method to detect or determine one or more agents that inhibit the binding of a GTPase to SOD, comprising:
a) contacting one or more agents, isolated GTPase and SOD protein under conditions that allow for binding of GTPase to SOD; and
b) detecting or determining whether the one or more agents inhibit binding of the isolated GTPase to the SOD protein.

2. The method of claim 1 wherein the isolated GTPase or the SOD protein is immobilized.

3. The method of claim 1 wherein the isolated GTPase is recombinant GTPase.

4. The method of claim 3 wherein the isolated GTPase is Rac or RhoA.

5. The method of claim 4 wherein the isolated Rac protein comprises TVFDNYSANVMVDGKPVNLGLWDTAGGEDYDRLRPL (SEQ ID NO:2).

6. The method of claim 1 wherein the SOD protein is recombinant SOD protein.

7. The method of claim 1 wherein the SOD protein is isolated SOD protein.

8. The method of claim 7 wherein the isolated SOD protein is a fusion protein.

9. The method of claim 7 wherein the isolated SOD protein is labeled.

10. The method of claim 1 wherein the SOD protein has a substitution relative to wild-type SOD that enhances binding to the GTPase.

11. The method of claim 1 wherein an antibody is employed to identify whether one or more agents inhibit binding.

12. The method of claim 1 wherein the isolated GTPase is a fusion protein.

13. The method of claim 1 wherein the isolated GTPase is labeled.

14. The method of claim 9 or 13 wherein the label is a fluorophore.

15. The method of claim 1 wherein the one or more agents are contacted with the isolated GTPase before the SOD protein.

16. The method of claim 1 wherein a library of agents is contacted with the isolated GTPase and the SOD protein.

17. A method to identify one or more agents that inhibit the binding of GTPase to SOD, comprising:
a) providing a mixture comprising one or more agents and a sample comprising GTPase and SOD protein;
b) subjecting the mixture to conditions that allow for binding of the GTPase to the SOD protein; and
c) identifying whether the one or more agents inhibit the binding of the GTPase to the SOD protein.

18. The method of claim 17 wherein the sample comprises lysed cells.

19. The method of claim 17 wherein the sample comprises a membrane fraction.

20. The method of claim 17 wherein the sample comprises isolated endosomes or endosome membranes comprising Nox.

21. The method of claim 20 wherein the Nox is Nox1 or Nox2.

22. The method of claim 17 wherein the sample comprises intact cells.

23. The method of claim 17 wherein the GTPase is a fusion protein.

24. The method of claim 17 wherein the GTPase is labeled.

25. The method of claim 17 wherein the GTPase is Rac or RhoA.

26. The method of claim 17 wherein the SOD protein is a fusion protein.

27. The method of claim 17 wherein the SOD protein is labeled.

28. The method of claim 4 or 25 wherein the Rac protein is Rac1 or Rac2.

29. One or more agents identified by the method of claim 1 or 17.

30. An isolated peptide which binds SOD, wherein the peptide has at least 90% identity to SEQ ID NO:2, and wherein the peptide is not SEQ ID NO: 1 (full-length Rac1), residues 1 to 177 of SEQ ID NO:1, SEQ ID NO:3 (full-length Rac2), or SEQ ID NO:4 (full-length RhoA).

31. The isolated peptide of claim 30 which comprises SEQ ID NO:2.

32. The isolated peptide of claim 30 which includes residues corresponding to residues 1 to 88 of SEQ ID NO:1.

33. The isolated peptide of claim 30 which includes residues corresponding to residues 1 to 116 of SEQ ID NO:1.

34. The isolated peptide of claim 30 which is fused to a heterologous peptide to yield a fusion protein.

35. The isolated peptide of claim 30 or 34 which is immobilized.

36. A kit comprising a fusion protein comprising a GTPase which comprises a SOD binding region, a fusion protein comprising SOD which comprises a GTPase binding region, or a combination thereof.

37. The kit of claim 36 wherein the GTPase or the SOD is fused to a peptide or protein suitable to immobilize the fusion protein.

38. The kit of claim 36 wherein the GTPase or the SOD is fused to a fluorescent protein.

39. The kit of claim 36 wherein the fusion protein is the fusion protein of claim 34.

40. The kit of claim 36 wherein the GTPase is Rho or Rac.

41. A method to inhibit or treat a neuronal degenerative disease in a mammal, comprising administering to a mammal in need thereof a composition comprising an effective amount of an inhibitor of NADPH oxidase.

42. The method of claim 41 wherein the inhibitor is a compound of formula (I).

43. The method of claim 41 wherein the disease is ALS.

44. The method of claim 41 wherein the composition comprises a plant extract.

45. The method of claim 44 wherein the plant is a Picrorhiza plant.

46. The method of claim 41 wherein the inhibitor inhibits the binding of SOD to a GTPase.

47. The method of claim 41 wherein the mammal is a human.

48. The method of claim 41 wherein the inhibitor inhibits p47phox.

49. The method of claim 41 wherein the inhibitor is orally administered.

50. The method of claim 41 wherein the administration is daily.


SEQUENCE LISTING (27 sequences)

SEQ ID NO: 1 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 2 - protein, 36 aa, Homo sapiens
TVFDNYSANVMVDGKPVNLGLWDTAGGEDYDRLRPL

SEQ ID NO: 3 - protein, 192 aa, Homo sapiens
MQAIKCVVVGDGAVGKTCLLISYTTNAFPGEYIPTVFDNYSANVMVDSKPVNLGLWDTAGQEDYDRLRPLSYPQTDVFLICFSLVSPASYENVRAKWFPEVRHHCPSTPIILVGTKLDLRDDKDTIEKLKEKKLAPITYPQGLALAKEIDSVKYLECSALTQRGLKTVFDEAIRAVLCPQPTRQQKRACSLL

SEQ ID NO: 4 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 5 - protein, 193 aa, Homo sapiens; identical to SEQ ID NO: 4

SEQ ID NO: 6 - protein, 154 aa, Homo sapiens [sequence omitted]

SEQ ID NO: 7 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 8 - protein, 1657 aa, Homo sapiens
MDSKKRSSTEAEGSKERGLVHIWQAGSFPITPERLPGWGGKTVLQAALGVKHGVLLTEDGEVYSFGTLPWRSGPVEICPSSPILENALVGQYVITVATGSFHSGAVTDNGVAYMWGENSAGQCAVANQQYVPEPNPVSIADSEASPLLAVRILQLACGEEHTLALSISREIWAWGTGCQLGLITTAFPVTKPQKVEHLAGRVVLQVACGAFHSLALVQCLPSQDLKPVPERCNQCSQLLITMTDKEDHVIISDSHCCPLGVTLTESQAENHASTALSPSTETLDRQEEVFENTLVANDQSVATELNAVSAQITSSDAMSSQQNVMGTTEISSARNIPSYPDTQAVNEYLRKLSDHSVREDSEHGEKPVPSQPLLEEAIPNLHSPPTTSTSALNSLVVSCASAVGVRVAATYEAGALSLKKVMNFYSTTPCETGAQAGSSAIGPEGLKDSREEQVKQESMQGKKSSSLVDIREEETEGGSRRLSLPGLLSQVSPRLLRKAARVKTRTVVLTPTYSGEADALLPSLRTEVWTWGKGKEGQLGHGDVLPRLQPLCVKCLDGKEVIHLEAGGYHSLALTAKSQVYSWGSNTFGQLGHSDFPTTVPRLAKISSENGVWSIAAGRDYSLFLVDTEDFQPGLYYSGRQDPTEGDNLPENHSGSKTPVLLSCSKLGYISRVTAGKDSYLALVDKNIMGYIASLHELATTERRFYSKLSDIKSQILRPLLSLENLGTTTTVQLLQEVASRFSKLCYLIGQHGASLSSFLHGVKEARSLVILKHSSLFLDSYTEYCTSITNFLVMGGFQLLAKPAIDFLNKNQELLQDLSEVNDENTQLMEILNTLFFLPIRRLHNYAKVLLKLATCFEVASPEYQKLQDSSSCYECLALHLGRKRKEAEYTLGFWKTFPGKMTDSLRKPERRLLCESSNRALSLQHAGRFSVNWFILFNDALVHAQFSTHHVFPLATLWAEPLSEEAGGVNGLKITTPEEQFTLISSTPQEKTKWLRAISQAVDQALRGMSDLPPYGSGSSVQRQEPPISRSAKYTFYKDPRLKDATYDGRWLSGKPHGRGVLKWPDGKMYSGMFRNGLEDGYGEYRIPNKAMNKEDHYVGHWKEGKMCGQGVYSYASGEVFEGCFQDNMRHGHGLLRSGKLTSSSPSMFIGQWVMDKKAGYGVFDDITRGEKYMGMWQDDVCQGNGVVVTQFGLYYEGNFHLNKMMGNGVLLSEDDTIYEGEFSDDWTLSGKGTLTMPNGDYIEGYFSGEWGSGIKITGTYFKPSLYESDKDRPKVFRKLGNLAVPADEKWKAVFDECWRQLGCEGPGQGEVWKAWDNIAVALTTSRRQHRDSPEILSRSQTQTLESLEFIPQHVGAFSVEKYDDIRKYLIKACDTPLHPLGRLVETLVAVYRMTYVGVGANRRLLQEAVKEIKSYLKRIFQLVRFLFPELPEEGSTIPLSAPLPTERKSFCTGKSDSRSESPEPGYVVTSSGLLLPVLLPRLYPPLFMLYALDNDREEDIYWECVLRLNKQPDIALLGFLGVQRKFWPATLSILGESKKVLPTTKDACFASAVECLQQISTTFTPSDKLKVIQQTFEEISQSVLASLHEDFLWSMDDLFPVFLYVVLRARIRNLGSEVHLIEDLMDPYLQHGEQGIMFTTLKACYYQIQREKLN

SEQ ID NO: 9 - protein, 4 aa, Homo sapiens
LKGD

SEQ ID NO: 10 - protein, 8 aa, Artificial Sequence
  Consensus sequence
  SITE 4: Xaa = Thr or Arg
  SITE 5: Xaa = Gln or Ala
GDNXXGCT

SEQ ID NO: 11 - protein, 152 aa, Bos taurus
MATKAVCVLKGDGPVQGTIHFEAKGDTVVVTGSITGLTEGDHGFHVHQFGDNTQGCTSAGPHFNPLSKKHGGPKDEERHVGDLGNVTADKNGVAIVDIVDPLISLSGEYSIIGRTMVVHEKPDDLGRGGNEESTKTGNAGSRLACGVIGIAK

SEQ ID NO: 12 - protein, 151 aa, Ovis aries
ATKAVCVLKGDGPVQGTIRFEAKGDKVVVTGSITGLTEGDHGFHVHQFGDNTQGCTSAGPHFNPLSKKHGGPKDEERHVGDLGNVKADKNGVAIVDIVDPLISLSGEYSIIGRTMVVHEKPDDLGRGGNEESTKTGNAGGRLACGVIGIAP

SEQ ID NO: 13 - protein, 152 aa, Odocoileus virginianus
MATKAVCVLKGDGPVQGTIRFEAKGHTVVVTGSITGLTEGDHGFHVHQFGDNTQGCTSAGPHFNPLSKKHGGPKDEERHVGDLGNVTADKNGVAKVDIVDSLISLSGEHSIIGRTMVVHEKPDDLGRGGNEESTKTGNARNRLACGVIGIAQ

SEQ ID NO: 14 - protein, 152 aa, Sus domestica
ATKAVCVLKGDGPVQGTIYFELKGEKTVLVTGTIKGLAEGDHGFHVHQFGDNTQGCTSAGPHFNPESKKHGGPKDQERHVGDLGNVTAGKDGVATVYIEDSVIALSGDHSIIGRTMVVHEKPDDLGRGGNEESTKTGNAGSRLACGVIGITQ

SEQ ID NO: 15 - protein, 153 aa, Oryctolagus cuniculus
MATKAVCVLKGDGPVEATIHFEQKGTGPVVVKGRITGLTEGLHEFHVHQFGDNRQGCTSAGPHFNPLSKKHGGPKDEERHVGDLGNVTAGSNGVADVLIEDSVISLSGDMSVIGRTLVVHEKEDDLGKGGNDESTKTGNAGSRLACGVIGISP

SEQ ID NO: 16 - protein, 153 aa, Candida albicans
MATKAVCVLKGDGPVQGIIHFEQKANGPVVVKGRITGLVEGKHGFHVHEFGDNTQGCTSAGPHFNPLSKKHGGPQDEERHVGDLGNVTAGADGVANVSIEDSLISLSGANSIIGRTMVVHEKPDDLGKGGNEESTKTGNAGSRLACGVIGIAQ

SEQ ID NO: 17 - protein, 154 aa, Homo sapiens [sequence omitted]

SEQ ID NO: 18 - protein, 154 aa, Mus musculus
MAMKAVCVLKGDGPVQGTIHFEQKASGEPVVLSGQITGLTEGQHGFHVHQYGDNTQGCTSAGPHFNPHSKKHGGPADEERHVGDLGNVTAGKDGVANVSIEDRVISLSGEHSIIGRTMVVHEKQDDLGKGGNEESTKTGNAGSRLACGVIGIAQ

SEQ ID NO: 19 - protein, 154 aa, Rattus norvegicus
MAMKAVCVLKGDGPVQGVIHFEQKASGEPVVVSGQITGLTEGEHGFHVHQYGDNTQGCTTAGPHFNPHSKKHGGPADEERHVGDLGNVAAGKDGVANVSIEDRVISLSGEHSIIGRTMVVHEKQDDLGKGGNEESTKTGNAGSRLACGVIGIAQ

SEQ ID NO: 20 - protein, 154 aa, Equus caballus
MALKAVCVLKGDGPVHGVIHFEQQQEGGPVVLKGFIEGLTKGDHGFHVHEFGDNTQGCTTAGAHFNPLSKKHGGPKDEERHVGDLGNVTADENGKADVDMKDSVISLSGKHSIIGRTMVVHEKQDDLGKGGNEESTKTGNAGSRLACGVIGIAP

SEQ ID NO: 21 - protein, 36 aa, Homo sapiens
TVFDNYSANVMVDGKPVNLGLWDTAGQEDYDRLRPL

SEQ ID NO: 22 - protein, 36 aa, Homo sapiens
TVFDNYSANVMVDSKPVNLGLWDTAGQEDYDRLRPL

SEQ ID NO: 23 - protein, 36 aa, Homo sapiens
TVFENYVADIEVDGKQVELALWDTAGQEDYDRLRPL

SEQ ID NO: 24 - protein, 36 aa, Homo sapiens
TVFDNYAVTVMIGGEPYTLGLFDTAGQEDYDRLRPL

SEQ ID NO: 25 - protein, 36 aa, Artificial Sequence
  A synthetic peptide
  SITE 4: Xaa = Asp or Glu
  SITE 7: Xaa = Ser or Val
  SITE 9: Xaa = Asn or Asp
  SITE 10: Xaa = Val or Ile
  SITE 11: Xaa = Met or Glu
  SITE (14)...(14): Xaa = Gly or Ser
  SITE (16)...(16): Xaa = Pro or Gln
  SITE (18)...(18): Xaa = Asn or Glu
  SITE (20)...(20): Xaa = Gly or Ala
TVFXNYXAXXXVDXKXVXLXLWDTAGQEDYDRLRPL

SEQ ID NO: 26 - DNA, 33 bp, Artificial Sequence
  A synthetic oligonucleotide
aaggccgtgtgcgtgcagaagggcgacggccca

SEQ ID NO: 27 - DNA, 33 bp, Artificial Sequence
  A synthetic oligonucleotide
aaggccgtgtgcgtgctgaaggttgacggccca